SEQUENCE LISTING (24 sequences)

SEQ ID NO: 1 - DNA, 3147 bp, Homo Sapien
  CDS (23)...(2589): Nucleotide sequence encoding MTSP1
  ref <301>: AUTHORS: O′Brien, T.J. and Tanimoto, H.
  ref <308>: DATABASE ACCESSION NUMBER: GenBank #AR081724
  ref <309>: DATABASE ENTRY DATE: 2000-08-31
  ref <310>: PATENT DOCUMENT NUMBER: 5,972,616
  ref <311>: PATENT FILING DATE: 1998-02-20
  ref <312>: PUBLICATION DATE: 1999-10-26
tcaagagcggcctcggggtaccatggggagcgatcgggcccgcaagggcggagggggcccgaaggacttcggcgcgggactcaagtacaactcccggcacgagaaagtgaatggcttggaggaaggcgtggagttcctgccagtcaacaacgtcaagaaggtggaaaagcatggcccggggcgctgggtggtgctggcagccgtgctgatcggcctcctcttggtcttgctggggatcggcttcctggtgtggcatttgcagtaccgggacgtgcgtgtccagaaggtcttcaatggctacatgaggatcacaaatgagaattttgtggatgcctacgagaactccaactccactgagtttgtaagcctggccagcaaggtgaaggacgcgctgaagctgctgtacagcggagtcccattcctgggcccctaccacaaggagtcggctgtgacggccttcagcgagggcagcgtcatcgcctactactggtctgagttcagcatcccgcagcacctggtggaggaggccgagcgcgtcatggccgaggagcgcgtagtcatgctgcccccgcgggcgcgctccctgaagtcctttgtggtcacctcagtggtggctttccccacggactccaaaacagtacagaggacccaggacaacagctgcagctttggcctgcacgcccgcggtgtggagctgatgcgcttcaccacgcccggcttccctgacagcccctaccccgctcatgcccgctgccagtgggccctgcggggggacgccgactcagtgctgagcctcaccttccgcagctttgaccttgcgtcctgcgacgagcgcggcagcgacctggtgacggtgtacaacaccctgagccccatggagccccacgccctggtgcagttgtgtggcacctaccctccctcctacaacctgaccttccactcctcccagaacgtcctgctcatcacactgataaccaacactgagcggcggcatcccggctttgaggccaccttcttccagctgcctaggatgagcagctgtggaggccgcttacgtaaagcccaggggacattcaacagcccctactacccaggccactacccacccaacattgactgcacatggaacattgaggtgcccaacaaccagcatgtgaaggtgagcttcaaattcttctacctgctggagcccggcgtgcctgcgggcacctgccccaaggactacgtggagatcaatggggagaaatactgcggagagaggtcccagttcgtcgtcaccagcaacagcaacaagatcacagttcgcttccactcagatcagtcctacaccgacaccggcttcttagctgaatacctctcctacgactccagtgacccatgcccggggcagttcacgtgccgcacggggcggtgtatccggaaggagctgcgctgtgatggctgggccgactgcaccgaccacagcgatgagctcaactgcagttgcgacgccggccaccagttcacgtgcaagaacaagttctgcaagcccctcttctgggtctgcgacagtgtgaacgactgcggagacaacagcgacgagcaggggtgcagttgtccggcccagaccttcaggtgttccaatgggaagtgcctctcgaaaagccagcagtgcaatgggaaggacgactgtggggacgggtccgacgaggcctcctgccccaaggtgaacgtcgtcacttgtaccaaacacacctaccgctgcctcaatgggctctgcttgagcaagggcaaccctgagtgtgacgggaaggaggactgtagcgacggctcagatgagaaggactgcgactgtgggctgcggtcattcacgagacaggctcgtgttgttgggggcacggatgcggatgagggcgagtggccctggcaggtaagcctgcatgctctgggccagggccacatctgcggtgcttccctcatctctcccaactggctggtctctgccgcacactgctacatcgatgacagaggattcaggtactcagaccccacgcagtggacggccttcctgggcttgcacgaccagagccagcgcagcgcccctggggtgcaggagcgcaggctcaagcgcatcatctcccaccccttcttcaatgacttcaccttcgactatgacatcgcgctgctggagctggagaaaccggcagagtacagctccatggtgcggcccatctgcctgccggacgcctcccatgtcttccctgccggcaaggccatctgggtcacgggctggggacacacccagtatggaggcactggcgcgctgatcctgcaaaagggtgagatccgcgtcatcaaccagaccacctgcgagaacctcctgccgcagcagatcacgccgcgcatgatgtgcgtgggcttcctcagcggcggcgtggactcctgccagggtgattccgggggacccctgtccagcgtggaggcggatgggcggatcttccaggccggtgtggtgagctggggagacggctgcgctcagaggaacaagccaggcgtgtacacaaggctccctctgtttcgggactggatcaaagagaacactggggtataggggccggggccacccaaatgtgtacacctgcggggccacccatcgtccaccccagtgtgcacgcctgcaggctggagactggaccgctgactgcaccagcgcccccagaacatacactgtgaactcaatctccagggctccaaatctgcctagaaaacctctcgcttcctcagcctccaaagtggagctgggaggtagaaggggaggacactggtggttctactgacccaactgggggcaaaggtttgaagacacagcctcccccgccagccccaagctgggccgaggcgcgtttgtgtatatctgcctcccctgtctgtaaggagcagcgggaacggagcttcggagcctcctcagtgaaggtggtggggctgccggatctgggctgtggggcccttgggccacgctcttgaggaagcccaggctcggaggaccctggaaaacagacgggtctgagactgaaattgttttaccagctcccagggtggacttcagtgtgtgtatttgtgtaaatgggtaaaacaatttatttctttttaaaaaaaaaaaaaaaaaaa

SEQ ID NO: 2 - protein, 855 aa, Homo Sapien [sequence omitted]

SEQ ID NO: 3 - DNA, 3147 bp, Homo Sapien; identical to SEQ ID NO: 1
  CDS (1865)...(2590): Nucleic acid sequence of protease domain of MTSP1

SEQ ID NO: 4 - protein, 241 aa, Homo Sapien [sequence omitted]

SEQ ID NO: 5 - DNA, 804 bp, Homo Sapien
  CDS (1)...(804): Nucleotide sequence encoding MTSP20, including MTSP20 protease domain
acagcaggtccccaggcaggagcaccctccccatggccctgggaggccaggctgatgcaccagggacagctggcctgtggcggagccctggtgtcagaggagacggtgctaactgttgcccactgcttcattgggcgccaggccccagaggaatggagcgtagggctggggaccagaccggaggagtggggcctgaagcagctcatcctgcatggagcctacacccaccctgaggggggctacgacatggccctcctgctgctggcccagcctgtgacactgggagccagcctgcggcccctctgcctgccctatcctgaccaccacctgcctgatggggagcgtggctgggttctgggacgggcccgcccaggagcaggcatcagctccctccagacagtgcccgtgaccctcctggggcctagggcctgcagccggctgcatgcagctcctgggggtgatggcagccctattctgccggggatggtgtgtaccagtgctgtgggtgagctgcccagctgtgagggcctgtctggggcaccactggtgcatgaggtgaggggcacatggttcctggccgggctgcacagcttcggagatgcttgccaaggccccgccaggccggcggtcttcaccgcgctccctgcctatgaggactgggtcagcagtttggactggcaggtctacttcgccgaggaaccagagcccgaggctgagcctggaagctgcctggccaacatgagtatgtggccccggggcctcctgccaaaccctgcctctccaggacccttctctctccagtga

SEQ ID NO: 6 - protein, 267 aa, Homo Sapien
TAGPQAGAPSPWPWEARLMHQGQLACGGALVSEETVLTVAHCFIGRQAPEEWSVGLGTRPEEWGLKQLILHGAYTHPEGGYDMALLLLAQPVTLGASLRPLCLPYPDHHLPDGERGWVLGRARPGAGISSLQTVPVTLLGPRACSRLHAAPGGDGSPILPGMVCTSAVGELPSCEGLSGAPLVHEVRGTWFLAGLHSFGDACQGPARPAVFTALPAYEDWVSSLDWQVYFAEEPEPEAEPGSCLANMSMWPRGLLPNPASPGPFSLQ

SEQ ID NO: 7 - DNA, 30 bp, Artificial Sequence
  5′ end primer
gaagcagctcatcctgcatggagcctacac

SEQ ID NO: 8 - DNA, 27 bp, Artificial Sequence
  3′ end primer
ggccaggaaccatgtgcccctcacctc

SEQ ID NO: 9 - DNA, 30 bp, Artificial Sequence; identical to SEQ ID NO: 7
  PCR 5′ end primer

SEQ ID NO: 10 - DNA, 27 bp, Artificial Sequence
  PCR 3′ end primer
ggccaggaaccatgtgcccctcacctc

SEQ ID NO: 11 - DNA, 30 bp, Artificial Sequence
  PCR 5′ end primer
gaggcacttatctgtttcagcctgtggatc

SEQ ID NO: 12 - DNA, 30 bp, Artificial Sequence
  PCR 3′ end primer
gtgggttagaacggcacgttctcactggag

SEQ ID NO: 13 - DNA, 9276 bp, Pichia pastoris
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattccctagggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 14 - protein, 11 aa, Pichia protease cleavage site
KRIASGVIAPK

SEQ ID NO: 15 - DNA, 1973 bp, Homo sapiens
  CDS (24)...(1952): Nucleotide sequence encoding full-length MTSP20, including MTSP20 protease domains
gcctcgggcggaacctggagataatgggcagcacctgggggagccctggctgggtgcggctcgctctttgcctgacgggcttagtgctctcgctctacgcgctgcacgtgaaggcggcgcgcgcccgggaccgggattaccgcgcgctctgcgacgtgggcaccgccatcagctgttcgcgcgtcttctcctccaggtggggcaggggtttcgggctggtggagcatgtgctgggacaggacagcatcctcaatcaatccaacagcatattcggttgcatcttctacacactacagctattgttaggtcttcaagccgctcagcgtgcctgtggacagcgtggccccggcccccccaagcctcaggagggcaacacagtccctggcgagtggccctggcaggccagtgtgaggaggcaaggagcccacatctgcagcggctccctggtggcagacacctgggtcctcactgctgcccactgctttgaaaaggcagcagcaacagaactgaattcctggtcagtggtcctgggttctctgcagcgtgagggactcagccctggggccgaagaggtgggggtggctgccctgcagttgcccagggcctataaccactacagccagggctcagacctggccctgctgcagctcgcccaccccacgacccacacacccctctgcctgccccagcccgcccatcgcttcccctttggagcctcctgctgggccactggctgggatcaggacaccagtgatgctcctgggaccctacgcaatctgcgcctgcgtctcatcagtcgccccacatgtaactgtatctacaaccagctgcaccagcgacacctgtccaacccggcccggcctgggatgctatgtgggggcccccagcctggggtgcagggcccctgtcagggagattccgggggccctgtgctgtgcctcgagcctgacggacactgggttcaggctggcatcatcagctttgcatcaagctgtgcccaggaggacgctcctgtgctgctgaccaacacagctgctcacagttcctggctgcaggctcgagttcagggggcagctttcctggcccagagcccagagaccccggagatgagtgatgaggacagctgtgtagcctgtggatccttgaggacagcaggtccccaggcaggagcaccctccccatggccctgggaggccaggctgatgcaccagggacagctggcctgtggcggagccctggtgtcagaggaggcggtgctaactgctgcccactgcttcattgggcgccaggccccagaggaatggagcgtagggctggggaccagaccggaggagtggggcctgaagcagctcatcctgcatggagcctacacccaccctgaggggggctacgacatggccctcctgctgctggcccagcctgtgacactgggagccagcctgcggcccctctgcctgccctatcctgaccaccacctgcctgatggggagcgtggctgggttctgggacgggcccgcccaggagcaggcatcagctccctccagacagtgcccgtgaccctcctggggcctagggcctgcagccggctgcatgcagctcctgggggtgatggcagccctattctgccggggatggtgtgtaccagtgctgtgggtgagctgcccagctgtgagggcctgtctggggcaccactggtgcatgaggtgaggggcacatggttcctggccgggctgcacagcttcggagatgcttgccaaggccccgccaggccggcggtcttcaccgcgctccctgcctatgaggactgggtcagcagtttggactggcaggtctacttcgccgaggaaccagagcccgaggctgagcctggaagctgcctggccaacataagtatgtggccccggggcctcctgccaaaccctgcctctccaggacccttctctctccagtgagaacgtgccgttctaacccac

SEQ ID NO: 16 - protein, 642 aa, Homo sapiens
MGSTWGSPGWVRLALCLTGLVLSLYALHVKAARARDRDYRALCDVGTAISCSRVFSSRWGRGFGLVEHVLGQDSILNQSNSIFGCIFYTLQLLLGLQAAQRACGQRGPGPPKPQEGNTVPGEWPWQASVRRQGAHICSGSLVADTWVLTAAHCFEKAAATELNSWSVVLGSLQREGLSPGAEEVGVAALQLPRAYNHYSQGSDLALLQLAHPTTHTPLCLPQPAHRFPFGASCWATGWDQDTSDAPGTLRNLRLRLISRPTCNCIYNQLHQRHLSNPARPGMLCGGPQPGVQGPCQGDSGGPVLCLEPDGHWVQAGIISFASSCAQEDAPVLLTNTAAHSSWLQARVQGAAFLAQSPETPEMSDEDSCVACGSLRTAGPQAGAPSPWPWEARLMHQGQLACGGALVSEEAVLTAAHCFIGRQAPEEWSVGLGTRPEEWGLKQLILHGAYTHPEGGYDMALLLLAQPVTLGASLRPLCLPYPDHHLPDGERGWVLGRARPGAGISSLQTVPVTLLGPRACSRLHAAPGGDGSPILPGMVCTSAVGELPSCEGLSGAPLVHEVRGTWFLAGLHSFGDACQGPARPAVFTALPAYEDWVSSLDWQVYFAEEPEPEAEPGSCLANISMWPRGLLPNPASPGPFSLQ

SEQ ID NO: 17 - protein, 628 aa, Homo Sapien
  ref <300>: Sugen, Inc.
  ref <301>: AUTHORS: Plowman et al.
  ref <309>: DATABASE ENTRY DATE: 2002-01-03
  ref <310>: PATENT DOCUMENT NUMBER: WO 02/000860
MGSTWGSPGWVRLALCLTGLVLSLYALHVKAARARDRDYRALCDVGTAISCSRVFSSRWGRGFGLVEHVLGQDSILNQSNSIFGCIFYTLQLLLGLQAAQRACGQRGPGPPKPQEGNTVPGEWPWQASVRRQGAHICSGSLVADTWVLTAAHCFEKAAATELNSWSVVLGSLQREGLSPGAEEVGVAALQLPRAYNHYSQGSDLALLQLAHPTTHTPLCLPQPAHRFPFGASCWATGWDQDTSDAPGTLRNLRLRLISRPTCNCIYNQLHQRHLSNPARPGMLCGGPQPGVQGPCQGDSGGPVLCLEPDGHWVQAGIISFASSCAQEDAPVLLTNTAAHSSWLQARVQGAAFLAQSPETPEMSDEDSCVACGSLRTAGPQAGAPSPWPWEARLMHQGQLACGGALVSEEAVLTAAHCFIGRQAPEEWSVGLGTRPEEWGLKQLILHGAYTHPEGGYDMALLLLAQPVTLGASLRPLCLPYPDHHLPDGERGWVLGRARPGAGISSLQTVPVTLLGPRACSRLHAAPGGDGSPILPGMVCTSAVGELPSCEGLSGAPLVHEVRGTWFLAGLHSFGDACQGPARPAVFTALPAYEDWVSSLDWQVYFAEEPEPEAEPGSCLANISQPTSC

SEQ ID NO: 18 - DNA, 30 bp, Artificial Sequence
  5′ end primer
gctgcttcaggccccactcctccggtctgg

SEQ ID NO: 19 - DNA, 30 bp, Artificial Sequence
  nested 5′ end primer
cacaggccagctgtccctggtgcatcagcc

SEQ ID NO: 20 - DNA, 30 bp, Artificial Sequence
  5′ end primer
agcacggccggccaatcgccgagtcagagg

SEQ ID NO: 21 - DNA, 29 bp, Artificial Sequence
  sense
gcctcgggcggaacctggagataatgggc

SEQ ID NO: 22 - DNA, 30 bp, Artificial Sequence
  antisense primer
gaccaggaattcagttctgttgctgctgcc

SEQ ID NO: 23 - DNA, 28 bp, Artificial Sequence
  sense primer
aagcctcaggagggcaacacagtccctg

SEQ ID NO: 24 - DNA, 29 bp, Artificial Sequence
  antisense primer
acagctgggcagctcacccacagcactgg

(19) United States
(12) Patent Application Publication
(10) Pub. No.: US 2003/0134298 A1
(43) Pub. Date: Jul. 17, 2003

(54) Nucleic acid molecules encoding a transmembrane serine protease 20, the encoded polypeptides and methods based thereon

(76) Inventors: Edwin L. Madison, San Diego, CA (US); Edgar O. Ong, San Diego, CA (US)

Correspondence Address: Stephanie Seidman, Heller Ehrman White & McAuliffe LLP, 7th Floor, 4350 La Jolla Village Drive, San Diego, CA, 92122-1246, US

(21) Appl. No.: 10/190,030

(22) Filed: Jul. 3, 2002

Related U.S. Application Data
(60) Provisional application No. 60/302,939, filed on Jul. 3, 2001.

(51) Int. Cl. C12Q 1/68; C07H 21/04; C12N 9/64; C12P 21/02; C12N 5/06

(52) U.S. Cl. 435/6; 435/69.1; 435/226; 435/320.1; 435/325; 536/23.2

(57) ABSTRACT

Provided herein are type II transmembrane serine protease 20 (MTSP20) polypeptides. Zymogen and activated forms of these polypeptides as well as single and multi-chain forms of the polypeptides and protease domains are also provided. Methods using the polypeptides for screening, diagnosis and prognosis are also provided.

### RELATED APPLICATIONS

Benefit of priority under 35 U.S.C. §119(e) is claimed to U.S. provisional application Serial No. 60/302,939, filed Jul. 3, 2001, to Edwin L. Madison and Edgar O. Ong, entitled “NUCLEIC ACID MOLECULES ENCODING TRANSMEMBRANE SERINE PROTEASE 20, THE ENCODED PROTEINS AND METHODS BASED THEREON.” The subject matter of this application is incorporated in its entirety by reference thereto.

This application is also related to International PCT application No. ______ (attorney docket no. 24745-1618PC), filed on the same day herewith, entitled “NUCLEIC ACID MOLECULES ENCODING TRANSMEMBRANE SERINE PROTEASE 20, THE ENCODED POLYPEPTIDES AND METHODS BASED THEREON.” The disclosure of the PCT application is herein incorporated by reference in its entirety.

### FIELD OF INVENTION

Nucleic acid molecules that encode proteases and portions thereof, particularly protease domains are provided. Also provided are prognostic, diagnostic and therapeutic methods using the proteases and domains thereof and the encoding nucleic acid molecules.

### BACKGROUND OF THE INVENTION AND OBJECTS THEREOF

Cancer, which is a leading cause of death in the United States, is characterized by an increase in the number of abnormal neoplastic cells, which proliferate to form a tumor mass, the invasion of adjacent tissues by these neoplastic tumor cells, and the generation of malignant cells that metastasize via the blood or lymphatic system to regional lymph nodes and to distant sites. Among the hallmarks of cancer is a breakdown in the communication among tumor cells and their environment. Normal cells do not divide in the absence of stimulatory signals and cease dividing in the presence of inhibitory signals. Growth-stimulatory and growth-inhibitory signals, are routinely exchanged between cells within a tissue. In a cancerous, or neoplastic, state, a cell acquires the ability to “override” these signals and to proliferate under conditions in which normal cells do not grow.

In order to proliferate tumor cells acquire a number of distinct aberrant traits reflecting genetic alterations. The genomes of certain well-studied tumors carry several different independently altered genes, including activated oncogenes and inactivated tumor suppressor genes. Each of these genetic changes appears to be responsible for imparting some of the traits that, in the aggregate, represent the full neoplastic phenotype.

A variety of biochemical factors have been associated with different phases of metastasis. Cell surface receptors for collagen, glycoproteins such as laminin, and proteoglycans, facilitate tumor cell attachment, an important step in invasion and metastases. Attachment triggers the release of degradative enzymes which facilitate the penetration of tumor cells through tissue barriers. Once the tumor cells have entered the target tissue, specific growth factors are required for further proliferation. Tumor invasion and progression involve a complex series of events, in which tumor cells detach from the primary tumor, break down the normal tissue surrounding it, and migrate into a blood or lymphatic vessel to be carried to a distant site. The breaking down of normal tissue barriers is accomplished by the elaboration of specific enzymes that degrade the proteins of the extracellular matrix that make up basement membranes and stromal components of tissues.

A class of extracellular matrix degrading enzymes has been implicated in tumor invasion. Among these are the matrix metalloproteinases (MMP). For example, the production of the matrix metalloproteinase stromelysin is associated with malignant tumors with metastatic potential (see, e.g., McDonnell et al. (1990) Smnrs. in Cancer Biology 1:107-115; McDonnell et al. (1990) Cancer and Metastasis Reviews 9:309-319).

The capacity of cancer cells to metastasize and invade tissue is facilitated by degradation of the basement membrane. Several proteinase enzymes, including the MMPs, have been reported to facilitate the process of invasion of tumor cells. MMPs are reported to enhance degradation of the basement membrane, which thereby permits tumorous cells to invade tissues. For example, two major metalloproteinases having molecular weights of about 70 kDa and 92 kDa appear to enhance ability of tumor cells to metastasize.

Type II Transmembrane Serine Proteases

In addition to the MMPs, serine proteases have been implicated in neoplastic disease progression. Most serine proteases, which are either secreted enzymes or are sequestered in cytoplasmic storage organelles, have roles in blood coagulation, wound healing, digestion, immune responses and tumor invasion and metastasis. A class of cell surface proteins designated type II transmembrane serine proteases, which are membrane-anchored proteins that often include additional extracellular domains, has been identified. As cell surface proteins, they are positioned to play a role in intracellular signal transduction and in mediating cell surface proteolytic events.

Cell surface proteolysis is a mechanism for the generation of biologically active proteins that mediate a variety of cellular functions. Membrane-associated proteases include membrane-type metalloproteinases (MT-MMP), ADAMs (proteases that contain disintegrin-like and metalloproteinase domains) and the transmembrane serine proteases. In mammals, at least 17 members of the transmembrane serine protease family are known, including seven in humans (see, Hooper et al. (2001) J. Biol. Chem. 276:857-860). These include: corin (accession nos. AF133845 and AB013874; see, Yan et al. (1999) J. Bio. Chem. 274:14926-14938; Tomia et al. (1998) J. Biochem. 124:784-789; Uan et al. (2000) Proc. Natl. Acad. Sci. U.S.A. 97:8525-8529); enteropeptidase (also designated enterokinase; accession no. U09860 for the human protein; see, Kitamoto et al. (1995) Biochem. 27: 4562-4568; Yahagi et al. (1996) Biochem. Biophys. Res. Commun. 219:806-812; Kitamoto et al. (1994) Proc. Natl. Acad. Sci. U.S.A. 91:7588-7592; Matsushima et al. (1994) J. Biol. Chem. 269:19976-19982;); human airway trypsin-like protease (HAT; accession no. AB002134; see Yamaoka et al. J. Biol. Chem. 273:11894-11901); MTSP1 and matriptase (also called TADG-15; see SEQ ID Nos. 1 and 2; accession nos. AF133086/AF118224, AF04280022; Takeuchi et al. (1999) Proc. Natl. Acad. Sci. U.S.A. 96:11054-1161; Lin et al. (1999) J. Biol. Chem. 274:18231-18236; Takeuchi et al. (2000) J. Biol. Chem. 275:26333-26342; and Kim et al. (1999) Immunogenetics 49:420-429); hepsin (see, accession nos. Ml 8930, AF030065, X70900; Leytus et al. (1988) Biochem. 27: 11895-11901; Vu et al. (1997) J. Biol. Chem. 272:31315-31320; and Farley et al. (1993) Biochem. Biophys. Acta 1173:350-352; and see, U.S. Pat. No. 5,972,616); TMPRS2 (see, Accession Nos. U75329 and AF113596; Paoloni-Giacobino et al. (1997) Genomics 44:309-320; and Jacquinet et al. (2000) FEBS Lett. 468: 93-100); and TMPRSS4 (see, Accession No. NM 016425; Wallrapp et al. (2000) Cancer 60:2602-2606).

Serine proteases, including transmembrane serine proteases and secreted proteases, have been implicated in processes involved in neoplastic development and progression. While the precise, detailed mechanism by which these proteases promote tumor growth and progression has not been elaborated, serine proteases and inhibitors thereof are involved in the control of many intra- and extracellular physiological processes, including degradative actions in cancer cell invasion, metastatic spread, and neovascularization of tumors, that are involved in tumor progression. It is believed that proteases are involved in the degradation of extracellular matrix (ECM) and contribute to tissue remodeling, and are necessary for cancer invasion and metastasis. The activity and/or expression of some proteases have been shown to correlate with tumor progression and development.

For example, a membrane-type serine protease MTSP1 (also called matriptase; see SEQ ID Nos. 1 and 2 from U.S. Pat. No. 5,972,616; and GenBank Accession No. AF118224; Lin et al. (1999) J. Biol. Chem. 274:18231-18236; U.S. Pat. No. 5,792,616; see, also Takeuchi (1999) Proc. Natl. Acad. Sci. U.S.A. 96:11054-1161) that is expressed in epithelial cancer and normal tissue (Takeucuhi et al. (1999) Proc. Natl. Acad. Sci. USA 96:11054-61) has been identified. Matriptase was originally identified in human breast cancer cells as a major gelatinase (see, U.S. Pat. No. 5,482,848) and was initially believed to be a type of matrix metalloprotease (MMP). It has been proposed that it plays a role in the metastasis of breast cancer. Matriptase also is expressed in a variety of epithelial tissues with high levels of activity and/or expression in the human gastrointestinal tract and the prostate. MTSPs, designated MTSP3, MTSP4, MTSP6 have been described in published International PCT application No. WO 01/57194, based in International PCT application No. PCT/US01/03471.

Prostate-specific antigen (PSA), a kallikrein-like serine protease, degrades extracellular matrix glycoproteins fibronectin and laminin, and has been postulated to facilitate invasion by prostate cancer cells (Webber et al. (1995) Clin. Cancer Res., 1(10:1089-94). Blocking PSA proteolytic activity with PSA-specific monoclonal antibodies results in a dose-dependent decrease in vitro in the invasion of the reconstituted basement membrane Matrigel by LNCaP human prostate carcinoma cells which secrete high levels of PSA.

Hepsin, a cell surface serine protease identified in hepatoma cells, is overexpressed in ovarian cancer (Tanimoto et al. (1997) Cancer Res., 57(14):2884-7). The hepsin transcript appears to be abundant in carcinoma tissue and is almost never expressed in normal adult tissue, including normal ovary. It has been suggested that hepsin is frequently overexpressed in ovarian tumors and therefore can be a candidate protease in the invasive process and growth capacity of ovarian tumor cells.

A serine protease-like gene, designated normal epithelial cell-specific 1 (NES1) (Liu et al., Cancer Res., 56(14):3371-9 (1996)) has been identified. Although expression of the NES1 mRNA is observed in all normal and immortalized nontumorigenic epithelial cell lines, the majority of human breast cancer cell lines show a drastic reduction or a complete lack of its expression. The structural similarity of NES1 to polypeptides known to regulate growth factor activity and a negative correlation of NES1 expression with breast oncogenesis suggest a direct or indirect role for this protease-like gene product in the suppression of tumorigenesis.

Angiogenesis, Modulators and Associated Diseases

Angiogenesis is the generation of new blood vessels from parent microvessels. Controlled and uncontrolled angiogenesis proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Angiogenesis is highly regulated by a system of angiogenic stimulators and inhibitors. Known examples of angiogenesis stimulators include certain growth factors, cytokines, proteins, peptides, carbohydrates and lipids (Norrby, APMIS, 105:417-437 (1997); Polverini, Crit. Rev. Oral. Biol. Med., 6:230-247 (1995)). A variety of endogenous and exogenous angiogenesis inhibitors are known in the art (Jackson et al., FASEB, 11:457-465 (1997); Norrby, APMIS, 105:417-437 (1997); and O'Reilly, Investigational New Drugs, 15:5-13 (1997)).

In adult organisms, capillary endothelial cells divide relatively infrequently. When triggered by appropriate signals, e.g., in response to hormonal signals during menses or following the release of pro-angiogenic mediators sequestered in the extracellular matrix, endothelial cells lining venules will systematically degrade their basement membrane and proximal extracellular matrix, migrate directionally, divide, and organize into new functioning capillaries, within a matter of days (Polverini, Crit. Rev. Oral. Biol. Med., 6:230-247 (1995)). This dramatic amplification of the microvasculature is nevertheless temporary, for as rapidly as the new capillaries are formed, they virtually disappear within a matter of days or weeks, returning the tissue microvasculature to its status quo. It is this feature of transient growth and regression of capillaries that primarily distinguishes physiological angiogenesis from pathological one (Polverini, Crit. Rev. Oral. Biol. Med., 6:230-247 (1995)). In contrast, pathological angiogenesis is caused by a shift in the net balance between stimulators and inhibitors of angiogenesis, e.g., due to the overproduction of normal or aberrant forms of angiogenic mediators, or due to a relative deficiency in inhibitors of this process (Polverini, Crit. Rev. Oral. Biol. Med., 6:230-247 (1995)).

Angiogenesis is essential for normal placental, embryonic, fetal and post-natal development and growth, but almost never occurs physiologically in adulthood except in very specific restricted situations. For example, angiogenesis is normally observed in wound healing, fetal and embryonal development and formation of the corpus luteum, endometrium and placenta. Angiogenesis in the adult is often associated with disease states.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic dependent or angiogenic associated diseases.

The control of angiogenesis is altered in certain disease states and, in many cases, the pathological damage associated with the disease is related to uncontrolled angiogenesis (see generally Norrby, APMIS, 105:417-437 (1997); and O'Reilly, Investigational New Drugs, 15:5-13 (1997)). Thus, angiogenesis is involved in the manifestation or progress of various diseases, for example, various inflammatory diseases, such as rheumatoid arthritis, psoriasis, diabetic retinopathies, certain ocular disorders, including recurrence of pterygii, scarring excimer laser surgery and glaucoma filtering surgery, various disorders of the anterior eye, cardiovascular disorders, chronic inflammatory diseases, wound repair, circulatory disorders, crest syndromes, dermatological disorders (see, e.g., U.S. Pat. Nos. 5,593,990, 5,629,327 and 5,712,291) and notably cancer, including solid neoplasms and vascular tumors. Several lines of direct evidence indicate that angiogenesis is essential for the growth and persistence of solid tumors and their metastases.

Thus, it is clear that angiogenesis plays a major role in the metastasis of cancer and in the pathology of a variety of other disorders. Repressing, eliminating or modulating this activity, should impact the etiology of these diseases and serve as a point of therapeutic intervention. In the disease state, prevention of angiogenesis could avert the damage caused by the invasion of the new microvascular system. Therapies directed at control of the angiogenic processes could lead to the abrogation or mitigation of these diseases.

Hence there is a need to develop therapeutics that target angiogenesis and modulate, particularly, inhibit aberrant or uncontrolled angiogenesis. In addition, transmembrane serine proteases appear to be involved in the etiology and pathogenesis of tumors. There is a need to further elucidate their role in these processes and to identify additional transmembrane proteases. Therefore, it is an object herein, among others, to provide transmembrane serine protease (MTSP) proteins and nucleic acids encoding such MTSP proteases that are involved in the regulation of, or participate in, tumorigenesis and/or carcinogenesis. It is also an object, among others, herein to provide nucleic acids encoding the proteins and polypeptides and also to provide proteins and polypeptides that are involved in the regulation of angiogenesis. Among the objects herein, it is also an object to provide prognostic, diagnostic and therapeutic screening methods using such proteases and the nucleic acids encoding such proteases, and to provide assays for identification of agents that target angiogenesis and modulate, particularly, inhibit aberrant or uncontrolled angiogenesis.

### SUMMARY

Provided herein are polypeptides, including protease domains thereof, designated herein as MTSP20 polypeptides. Protease domains and full-length polypeptides, including the zymogen and activated forms, and uses thereof are also provided. Polypeptides encoded by splice variants are also provided. Also provided are muteins and other derivatives and analogs thereof. Also provided herein are nucleic acids encoding the MTSP20s. The MTSP20s provided herein are also endotheliases, which are a class membrane proteases that are expressed on cells, particularly endothelial cells that participate in angiogenesis.

Protease domains provided herein include, but are not limited to, the single chain region having an N-terminus at the cleavage site for activation of the zymogen, through the C-terminus, or C-terminal truncated portions thereof that exhibit proteolytic activity as a single-chain polypeptide in in vitro proteolysis assays of MTSP20, from a mammal, including a human, that, for example, displays functional activity in tumor cells that is different from its activity non-tumor cells.

Nucleic acid molecules encoding the proteins and protease domains are also provided. Nucleic acid molecules that encode single-chain protease domains or catalytically active portions thereof and multichain forms of proteases domains, and also those that encode the full-length MTSP20 and portions thereof are provided. In an exemplary embodiment, a nucleic acid that encodes an MTSP20 is provided. The nucleic acid molecule includes the sequence of nucleotides set forth in SEQ ID No. 5 or SEQ ID No. 15 or a portion thereof (see, also EXAMPLE 1) that encodes a catalytically active polypeptide or a domain thereof. Nucleic acid encoding a protease domain and upstream nucleic acid is set forth in SEQ ID No. 5; and a protease domain of MTSP20 is set forth in SEQ ID No. 6. An exemplary nucleic acid molecule encoding the full-length polypeptide, which includes two protease domains, is set forth in SEQ ID No. 15; the encoded polypeptide is set forth in SEQ ID No. 16.

Also provided are nucleic acid molecules that encode all or a portion of a catalytically active polypeptide, or a nucleic acid molecule that encodes a protease domain or a larger polypeptide that can include up to the full length polypeptide, and that hybridizes to such MTSP20-encoding nucleic acid along their full-length or along at least about 70%, 80% or 90% of the full-length and encode a protease domain or portion thereof are provided. Hybridization is generally effected under conditions of at least low, generally at least moderate, and often high stringency.

The isolated nucleic acid fragment is DNA, including genomic or cDNA, or is RNA, or can include other components, such as peptide nucleic acid (PNA) or other nucleotide analogs. The isolated nucleic acid can include additional components, such as heterologous or native promoters, and other transcriptional and translational regulatory sequences. These genes may be linked to other genes, such as reporter genes or other indicator genes or genes that encode indicators.

Also provided are isolated nucleic acid molecules that include a sequence of molecules that is complementary to the nucleic acid molecule encoding an MTSP20 or the portion thereof.

Also provided are nucleic acid molecules that hybridize under conditions of at least low stringency, generally moderate stringency, more typically high stringency to the sequence of nucleotides set forth in SEQ ID No. 5 or SEQ ID No. 15 or degenerates thereof. In one embodiment, the isolated nucleic acid fragment hybridizes to a nucleic acid molecule containing the nucleotide sequence set forth in SEQ ID No. 5 or SEQ ID No. 15 (or degenerates thereof) under high stringency conditions. In one embodiment, it contains the sequence of nucleotides set forth in SEQ ID No. 5. A full-length MTSP20 polypeptide includes the sequence of amino acids set forth in SEQ ID No. 6 or SEQ ID No. 16, and is encoded by a sequence of nucleotides set forth in SEQ ID No. 5 or SEQ ID No. 15 or degenerates thereof. Methods for isolating nucleic acid encoding other MTSP20s, including nucleic acid molecules encoding full-length molecules and splice variants and MTSPs from species, such as cows, sheep, goats, pigs, horses, primates, including chimpanzees and gorillas, rodents, dogs, cats and other species of interest, such as domesticated animals, farm and zoo animals are also provided. The nucleic acid molecules provided herein, including those set forth in SEQ ID Nos. 5 and 15 can be used to obtain nucleic acid molecules encoding full-length MTSP20 polypeptides from human sources or from other species, such as by screening or PCR amplification, or other such methods, appropriate libraries using the nucleic acid molecules or selected primers or probes based thereon.

Also provided are fragments thereof or oligonucleotides that can be used as probes or primers and that contain at least about 10, 14, 16 nucleotides, generally less than 1000 or less than or equal to 100, set forth in SEQ ID No. 5 or SEQ ID No. 15 (or the complement thereof); or contain at least about 30 nucleotides (or the complement thereof) or contain oligonucleotides that hybridize along their full-length (or at least about 70, 80 or 90% thereof) to any such fragments or oligonucleotides. The length of the fragments are a function of the purpose for which they are used and/or the complexity of the genome of interest. Generally probes and primers contain less than about 30, 50, 150 or 500 nucleotides.

Also provided are plasmids containing any of the nucleic acid molecules provided herein. Cells containing the plasmids are also provided. Such cells include, but are not limited to, bacterial cells, yeast cells, fungal cells, plant cells, insect cells and animal cells.

Methods of expressing the encoded MTSP20 polypeptide and portions thereof using the cells are also provided, as are cells that express MTSP20 on the cell surface. Such cells are used in methods of identifying candidate therapeutic compounds.

MTSP20, particularly protease domains thereof, can be produced by growing the above-described cells under conditions whereby the MTSP20 is expressed by the cells, and recovering the expressed MTSP20 polypeptide.

Also provided are cells, generally eukaryotic cells, such as mammalian cells and yeast cells, in which the MTSP20 polypeptide is expressed on the surface of the cells. Such cells are used in drug screening assays to identify compounds that modulate an activity of the MTSP20 polypeptide. These assays, including in vitro binding assays, and transcription based assays in which signal transduction is mediated directly or indirectly, such as via activation of pro-growth factors, by the MTSP20 is assessed.

Also provided are peptides that are encoded by such nucleic acid molecules. Included among those polypeptides are an MTSP20 protease domain or a polypeptide with amino acid changes such that the specificity and/or protease activity remains substantially unchanged. In particular, a substantially purified mammalian MTSP20 polypeptide is provided that includes a serine protease catalytic domain and may additionally include other domains. The MTSP20 can form homodimers and can also form heterodimers with some other protein, such as a membrane-bound protein. Also provided is a substantially purified protein including a sequence of amino acids that has at least 60%, 70%, 80%, 81%, 82%, 83%, 84%, 85%, 86%, 87%, 88%, 89%, 90%, 91%, 92%, 93%, 94%, 95%, 96%, 97%, 98%, 99% identity to the MTSP20 where the percentage identity is determined using standard algorithms and gap penalties that maximize the percentage identity. A human MTSP20 polypeptide is exemplified, although other mammalian MTSP20 polypeptides are contemplated. Splice variants of the MTSP20, particularly those with a proteolytically active protease domain, are contemplated herein.

In other embodiments, substantially purified polypeptides that include a protease domain of a MTSP20 polypeptide or a catalytically active portion thereof are provided. Among these are polypeptides that include a sequence of amino acids that has at least 60%, 70%, 80%, 85%, 90%, 95% or 100% sequence identity to SEQ ID No. 6 or SEQ ID No. 16 or to a portion thereof that includes a catalytically active polypeptide.

Also provided are muteins of single chain protease domains of MTSP20 particularly muteins in which the Cys residue in a protease domain that is free (i.e., does not form disulfide linkages with any other Cys residue in a protease domain) is substituted with another amino acid substitution, typically, although not necessarily, with a conservative amino acid substitution or a substitution that does not eliminate an activity, and muteins in which a glycosylation site(s) is eliminated. Hence muteins in which one or more of the Cys residues, particularly, a residue that is paired in the activated two form, but unpaired in a protease domain alone (i.e., the Cys (C₂₁₉) residue position 219 and the Cys (C₄₇₇) residue 477 SEQ ID Nos. 15 and 16 in a protease domain), is/are replaced with any amino acid, typically, although not necessarily, a conservative amino acid residue, such as Ser, are contemplated. Muteins of MTSP20, particularly those in which Cys residues, such as the unpaired Cys in the single chain protease domain, is replaced with another amino acid that does not eliminate an activity, are provided. Muteins in which other conservative or non-conservative amino acid substitutions in which catalytic activity is retained are also contemplated (see, e.g., Table 1, for exemplary amino acid substitutions).

MTSP20 polypeptides, including, but not limited to splice variants thereof, and nucleic acids encoding MTSPs, and domains, derivatives and analogs thereof are provided herein. Single chain protease domains that have an N-terminus functionally equivalent to that generated by activation of the zymogen form of MTSP20 are also provided. The MTSP20 exemplified herein (see SEQ ID No. 16) has a signal peptide (aa 1 to aa 24), a transmembrane domain (aa 82 to aa 99) and two potential N-glycosylation sites ( . . . N₇₇QS . . . and . . . N₆₂₁IS). This MTSP20 has two trypsin-like serine protease domains in tandem, herein after referred to as MTSP20-PD1 (aa 113 to aa 343) and MTSP20-PD2 (aa 375 to aa 596). Both protease domains are characterized by the presence of a protease activation cleavage site at the beginning of the domain and the catalytic triad residues (histidine, aspartate and serine) in 3 highly-conserved regions of the catalytic domain.

MTSP20-PD1 has the following features: a protease cleavage site ( . . . K₁₁₂↓P₁₁₃QEGN . . . , where ↓ indicates the cleavage site); catalytic triad residues (H₁₅₂, D₂₀₃ and S₂₉₉); the following cysteine pairings within the protease domain (C₁₃₇-C₁₅₃, C₂₃₃-C₃₀₅, C₂₆₄-C₂₈₄ and C₂₉₅-C₃₂₄); an unpaired cysteine (C₂₁₉) in the protease domain is predicted to pair with C₁₀₃ so that a multichain form would include amino acid residues from the cleavage site (K₁₁₂ up to and including the C₁₀₃ and optionally back to the N-terminus in the second chain formed upon activation cleavage).

MTSP20-PD2 has the following features: the protease cleavage site ( . . . R₃₇₅↓T₃₇₆AGPQ . . . ); catalytic triad residues (H₄₁₆, D₄₅₇ and S₅₅₃); the following cysteine pairings within the protease domain (C₄₀₁-C₄₁₇, C₅₁₉-C₅₃₉ and C₅₄₉-C₅₇₇); an unpaired cysteine (C₄₇₇) in the protease domain is predicted to pair with C₃₇₁ so that a multichain form would include amino acid R₃₇₅ up to and including the C₃₇₁ and optionally beyond towards the N-terminus in the second chain formed upon activation cleavage. In addition, as noted PD1 can form two chains, so that the entire molecule can include at least three chains.

As noted the protease is provided as a single- or multi-chain form, such as two- or three- or more-chain molecule. Single chain forms of each protease domain and each of the multi-chain forms of a protease domains up to the full length forms are proteolytically active. As noted above, an unpaired Cys in a protease domain pairs with a Cys outside the domain. Upon activation cleavage the bond remains resulting in two or three chains. The size of chain “A”, that is the most N-terminal chain, is a function the starting length of the polypeptide prior to activation cleavage. Similarly, the size of the second so-called chain “B” chain upon activation cleavage of the second protease domain in the polypeptide is a function of the starting length of the polyeptide prior to activation cleavage. Any length polypeptide that includes a protease domain or catalytically active fragments thereof, is contemplated herein. In particular, any polypeptide that includes MTSP20 PD2 is provided, including a full-length MTSP20 polyeptide that includes two protease domains and catatically active portions there of that include amino acids 624-642 or portions thereof. Also provided are the isolated protease domain PD1 as an isolated single cahin protease and as a two chain activated form that includes up to amino acid 343 (SEQ ID NO. 16), particularly amino acids 113-343 as a single chain isolated polypeptide. Also provided are polyeptides encoded by nucleic acid molecules that are encoded by nucleic acid molecules that hybridize under specified conditions as described herein to the exemplified nucleic acid molecule and polypeptides that are homologous as described herein to the the exemplified polypeptide.

MTSPs are expressed or are activated in certain tumor or cancer cells such as lung, prostate, colon and breast, ovarian, pancreatic, lung in other tumors. MTSP20 is of interest because it is expressed or is active in tumor cells. In particular, it is shown herein, that MTSP20 is, for example, expressed in esophageal tumor tissues, in lung carcinoma, colon, cervical, leukemia and other cell lines as well as in certain normal cells and tissues (see e.g., EXAMPLES for tissue-specific expression profile). The level of activated MTSP20 can be diagnostic of prostate, uterine, lung, esophagus or colon cancer, or leukemia or other cancer. The expression and/or activation of MTSP20 on or in the vicinity of a cell or in a bodily fluid in a subject can be a marker for breast, prostate, lung, colon and other cancers.

Hence the MTSPs provided herein can serve as diagnostic markers for certain tumors. In certain embodiments, the MTSP20 polypeptide or portions thereof, particularly a protease domain thereof, is detectable in a body fluid at a level or in a form that differs from its level or in a form in body fluids in a subject not having a tumor. In other embodiments, the polypeptide is present in a tumor; and a substrate or cofactor for the polypeptide is expressed at levels that differ from its level of expression in a non-tumor cell in the same type of tissue. In other embodiments, the level of expression and/or activity of the MTSP20 polypeptide in tumor cells differs from its level of expression and/or activity in non-tumor cells. In other embodiments, the MTSP20 is present in a tumor; and a substrate or cofactor for the MTSP20 is expressed at levels that differ from its level of expression in a non-tumor cell in the same type of tissue.

Assays for identifying effectors, such as compounds, including small molecules, and conditions, such pH, temperature and ionic strength, that modulate the activation, expression or activity of MTSP20 are also provided herein. In exemplary assays, the effects of test compounds on the ability of a protease domain of MTSP20 to proteolytically cleave a known substrate, typically a fluorescently, chromogenically or otherwise detectably labeled substrate, are assessed. Agents, generally compounds, particularly small molecules, that modulate an activity of a protease domain are candidate compounds for modulating an activity of an MTSP20. A protease domains can also be used to produce protease-specific antibodies.

Also provided are methods for screening for compounds that modulate an activity of MTSP20. The compounds are identified by contacting them with an MTSP20 or protease domain thereof and a substrate for the MTSP20. A change in the amount of substrate cleaved in the presence of the compounds compared to that in the absence of the compound indicates that the compound modulates an activity of an MTSP20. Such compounds are selected for further analyses or for use to modulate the activity of an MTSP20, such as inhibitors or agonists. The compounds can also be identified by contacting the substrates with a cell that expresses an MTSP20 or an extracellular domain or proteolytically active portion of thereof.

Computer-based screening methods are also provided. In these methods, these interactions between test compounds computer simimulated MTSP20 polypeptides are assessed, such as by computational docking or binding studies. Test compounds predicted to bind or otherwise interact with MTSP20 polypeptides are selected as drug candidates. Further characterization and study, such in vitro assays, can be performed.

Also provided herein are methods of modulating an activity of an MTSP20 and screening for compounds that modulate, including inhibit, antagonize, agonize or otherwise alter an activity of the MTSP20. Of particular interest is an extracellular domain of MTSP20 that includes a proteolytic (catalytic) portion of the protein.

Additionally provided herein are antibodies that specifically bind to single and/or multi-chain forms of MTSP20, cells, combinations, kits and articles of manufacture that contain the antibodies. Antibodies that specifically bind to the MTSP20, particularly a single-chain protease domain, a two-chain form of a protease domain, the zymogen and multi-chain activated forms of an MTSP20 and other fragments thereof. Neutralizing antibodies that inhibit a biological activity, particularly a protease activity are also provided. In particular, antibodies that specifically bind to an MTSP20 that includes two protease domains, particularly that includes amino acids that correspond to amino acids 624-642 of SEQ ID No. 16, with at least 10-fold, generally at least 100-fold, greater affinity than to an MTSP of SEQ ID No. 17 are provided.

Further provided herein are prognostic, diagnostic, therapeutic screening methods using MTSP20 and the nucleic acids encoding MTSP20. In particular, the prognostic, diagnostic and therapeutic screening methods are used for preventing, treating, or for finding agents useful in preventing or treating, tumors or cancers such as lung carcinoma, colon adenocarcinoma and ovarian carcinoma.

Also provided herein are modulators of an activity of MTSP20, especially the modulators obtained according to the screening methods provide herein. Such modulators can have use in treating cancerous conditions.

Methods of diagnosing a disease or disorder characterized by detecting an aberrant level of an MTSP20 in a subject is provided. The method can be practiced by measuring the level of the DNA, RNA, protein or a functional activity of an MTSP20. An increase or decrease in the level of the DNA, RNA, protein or functional activity of the MTSP, relative to the level of the DNA, RNA, protein or functional activity found in an analogous sample not having the disease or disorder (or other suitable control) is indicative of the presence of the disease or disorder in the subject or other relative any other suitable control.

Also provided are methods of identifying a compound that binds to the single-chain and/or multi-chains form of MTSP20, by contacting a test compound with these forms; determining to which form the compound binds; and if it binds to a form of MTSP20, further determining whether the compound has at least one of the following properties:

(i) inhibits activation of a zymogen form of a protease domain of an MTSP20;

(ii) inhibits activity of catalytically active form (i.e. a mutlichain or single-chain form of a protease domain); and

(iii) inhibits dimerization of the protein.

The forms can be full length or truncated forms, including but not limited to, a protease domains resulting from cleavage at one or both of the activation cleavage sites; or from expression of a protease domain or catalytically active portions thereof. Methods for identifying compounds that bind to or interact with the MTSP20, particularly, one or both of a protease domains thereof, are provided. The identified compounds are candidates or leads for identification of compounds for treatments of tumors and other disorders and diseases involving aberrant angiogenesis and/or loss of appropriate growth regulation.

Pharmaceutical composition containing a protease domain and/or full-length or other domain of an MTSP20 polypeptide are provided herein in a pharmaceutically acceptable carrier or excipient are provided herein.

Also provided are articles of manufacture that contain MTSP20 polypeptides and/or a protease domain or protease domains of an MTSP20 in single chain forms or activated forms. The articles contain a) packaging material; b) the polypeptide (or encoding nucleic acid), particularly the single chain protease domain thereof; and c) a label indicating that the article is for using in assays for identifying modulators of the activities of an MTSP20 polypeptide.

Conjugates containing a) an MTSP20 polypeptide or protease domain in a single or multi-chain form; and b) a targeting agent linked to the MTSP directly or via a linker, wherein the agent facilitates: i) affinity isolation or purification of the conjugate; ii) attachment of the conjugate to a surface; iii) detection of the conjugate; or iv) targeted delivery to a selected tissue or cell, are provided herein. The conjugate can contain a plurality of agents linked thereto. The conjugate can be a chemical conjugate; and it can be a fusion protein. The targeting agent can be a protein or peptide fragment. The protein or peptide fragment can include a protein binding sequence, a nucleic acid binding sequence, a lipid binding sequence, a polysaccharide binding sequence, or a metal binding sequence.

Combinations, kits and articles of manufacture containing the MTSP20 polypeptides, domains thereof, or encoding nucleic acids are also provided herein. For example, combinations are provided herein. The combination can include: a) an inhibitor of an activity of an MTSP20; and b) an anti-cancer treatment or agent. The MTSP inhibitor and the anti-cancer agent can be formulated in a single pharmaceutical composition or each is formulated in a separate pharmaceutical composition. The MTSP20 inhibitor can be an antibody or a fragment or binding portion thereof made against the MTSP20, such as an antibody that specifically binds to a protease domain, an inhibitor of MTSP20 production, or an inhibitor of MTSP20 membrane-localization or an inhibitor of MTSP20 activation. Other MTSP20 inhibitors include, but are not limited to, an antisense nucleic acid or double-stranded RNA (dsRNA), such as RNAi, encoding the MTSP20, particularly a portion of a protease domain; a nucleic acid encoding at least a portion of a gene encoding the MTSP20 with a heterologous nucleotide sequence inserted therein such that the heterologous sequence inactivates the biological activity of MTSP20 or the gene encoding it. For example, the portion of the gene encoding the MTSP20 can flank the heterologous sequence to promote homologous recombination with a genomic gene encoding the MTSP20.

In another embodiment, combinations are provided herein that include an inhibitor of an MTSP20 or an inhibitor of a protease activity thereof, and another anti-angiogenic treatment or agent. The MTSP20 inhibitor and the anti-angiogenic agent can be formulated in a single pharmaceutical composition or each can be formulated in a separate pharmaceutical composition. Kits containing the combinations are provided.

Also provided are transgenic non-human animals bearing inactivated genes encoding the MTSP and bearing the genes encoding the MTSP20 under non-native promotor control are provided. Such animals are useful in animal models of tumor initiation, growth and/or progression models. Transgenic non-human animals containing heterolgous nucleic acid MTSP20 under native, non-native promotor control or on an exogenous element, such as a plasmid or artificial chromosome, are additionally provided herein. In particular, recombinant non-human animals are provided herein, where the gene of an MTSP20 is under control of a promoter that is not the native promoter of the gene or that is not the native promoter of the gene in the non-human animal or where the nucleic acid encoding the MTSP20 is heterologous to the non-human animal and the promoter is the native or a non-native promoter or the MTSP20 is on an extrachromosomal element, such as a plasmid or artificial chromosome. Recombinant and transgenic animals can be produced by homologous recombination and non-homologous recombination methods.

Methods of gene therapy are provided. Such methods can be effected by administering in vivo or ex vivo an inactivating form of the MTSP20 or by administering an MTSP-encoding nucleic acid molecule.

Also provided are methods for treating or preventing a tumor or cancer in a mammal by administering to a mammal an effective amount of an inhibitor of an MTSP20, whereby the tumor or cancer is treated or prevented. The MTSP20 inhibitor used in treatment or for prophylaxis is administered with a pharmaceutically acceptable carrier or excipient. The mammal treated can be a human. The treatment or prevention method can additionally include administering an anti-cancer treatment or agent simultaneously with, subsequent to, or before administration of the MTSP20 inhibitor.

Also provided are methods of treatment of tumors by administering a prodrug that is activated by MTSP20 that is expressed or active in tumor cells and/or angiogenic endothelial cells, particularly those in which its functional activity in tumor cells is greater than in non-tumor cells or in which its functional activity in angiogenic endothelial is greater than in quiescent endothelial cells. The prodrug is administered and, upon administration, active MTSP20 expressed on cells cleaves the prodrug and releases active drug in the vicinity of the tumor cells. The active anti-cancer drug accumulates in the vicinity of the tumor. This is useful since the MTSP20 is expressed or active in tumor cells and/or angiogenic endothelial cells and also is useful where the MTSP20 is expressed or active in greater quantity, higher level or predominantly in tumor cells and/or angiogenic endothelial cells compared with other cells.

Also provided are methods of diagnosing the presence of a pre-malignant lesion, a malignancy, or other pathologic condition in a subject, by obtaining a biological sample from the subject; and exposing it to a detectable agent that binds to a multi-chain and/or single-chain form of MTSP20, where the pathological condition is characterized by the presence or absence of the multi-chain and/or single-chain form.

Prognostic, diagnostic and therapeutic screening methods for preventing or treating, or for finding agents useful in preventing or treating, diseases or disorders associated with an aberrant level of angiogenesis and/or cell proliferation also are provided.

Methods for screening for compounds that modulate an activity of the MTSP20 are provided. In vitro assays in which the candidate compounds are identified by contacting them with the MTSP20 or a protease domain thereof and a substrate for the MTSP20 are provided. A change in the amount of substrate cleaved in the presence of the compounds compared to the absence of a candidate compound indicates that the compound modulates an activity of an MTSP20 or a protease domain or domains thereof. Such candidate compounds are selected for further analyses or for use to modulate an activity of an MTSP20, such as, as inhibitors or agonists. The in vitro assays can be performed in liquid phase or on solid phase substrates by linking the MTSP20 or protease domain thereof directly or via a linker to a solid support. Cell-based screening assays are also provided. The compounds can also be identified by contacting the substrates with a cell that expresses the MTSP20 or a proteolytically active portion thereof.

In one embodiment, the method for identifying a modulator of an activity of an MTSP20 includes: a) contacting an MTSP20 with a substrate therefor, and detecting the proteolysis of the substrate, whereby an activity of the MTSP20 is assessed; b) contacting the MTSP20 with a substrate thereof in the presence of a test substance, and detecting the proteolysis of the substrate, whereby an activity of the MTSP20 is assessed; and c) comparing an activity of the MTSP20 assessed in steps a) and b), whereby a difference in activity measured in step a) from an activity measured in step b) indicates that the test substance modulates an activity of an MTSP20. A plurality of the test substances can be screened simultaneously in the above screening method.

In another embodiment, the MTSP20 to be screened is isolated from a target cell and the test substance is a therapeutic compound. A difference in the MTSP20 activity measured in the presence and in the absence of the test substance indicates that the target cell responds to the therapeutic compound.

Diagnostic methods are also provided. For example, detection of protease domain(s) in the blood or other body fluid can be indicative of cancer, particularly metastatic cancer.

Methods of inhibiting tumor invasion or metastasis or treating a malignant or pre-malignant condition by administering an agent that inhibits activation of a zymogen form of MTSP20 of a protease domain of an MTSP20 or an activity of an activated form are provided. The conditions include, but are not limited to, a condition, such as a tumor, of the breast, cervix, prostate, lung, ovary or colon.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such indentifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

As used herein, serine protease refers to a diverse family of proteases wherein a serine residue is involved in the hydrolysis of proteins or peptides. The serine residue can be part of the catalytic triad mechanism, which includes a serine, a histidine and an aspartic acid in the catalysis, or be part of the hydroxyl/ε-amine or hydroxyl/α-amine catalytic dyad mechanism, which involves a serine and a lysine in the catalysis. Of particular interest are serine proteases (SPs) of mammalian, including human, origin. Those of skill in this art recognize that, in general, single amino acid substitutions in non-essential regions of a polypeptide do not substantially alter biological activity (see, e.g., Watson et al. (1987) Molecular Biology of the Gene, 4th Edition, The Benjamin/Cummings Pub. co., p.224).

As used herein, “transmembrane serine protease (MTSP)” refers to a family of transmembrane serine proteases that share common structural features as described herein (see, also Hooper et al. (2001) J. Biol. Chem.276:857-860). Thus, reference, for example, to “MTSP” encompasses all proteins encoded by the MTSP gene family, including but are not limited to: MTSP3, MTSP4, MTSP6, MTSP7 or an equivalent molecule obtained from any other source or that has been prepared synthetically or that exhibits the same activity. Other MTSPs include, but are not limited to, corin, enteropeptidase, human airway trypsin-like protease (HAT), MTSP1, TMPRSS2 and TMPRSS4. Sequences of encoding nucleic acid molecules and the encoded amino acid sequences of exemplary MTSPs and/or domains thereof are set forth, for example in U.S. application Ser. No. 09/776,191 (SEQ ID Nos. 1-12, 49, 50 and 61-72 therein, published as International PCT application No. WO 01/57194). The term also encompass MTSPs with amino acid substitutions that do not substantially alter activity of each member and also encompasses splice variants thereof. Suitable substitutions, including, although not necessarily, conservative substitutions of amino acids, are known to those of skill in this art and can be made without eliminating the biological activity, such as the catalytic activity, of the resulting molecule.

As used herein an MTSP20, whenever referenced herein, includes at least one or all of or any combination of:

a polypeptide encoded by the sequence of nucleotides set forth in SEQ ID No. 5 or SEQ ID No. 15 or by a sequence of nucleotides that includes nucleotides that encode the sequence of amino acids set forth in SEQ ID No. 6 or SEQ ID No. 16;

a polypeptide encoded by a sequence of nucleotides that hybridizes under conditions of low, moderate or high stringency to the sequence of nucleotides set forth in is set forth as SEQ ID No. 5 or SEQ ID No. 15;

a polypeptide that includes the sequence of amino acids set forth in SEQ ID No. 6 or SEQ ID No. 16 or a catalytically active portion thereof, including PD1 and/or PD2 and catalytically active portions thereof;

a polypeptide that includes a sequence of amino acids having at least about 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 6 or SEQ ID No. 16; and/or

a polypeptide encoded by a splice variant of the MTSP20 that includes the sequence of amino acids set forth in SEQ ID No. 6 or SEQ ID No. 16, particularly residues that include residues 624-642 (or a portion thereof) of SEQ ID No. 16.

In particular, MTSP20 polypeptides, with protease domains as indicated in SEQ ID Nos. 5, 6, 15 and 16 are provided. The polypeptide is a single or multi-chain polypeptide. Each protease domain can form a two chain polypeptide upon activation cleavage and can be employed as a portion of the full length molecule or as a full-length molecule. The full-length molecule can be two chains, three chains or, if there are additional cleavages, more chains. Where there are two chains, the shortest chain (most N-terminal chain) is called the A chain, the next chain the B chain and the third chain the C chain (when both protease domains are activated). Where a single protease domain or fragment thereof or longer form thereof activated and isolated, the two chains are referred to as the A chain and B chain, respectively.

Smaller portions thereof that retain protease activity are also provided. Protease domains from MTSPs vary in size and constitution, including insertions and deletions in surface loops. They retain conserved structure, including at least one of the active site triad, primary specificity pocket, oxyanion hole and/or other features of serine protease domains of proteases. Thus, for purposes herein, a protease domain is a portion of an MTSP, as defined herein, and is homologous to a domain of other MTSPs, such as corin, enteropeptidase, human airway trypsin-like protease (HAT), MTSP1, TMPRSS2, and TMPRSS4, which have been previously identified; it was not recognized, however, that an isolated single chain form of a protease domain could function proteolytically in in vitro assays. As with the larger class of enzymes of the chymotrypsin (S1) fold (see, e.g., Internet accessible MEROPS data base), the MTSPs protease domains share a high degree of amino acid sequence identity. The His, Asp and Ser residues necessary for activity are present in conserved motifs. The activation site, which results in the N-terminus of the second chain in a multi-chain form is located in a conserved motif and/or location and can be identified. In the exemplified MTSP20, there are two activation cleavage sites and two protease domains.

The MTSP20 can be from any animal, particularly a mammal, and includes but are not limited to, primates, including humans, rodents, fowl, ruminants, pigs and other animals. The full-length zymogen or multi-chain activated forms are contemplated or any domain thereof, including protease domains, which can be two-chain activated forms, or a single chain forms.

As used herein, a “protease domain of an MTSP” refers to an extracellular protease domain of an MTSP that exhibits proteolytic activity and shares homology and structural features with the chymotrypsin/trypsin family protease domains. Hence it is at least the minimal portion of the domain that exhibits proteolytic activity as assessed by standard in vitro assays. Contemplated herein are such protease domains and catalytically active portions thereof. Also provided are truncated forms of a protease domain that include the smallest fragment thereof that acts catalytically as a single chain form.

A protease domain of an MTSP20, whenever referenced herein, includes at least one or all of or any combination of or a catalytically active portion of:

a) a polypeptide that includes the sequence of amino acids set forth in SEQ ID No. 6 and those in which residue 247 is lie as the only MTSP20 residues or a catalytically active portion thereof;

b) a polypeptide that includes the sequence of amino acids set forth as residues 376-642 in SEQ ID No. 16;

c) a polypeptide encoded by a sequence of nucleotides that hybridizes under conditions of low, moderate or high stringency to the sequence of nucleotides set forth in SEQ ID No. 5 or to SEQ ID No. 15 or to the sequence of amino acids that encodes at least residues 624-642 or SEQ ID No. 16;

d) a polypeptide that includes the sequence of amino acids set forth in SEQ ID No. 6 and at least residues 376-624, generally 376-642, of SEQ ID No. 16 or a catalytically active portion thereof;

e) a polypeptide that includes a sequence of amino acids having at least about 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids of a)-d); and/or

f) a protease domain of a polypeptide encoded by a splice variant of a sequence of nucleotides that encodes an MTSP20 of any of a)-e).

Protease domains of MTSPs vary in size and constitution, including insertions and deletions in surface loops. They retain conserved structure, including at least one of the active site triad, primary specificity pocket, oxyanion hole and/or other features of serine protease domains of proteases. Thus, for purposes herein, a protease domain is a portion of an MTSP, as defined herein, and is homologous to a domain of other MTSP. As with the larger class of enzymes of the chymotrypsin (S1) fold (see, e.g., Internet accessible MEROPS data base), the MTSP protease domains share a high degree of amino acid sequence identity. The His, Asp and Ser residues necessary for activity are present in conserved motifs. The activation site, whose cleavage creates the N-terminus of a protease domain in multi-chain forms is located in a conserved motif and/or location and readily can be identified.

By active form is meant a form active in vivo and/or in vitro. As described herein, a protease domain also can exist as a two-chain or multi-chain form. At least in vitro, the single chain forms of the SPs and the catalytic domains or proteolytically active portions thereof (typically C-terminal truncations) exhibit protease activity. Hence provided herein are isolated single chain forms of protease domains of MTSP20s and their use in in vitro drug screening assays for identification of agents that modulate an activity thereof.

As used herein, reference to “A” chain, “B” chain and “C” chain refers to the multiple chains in an MTPS upon activation cleavage of one or more protease domain. The full-length molecule can be two chains, three chains or, if there are additional cleavages, more chains. Where there are two chains, the shortest chain (most N-terminal chain) is called the A chain, the next chain the B chain and the third chain the C chain (when both protease domains are activated). Where a single protease domain or fragment thereof or longer form thereof activated and isolated, the two chains are referred to as the A chain and B chain, respectively.

As used herein, the catalytically active domain of an MTSP refers to a protease domain. Reference to a protease domain of an MTSP generally refers to a single chain form of the domain. If a multi-chain form is intended, it is so-specified.

As used herein, activation cleavage refers to the cleavage of the protease at an N-terminus of a protease domain. By virtue of the Cys-Cys pairing between a Cys outside a protease domain and a Cys in the protease domain (in this instance Cys₂₁₉ and Cys₄₇₇ SEQ ID No. 16), upon cleavage of one activation site the resulting polypeptide has two chains (“A” chain and the “B” chain, which is the protease domain); upon cleavage of both domains the resulting polpeptide can have three chains (an A, B and C chain). Cleavage can be effected by another protease or autocatalytically.

As used herein, a two-chain form of a protease domain refers to a two-chain form that is formed upon activation cleavage of one cleavage site in the protease in which there is Cys pairing between Cys outside a protease domain and an unpaired Cys in a protease domain, which links a protease domain to a remaining N-terminal portion of the polypeptide. It also includes forms in which the “remaining protion of the polypeptide”, is shortened and includes the Cys outside a protease domain. For example, in PD1, cysteine (C₂₁₉) in the protease domain is predicted to pair with C₁₀₃ outside the protease domain, so that a two chain form includes C₁₀₃ up to and include the N-terminus of the MTSP20. In PD2, the unpaired cysteine (C₄₇₇) in the protease domain is predicted to pair with C₃₇₁, so that a two chain form includes C₁₀₃ up to and include the N-terminus of the MTSP20. If both protease domains are included, there resulting polypeptide contains three chains and includes up to the N-terminus of the MTSP20. N-terminal and C-terminal truncated forms are contemplated, such that the resulting polypeptide exhibits proteolytic activity. Also contemplated are forms that include only one of protease domains, in which case the N-terminus of the PD2 containing polypeptide will not necessarily be coincident with the N-terminus of the polypeptide, but will be up to the N-terminus of the polypeptide prior to activation cleavage.

MTSP20s of interest include those that are activated, active and/or expressed in tumor cells (or angiogenic endothelial cells) different from, typically higher, those in non-tumor (or endothelial) cells; and those from cells in which substrates therefor differ in tumor cells from non-tumor cells or differ with respect to the substrates, co-factors or receptors, or otherwise alter an activity or specificity of an MTSP20.

As used herein, a human protein is one encoded by nucleic acid, such as DNA, present in the genome of a human, including all allelic variants and conservative variations as long as they are not variants found in other mammals.

As used herein, a “nucleic acid encoding a protease domain or catalytically active portion of a SP” shall be construed as referring to a nucleic acid encoding only the recited single chain protease domain or active portion thereof, and not the other contiguous portions of the SP as a continuous sequence.

As used herein, catalytic activity refers to an activity of the MTSP as a protease. Function of the MTSP refers to its function in tumor biology, including promotion of or involvement in initiation, growth or progression of tumors, and also roles in signal transduction. Catalytic activity refers to the activity of an MPSP as a protease as assessed in in vitro proteolytic assays that detect proteolysis of a selected substrate.

As used herein, a CUB domain is a motif that mediates protein-protein interactions in complement components C1r/C1s and has also been identified in various proteins involved in developmental processes.

As used herein, “LDLR” refers to a low density lipoprotein receptor domain, which mediates binding to an LDL receptor.

As used herein, a zymogen is an inactive precursor of a proteolytic enzyme or proteolytic domain thereo. Such precursors are generally larger, although not necessarily larger than the active form. With reference to serine proteases, zymogens are converted to active enzymes by specific cleavage, including catalytic and autocatalytic cleavage, or by binding of an activating co-factor, which generates an active enzyme. A zymogen, thus, is an enzymatically inactive protein that is converted to a proteolytic enzyme by the action of an activator.

As used herein, “disease or disorder” refers to a pathological condition in an organism resulting from, e.g., infection or genetic defect, and characterized by identifiable symptoms.

As used herein, neoplasm (neoplasia) refers to abnormal new growth, and thus means the same as tumor, which can be benign or malignant. Unlike hyperplasia, neoplastic proliferation persists even in the absence of the original stimulus.

As used herein, neoplastic disease refers to any disorder involving cancer, including tumor development, growth, metastasis and progression.

As used herein, cancer refers to a general term for diseases caused by any type of malignant tumor.

As used herein, malignant, as applies to tumors, refers to primary tumors that have the capacity of metastasis with loss of growth control and positional control.

As used herein, an anti-cancer agent (used interchangeably with “anti-tumor or anti-neoplastic agent”) refers to any agents used in the anti-cancer treatment. These include any agents, when used alone or in combination with other compounds, that can alleviate, reduce, ameliorate, prevent, or place or maintain in a state of remission of clinical symptoms or diagnostic markers associated with neoplastic disease, tumor and cancer, and can be used in methods, combinations and compositions provided herein. Non-limiting examples of anti-neoplastic agents include anti-angiogenic agents, alkylating agents, antimetabolites, certain natural products, platinum coordination complexes, anthracenediones, substituted ureas, methylhydrazine derivatives, adrenocortical suppressants, certain hormones, antagonists and anti-cancer polysaccharides.

As used herein, a splice variant refers to a variant produced by differential processing of a primary transcript of genomic nucleic acid, such as DNA, that results in more than one type of mRNA. Splice variants of SPs are provided herein.

As used herein, endotheliase refers to a mammalian protein, including a human protein, that has a transmembrane domain and is expressed on the surface of endothelial cells and includes a protease domain, particularly an extracellular protease domain, that is typically a serine protease. Thus, reference, for example, to endotheliase encompasses all proteins encoded by the endotheliase gene family, or an equivalent molecule obtained from any other source or that has been prepared synthetically or that exhibits the same activity. The endotheliase gene family are transmembrane proteases expressed in endothelial cells. These proteases include serine proteases. When more particularity is need, with respect to the protein provided herien, it refers to proteins that have these features and also include a protease domain that exhibits sequence homology to a protease domain of the endotheliase exemplified herein. Sequence homology means sequence identity along its length when aligned to maximize identity of at least about 25%, 40%, 60%, 80%, 90% or greater number of residues. Sequence homology also is assessed by determining whether the encoding sequences of nucleic acids hybridize under conditions of at least moderate, or for more closely related proteins, high stringency to the nucleic acid molecules provided herein or to those that encode the same proteins but differ in sequence by virtue of the degeneracy of the genetic code. In addition, endotheliases encompass endotheliases with amino acid substitutions, including those set forth in Table 1, that do not substantially alter its proteolytic activity. Suitable substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e+., Watson et al. Molecular Biology of the Gene, 4th Edition, 1987, The Benjamin/Cummings Pub. co., p.224). Also included within the definition, is the catalytically active fragment of an endotheliase.

As used herein, a protease domain of an endotheliase refers to a polypeptide portion of the endotheliase that exhibits protease activity. A protease domain is a polypeptide that includes at least the minimum number of amino acids, generally more than 50 or 100, required for protease activity. Protease activity may be assessed empirically, such as by testing the polypeptide for its ability to act as a protease. Assays, such as in the assays described in the EXAMPLES may be used. Furthermore, since proteases, particularly serine proteases, have characteristic structures and sequences or motifs, a protease domain can readily identified by such structure and sequence or motif.

As used herein, a catalytically active portion of a protease domain of endotheliase refers to a protease domain or portion thereof that exhibits serine proteolytic activity. Hence it is at least the minimal portion or portions where, as in this intance, there are a plurality of protease domains, of an extracellular domain that exhibits proteolytic activity as assessed by standard assays. Smaller portions thereof that retain protease activity are contemplated. Protease domains vary in size and constitution, including insertions and deletions in surface loops. Such domains exhibit conserved structure, including at least one structural feature, such as the active site triad, primary specificity pocket, oxyanion hole and/or other features of serine protease domains of proteases. Thus, for purposes herein, a protease domain is a portion of an endotheliase, as defined herein, that is homologous in terms of structural features and retention of sequence of similarity or homology the protease domain of chymotrypsin or trypsin.

As used herein, angiogenesis is intended to broadly encompass the totality of processes directly or indirectly involved in the establishment and maintenance of new vasculature (neovascularization), including, but not limited to, neovascularization associated with tumors.

As used herein, anti-angiogenic treatment or agent refers to any therapeutic regimen and compound, when used alone or in combination with other treatment or compounds, that can alleviate, reduce, ameliorate, prevent, or place or maintain in a state of remission of clinical symptoms or diagnostic markers associated with undesired and/or uncontrolled angiogenesis. Thus, for purposes herein an anti-angiogenic agent refers to an agent that inhibits the establishment or maintenance of vasculature. Such agents include, but are not limited to, anti-tumor agents, and agents for treatments of other disorders associated with undesirable angiogenesis, such as diabetic retinopathies, restenosis, hyperproliferative disorders and others.

As used herein, non-anti-angiogenic anti-tumor agents refer to anti-tumor agents that do not act primarily by inhibiting angiogenesis.

As used herein, pro-angiogenic agents are agents that promote the establishment or maintenance of the vasculature. Such agents include agents for treating cardiovascular disorders, including heart attacks and strokes.

As used herein, undesired and/or uncontrolled angiogenesis refers to pathological angiogenesis wherein the influence of angiogenesis stimulators outweighs the influence of angiogenesis inhibitors. As used herein, deficient angiogenesis refers to pathological angiogenesis associated with disorders where there is a defect in normal angiogenesis resulting in aberrant angiogenesis or an absence or substantial reduction in angiogenesis.

As used herein, a protease domain of an SP protein refers to a domain of an SP that exhibits proteolytic activity. Hence it is at least a minimal portion of the protein that exhibits proteolytic activity as assessed by standard assays in vitro. It refers, herein, to a single chain form and also the multi-chain activated forms. Exemplary protease domains include at least a sufficient portion of sequences of amino acids set forth in SEQ ID No. 6 (encoded by nucleotides in SEQ ID No. 5) to exhibit protease activity.

Also contemplated are nucleic acid molecules that encode a polypeptide that has proteolytic activity in an in vitro proteolysis assay and that have at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the full-length or a protease domain of an MTSP20 polypeptide or other domain thereof, or that hybridize along their full-length or along at least about 70%, 80% or 90% of the full-length to a nucleic acids that encode a protease domain or other domain, particularly under conditions of moderate, generally high, stringency.

For protease domains, residues at the N-terminus can be critical for activity. It is shown herein that a protease domain of the single chain form of the MTSP20 protease is catalytically active. Hence, a protease domain generally requires the N-terminal amino acids thereof for activity; the C-terminus portion can be truncated. The amount that can be removed can be determined empirically by testing the polypeptide for protease activity in an in vitro assay that assesses catalytic cleavage.

Hence smaller portions of protease domains, particularly single chain domains, thereof that retain protease activity are contemplated. Such smaller versions generally are C-terminal truncated versions of the protease domains. Such domains exhibit conserved structure, including at least one structural feature, such as the active site triad, primary specificity pocket, oxyanion hole and/or other features of serine protease domains of proteases. Thus, for purposes herein, a protease domain is a single chain portion of an MTSP20 domain, as defined herein, but is homologous in its structural features and retention of sequence of similarity or homology to the protease domain of chymotrypsin or trypsin. A protease domain polypeptide can exhibit proteolytic activity as a single chain.

As used herein, “homologous” means about greater than 25% nucleic acid sequence identity, such as 25% 40%, 60%, 70%, 80%, 90% or 95%. If necessary the percentage homology will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule.

Whether any two nucleic acid molecules have nucleotide sequences that are at least, for example, 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(1):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, the term “at least 90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions. At the level of homologies or identities above about 85-90%, the result should be independent of the program and gap parameters set; such high levels of identity can be assessed readily, often without relying on software.

As used herein, primer refers to an oligonucleotide containing two or more deoxyribonucleotides or ribonucleotides, typically more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, animals include any animal, such as, but not limited to, goats, cows, deer, sheep, rodents, pigs and humans. Non-human animals exclude humans as the contemplated animal. The SPs provided herein are from any source, animal, plant, prokaryotic and fungal. Most MTSP20s are of animal origin, including mammalian origin.

As used herein, genetic therapy involves the transfer of heterologous nucleic acid, such as DNA, into certain cells, target cells, of a mammal, particularly a human, with a disorder or conditions for which such therapy is sought. The nucleic acid, such as DNA, is introduced into the selected target cells in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy can also be used to deliver nucleic acid encoding a gene product that replaces a defective gene or supplements a gene product produced by the mammal or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy can also involve delivery of an inhibitor or repressor or other modulator of gene expression.

As used herein, heterologous nucleic acid is nucleic acid that are not normally produced in vivo by the cell in which it is expressed or that mediates or encodes mediators that alter expression of endogenous nucleic acid, such as DNA, by affecting transcription, translation, or other regulatable biochemical processes. Heterologous nucleic acid, such as DNA, can also be referred to as foreign nucleic acid, such as DNA. Any nucleic acid, such as DNA, that one of skill in the art would recognize or consider as heterologous or foreign to the cell in which the nucleic acid is expressed is herein encompassed by heterologous nucleic acid; heterologous nucleic acid includes exogenously added nucleic acid that is also expressed endogenously. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and nucleic acid, such as DNA, that encodes other types of proteins, such as antibodies. Antibodies that are encoded by heterologous nucleic acid can be secreted or expressed on the surface of the cell in which the heterologous nucleic acid has been introduced. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is now expressed.

As used herein, a therapeutically effective product for gene therapy is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease. Also included are biologically active nucleic acid molecules, such as RNAi and antisense.

As used herein, recitation that a polypeptide consists essentially of a protease domain means that the only SP portion of the polypeptide is a protease domain or a catalytically active portion thereof. The polypeptide can optionally, and generally will, include additional non-SP-derived sequences of amino acids.

As used herein, cancer or tumor treatment or agent refers to any therapeutic regimen and/or compound that, when used alone or in combination with other treatments or compounds, can alleviate, reduce, ameliorate, prevent, or place or maintain in a state of remission of clinical symptoms or diagnostic markers associated with deficient angiogenesis.

As used herein, domain refers to a portion of a molecule, e.g., a protein or the encoding nucleic acid, that is structurally and/or functionally distinct from other portions of the molecule.

As used herein, protease refers to an enzyme catalyzing hydrolysis of proteins and/or peptides. It includes zymogen forms and activated forms thereof. For clarity, reference to protease refers to all forms, and particular forms will be specifically designated. For purposes herein, a protease domain includes single and multi-chain forms of a protease domain of an SP protein. For MTSP20, a protease domain also includes single and two chain forms and multi-chain forms of a protease domain.

As used herein, nucleic acids include DNA, RNA and analogs thereof, including peptide nucleic acids (PNA) and mixtures thereof. Nucleic acids can be single or double-stranded. When referring to probes or primers, optionally labeled, with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous of sequence complementary to or identical a gene of interest. Probes and primers can be 10, 20, 30, 50, 100 or more nucleic acids long.

As used herein, nucleic acid encoding a fragment or portion of an SP refers to a nucleic acid encoding only the recited fragment or portion of SP, and not the other contiguous portions of the SP.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. Thus, operatively linked or operationally associated refers to the functional relationship of nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it can be necessary to remove, add or alter 5′ untranslated portions of the clones to eliminate extra, potential inappropriate alternative translation initiation (i.e., start) codons or other sequences that can interfere with or reduce expression, either at the level of transcription or translation. Alternatively, consensus ribosome binding sites (see, e.g., Kozak J. Biol. Chem. 266:19867-19870 (1991)) can be inserted immediately 5′ of the start codon and can enhance expression. The desirability of (or need for) such modification can be empirically determined.

As used herein, a sequence complementary to at least a portion of an RNA, with reference to antisense oligonucleotides, means a sequence having sufficient complementarily to be able to hybridize with the RNA, generally under moderate or high stringency conditions, forming a stable duplex; in the case of double-stranded SP antisense nucleic acids, a single strand of the duplex DNA (or dsRNA) can thus be tested, or triplex formation can be assayed. The ability to hybridize depends on the degree of complementarily and the length of the antisense nucleic acid. Generally, the longer the hybridizing nucleic acid, the more base mismatches with a SP encoding RNA it can contain and still form a stable duplex (or triplex, as the case can be). One skilled in the art can ascertain a tolerable degree of mismatch by use of standard procedures to determine the melting point of the hybridized complex.

For purposes herein, amino acid substitutions can be made in any SPs and protease domains thereof provided that the resulting protein exhibits protease activity. Amino acid substitutions contemplated include conservative substitutions, such as those set forth in Table 1, which do not eliminate proteolytic activity. As described herein, substitutions that alter properties of the proteins, such as removal of cleavage sites and other such sites are also contemplated; such substitutions are generally non-conservative, but can be readily effected by those of skill in the art.

Suitable conservative substitutions of amino acids are known to those of skill in this art and can be made generally without altering the biological activity, for example enzymatic activity, of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224). Also included within the definition, is the catalytically active fragment of an SP, particularly a single chain protease portion. Conservative amino acid substitutions are made, for example, in accordance with those set forth in TABLE 1 as follows:

TABLE 1

| Original residue | Conservative substitution |
|---|---|
| Ala (A) | Gly; Ser, Abu |
| Arg (R) | Lys, orn |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val; Met; Nle; Nva |
| Leu (L) | Ile; Val; Met; Nle; Nv |
| Lys (K) | Arg; Gln; Glu |
| Met (M) | Leu; Tyr; Ile; NLe Val |
| Ornithine | Lys; Arg |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu; Met; Nle; Nv |

Other substitutions are also permissible and can be determined empirically or in accord with known conservative substitutions.

As used herein, Abu is 2-aminobutyric acid; Orn is ornithine.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, a probe or primer based on a nucleotide sequence disclosed herein, includes at least 10, 14, typically at least 16 contiguous sequence of nucleotides of SEQ ID No. 5 or SEQ ID No. 15, and probes of at least 30, 50 or 100 contiguous sequence of nucleotides of SEQ ID No. 5 or SEQ ID No. 15. The length of the probe or primer for unique hybridization is a function of the complexity of the genome of interest.

As used herein, amelioration of the symptoms of a particular disorder by administration of a particular pharmaceutical composition refers to any lessening, whether permanent or temporary, lasting or transient, that can be attributed to or associated with administration of the composition.

As used herein, antisense polynucleotides refer to synthetic sequences of nucleotide bases complementary to mRNA or the sense strand of double-stranded DNA. Admixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double-stranded DNA, inhibition of RNA synthesis (transcription) occurs. The resulting inhibition of translation and/or transcription leads to an inhibition of the synthesis of the protein encoded by the sense strand. Antisense nucleic acid molecules typically contain a sufficient number of nucleotides to specifically bind to a target nucleic acid, generally at least 5 contiguous nucleotides, often at least 14 or 16 or 30 contiguous nucleotides or modified nucleotides complementary to the coding portion of a nucleic acid molecule that encodes a gene of interest, for example, nucleic acid encoding a single chain protease domain of an SP.

As used herein, an array refers to a collection of elements, such as antibodies, containing three or more members. An addressable array is one in which the members of the array are identifiable, typically by position on a solid phase support. Hence, in general the members of the array are immobilized on discrete identifiable loci on the surface of a solid phase.

As used herein, computational docking refers to techniques where molecules, for example, a ligand and receptor or active site, are fitted together based on complementary interactions, for example, steric, hydrophobic or electrostatic interactions. In a typical computational docking protocol, the active site, or sites deemed important for protein activity, of a protein model are defined. A molecular database, such as the Available Chemicals Directory (ACD) or any database of molecules, is screened for molecules that complement the protein model. In computational docking studies, drugs or drug candidates are fitted to the structural variant models based on complementary interactions (e.g., steric, hydrophobic, or electrostatic interactions). Methods for performing such studies are well known and software tools for performing the calculations are widely available (M. Lambert, “Docking Conformationally Flexible Molecules into Protein Binding Sites” in Practical Application of Computer-Aided Drug Design, Charifson, Ed., Marcel Dekker, NY, pp. 243-303; Kurtz (1992) Science 257:1078-1082; Kuntz et al. (1982) J. Mol. Biol. 161:269-288; Stewart et al. (1992) Med. Chem. Res. 1:439-443; Shoichet et al. (1993) Science 259:1445-1450; Shoichet et al. (1991) J. Mol. Biol. 221:327-346).

As used herein, antibody refers to an immunoglobulin, whether natural or partially or wholly synthetically produced, including any derivative thereof that retains the specific binding ability the antibody. Hence antibody includes any protein having a binding domain that is homologous or substantially homologous to an immunoglobulin binding domain. Antibodies include members of any immunoglobulin claims, including IgG, IgM, IgA, IgD and IgE.

As used herein, antibody fragment refers to any derivative of an antibody that is less than full-length, retaining at least a portion of the full-length antibody's specific binding ability. Examples of antibody fragments include, but are not limited to, Fab, Fab′, F(ab)₂, single-chain Fvs (scFV), FV, dsFV diabody and Fd fragments. The fragment can include multiple chains linked together, such as by disulfide bridges. An antibody fragment generally contains at least about 50 amino acids and typically at least 200 amino acids.

As used herein, an Fv antibody fragment is composed of one variable heavy domain (V_{H}) and one variable light domain linked by noncovalent interactions.

As used herein, a dsFV refers to an Fv with an engineered intermolecular disulfide bond, which stabilizes the V_{H}-V_{L} pair.

As used herein, an F(ab)₂ fragment is an antibody fragment that results from digestion of an immunoglobulin with pepsin at pH 4.0-4.5; it can be recombinantly expressed to produce the equivalent fragment.

As used herein, Fab fragments are antibody fragments that result from digestion of an immunoglobulin with papain; they can be recombinantly expressed to produce the equivalent fragment.

As used herein, scFVs refer to antibody fragments that contain a variable light chain (V_{L}) and variable heavy chain (V_{H}) covalently connected by a polypeptide linker in any order. The linker is of a length such that the two variable domains are bridged without substantial interference. Included linkers are (Gly-Ser)ₙ residues with some Glu or Lys residues dispersed throughout to increase solubility.

As used herein, humanized antibodies refer to antibodies that are modified to include human sequences of amino acids so that administration to a human does not provoke an immune response. Methods for preparation of such antibodies are known. For example, to produce such antibodies, the encoding nucleic acid in the hybridoma or other prokaryotic or eukaryotic cell, such as an E. coli or a CHO cell, that expresses the monoclonal antibody is altered by recombinant nucleic acid techniques to express an antibody in which the amino acid composition of the non-variable region is based on human antibodies. Computer programs have been designed to identify such non-variable regions.

As used herein, diabodies are dimeric scFV; diabodies typically have shorter peptide linkers than scFvs, and they generally dimerize.

As used herein, production by recombinant means by using recombinant DNA methods means the use of the well known methods of molecular biology for expressing proteins encoded by cloned DNA.

As used herein the term assessing is intended to include quantitative and qualitative determination in the sense of obtaining an absolute value for an activity of an MTSP, or a domain thereof, present in the sample, and also of obtaining an index, ratio, percentage, visual or other value indicative of the level of the activity. Assessment can be direct or indirect and the chemical species actually detected need not of course be the proteolysis product itself but can for example be a derivative thereof or some further substance.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein the biological activity of a luciferase is its oxygenase activity whereby, upon oxidation of a substrate, light is produced.

As used herein, functional activity refers to a polypeptide or portion thereof that displays one or more activities associated with a full-length protein. Functional activities include, but are not limited to, biological activity, catalytic or enzymatic activity, antigenicity (ability to bind to or compete with a polypeptide for binding to an anti-polypeptide antibody), immunogenicity, ability to form multimers, the ability to specifically bind to a receptor or ligand for the polypeptide.

As used herein, a conjugate refers to the compounds provided herein that include one or more SPs, including an MTSP20, particularly single chain protease domains thereof, and one or more targeting agents. These conjugates include those produced by recombinant means as fusion proteins, those produced by chemical means, such as by chemical coupling, through, for example, coupling to sulfhydryl groups, and those produced by any other method whereby at least one SP, or a domain thereof, is linked, directly or indirectly via linker(s) to a targeting agent.

As used herein, a targeting agent is any moiety, such as a protein or effective portion thereof, that provides specific binding of the conjugate to a cell surface receptor, which, can internalize the conjugate or SP portion thereof. A targeting agent can also be one that promotes or facilitates, for example, affinity isolation or purification of the conjugate; attachment of the conjugate to a surface; or detection of the conjugate or complexes containing the conjugate.

As used herein, an antibody conjugate refers to a conjugate in which the targeting agent is an antibody.

As used herein, derivative or analog of a molecule refers to a portion derived from or a modified version of the molecule.

As used herein, an effective amount of a compound for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

As used herein, equivalent, when referring to two sequences of nucleic acids means that the two sequences in question encode the same sequence of amino acids or equivalent proteins. When equivalent is used in referring to two proteins or peptides, it means that the two proteins or peptides have substantially the same amino acid sequence with only amino acid substitutions (such as, but not limited to, conservative changes such as those set forth in Table 1 above) that do not substantially alter an activity or function of the protein or peptide. When equivalent refers to a property, the property does not need to be present to the same extent (e.g., two peptides can exhibit different rates of the same type of enzymatic activity), but the activities are usually substantially the same. Complementary, when referring to two nucleotide sequences, means that the two sequences of nucleotides are capable of hybridizing, typically with less than 25%, 15%, 5% or 0% mismatches between opposed nucleotides. If necessary the percentage of complementarity will be specified. Typically the two molecules are selected such that they will hybridize under conditions of high stringency.

As used herein, an agent that modulates an activity of a polypeptide, protein or expression of a gene or nucleic acid, either decreases or increases or otherwise alters the activity of the polypeptide or protein or, in some manner up- or down-regulates or otherwise alters expression of the nucleic acid in a cell.

As used herein, inhibitor of an activity of an MTSP encompasses any substance that prohibits or decrease production, post-translational modification(s), maturation, or membrane localization of the SP or any substance that interferes with or decreases the proteolytic efficacy of thereof, particularly of a single chain form in an in vitro screening assay.

As used herein, a method for treating or preventing neoplastic disease means that any of the symptoms, such as the tumor, metastasis thereof, the vascularization of the tumors or other parameters by which the disease is characterized are reduced, ameliorated, prevented, placed in a state of remission, or maintained in a state of remission. It also means that the hallmarks of neoplastic disease and metastasis can be eliminated, reduced or prevented by the treatment. Non-limiting examples of the hallmarks include uncontrolled degradation of the basement membrane and proximal extracellular matrix, migration, division, and organization of the endothelial cells into new functioning capillaries, and the persistence of such functioning capillaries.

As used herein, pharmaceutically acceptable salts, esters or other derivatives of the conjugates include any salts, esters or derivatives that can be readily prepared by those of skill in this art using known methods for such derivatization and that produce compounds that can be administered to animals or humans without substantial toxic effects and that either are pharmaceutically active or are prodrugs.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound is regenerated by metabolic processes. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, a drug identified by the screening methods provided herein refers to any compound that is a candidate for use as a therapeutic or as a lead compound for the design of a therapeutic. Such compounds can be small molecules, including small organic molecules, peptides, peptide mimetics, antisense molecules or dsRNA, such as RNAi, antibodies, fragments of antibodies, recombinant antibodies and other such compounds that can serve as drug candidates or lead compounds.

As used herein, a peptidomimetic is a compound that mimics the conformation and certain stereochemical features of the biologically active form of a particular peptide. In general, peptidomimetics are designed to mimic certain desirable properties of a compound, but not the undesirable properties, such as flexibility, that lead to a loss of a biologically active conformation and bond breakdown. Peptidomimetics may be prepared from biologically active compounds by replacing certain groups or bonds that contribute to the undesirable properties with bioisosteres. Bioisosteres are known to those of skill in the art. For example the methylene bioisostere CH₂S has been used as an amide replacement in enkephalin analogs (see, e.g., Spatola (1983) pp. 267-357 in Chemistry and Biochemistry of Amino Acids, Peptides, and Proteins, Weistein, Ed. volume 7, Marcel Dekker, New York). Morphine, which can be administered orally, is a compound that is a peptidomimetic of the peptide endorphin. For purposes herein, cyclic peptides are included among peptidomimetics.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences can be cis acting or can be responsive to trans acting factors. Promoters, depending upon the nature of the regulation, can be constitutive or regulated. Exemplary promoters contemplated for use in prokaryotes include the bacteriophage T7 and T3 promoters.

As used herein, a receptor refers to a molecule that has an affinity for a given ligand. Receptors can be naturally-occurring or synthetic molecules. Receptors can also be referred to in the art as anti-ligands. As used herein, the receptor and anti-ligand are interchangeable. Receptors can be used in their unaltered state or as aggregates with other species. Receptors can be attached, covalently or noncovalently, or in physical contact with, a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants (such as on viruses, cells, or other materials), drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic (ligand) selection;

b) antibodies: identification of a ligand-binding site on the antibody molecule that combines with the epitope of an antigen of interest can be investigated; determination of a sequence that mimics an antigenic epitope can lead to the development of vaccines of which the immunogen is based on one or more of such sequences or lead to the development of related diagnostic agents or compounds useful in therapeutic treatments such as for auto-immune diseases

c) nucleic acids: identification of ligand, such as protein or RNA, binding sites;

d) catalytic polypeptides: polymers, including polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant (see, e.g., U.S. Pat. No. 5,215,899);

e) hormone receptors: determination of the ligands that bind with high affinity to a receptor is useful in the development of hormone replacement therapies; for example, identification of ligands that bind to such receptors can lead to the development of drugs to control blood pressure; and

f) opiate receptors: determination of ligands that bind to the opiate receptors in the brain is useful in the development of less-addictive replacements for morphine and related drugs.

As used herein, sample refers to anything that contains an analyte for which an analyte assay is desired. The sample can be a biological sample, such as a biological fluid or a biological tissue. Examples of biological fluids include urine, blood, plasma, serum, saliva, semen, stool, sputum, cerebral spinal fluid, tears, mucus, sperm, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

Those of skill in this art know that the washing step selects for stable hybrids and also know the ingredients of SSPE (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phosphate-buffered 0.18 NaCl. Further, those of skill in the art recognize that the stability of hybrids is determined by Tₘ, which is a function of the sodium ion concentration and temperature (Tₘ=81.5° C.−16.6(log₁₀[Na⁺])+0.41 ((% G+C)−600/1)), so that the only parameters in the wash conditions critical to hybrid stability are sodium ion concentration in the SSPE (or SSC) and temperature.

It is understood that equivalent stringencies can be achieved using alternative buffers, salts and temperatures. By way of example and not limitation, procedures using conditions of low stringency are as follows (see also Shilo and Weinberg, Proc. Natl. Acad. Sci. USA 78:6789-6792 (1981)): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and reexposed to film. Other conditions of low stringency which can be used are well known in the art (e.g., as employed for cross-species hybridizations).

Procedures using conditions of moderate stringency include, for example, but are not limited to, procedures using such conditions of moderate stringency, are as follows: Filters containing DNA are pretreated for 6 hours at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Other conditions of moderate stringency which can be used are well-known in the art. Washing of filters is done at 37° C. for 1 hour in a solution containing 2X SSC, 0.1% SDS.

By way of example and not way of limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency which can be used are well known in the art.

The term substantially identical or substantially homologous or similar varies with the context as understood by those skilled in the relevant art and generally means at least 60% or 70%, preferably means at least 80%, 85% or more preferably at least 90%, and most preferably at least 95% identity.

As used herein, substantially identical to a product means sufficiently similar so that the property of interest is sufficiently unchanged so that the substantially identical product can be used in place of the product.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, target cell refers to a cell that expresses an MTSP in vivo.

As used herein, test substance (or test compound) refers to a chemically defined compound (e.g., organic molecules, inorganic molecules, organic/inorganic molecules, proteins, peptides, nucleic acids, oligonucleotides, lipids, polysaccharides, saccharides, or hybrids among these molecules such as glycoproteins, etc.) or mixtures of compounds (e.g., a library of test compounds, natural extracts or culture supernatants, etc.) whose effect on an MPSP, particularly a single chain form that includes a protease domain or a sufficient portion thereof for activity, as determined by an in vitro method, such as the assays provided herein, is tested.

As used herein, the terms a therapeutic agent, therapeutic regimen, radioprotectant or chemotherapeutic mean conventional drugs and drug therapies, including vaccines, which are known to those skilled in the art. Radiotherapeutic agents are well known in the art.

As used herein, treatment means any manner in which the symptoms of a condition, disorder or disease are ameliorated or otherwise beneficially altered. Treatment also encompasses any pharmaceutical use of the compositions herein.

As used herein, vector (or plasmid) refers to discrete elements that are used to introduce heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. An expression vector includes vectors capable of expressing DNA that is operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, protein binding sequence refers to a protein or peptide sequence or a portion of other macromolecules that is capable of specific binding to protein or peptide sequences generally, to a set of protein or peptide sequences or to a particular protein or peptide sequence.

As used herein, epitope tag refers to a short stretch of amino acid residues corresponding to an epitope to facilitate subsequent biochemical and immunological analysis of the epitope tagged protein or peptide. Epitope tagging is achieved by including the sequence of the epitope tag to the protein-encoding sequence in an appropriate expression vector. Epitope tagged proteins can be affinity purified using highly specific antibodies raised against the tags.

As used herein, metal binding sequence refers to a protein or peptide sequence that is capable of specific binding to metal ions generally, to a set of metal ions or to a particular metal ion.

As used herein, a cellular extract refers to a preparation or fraction which is made from a lysed or disrupted cell. A tissue extract is a preparation or fraction which is made from a lysed or disrupted tissue.

As used herein, a combination refers to any association between two or among more items.

As used herein, a composition refers to any mixture. It can be a solution, a suspension, liquid, powder, a paste, aqueous, non-aqueous or any combination thereof.

As used herein, fluid refers to any composition that can flow. Fluids thus encompass compositions that are in the form of semi-solids, pastes, solutions, aqueous mixtures, gels, lotions, creams and other such compositions.

As used herein, an agent is said to be randomly selected when the agent is chosen randomly without considering the specific sequences involved in the association of a protein alone or with its associated substrates, binding partners, etc. An example of randomly selected agents is the use a chemical library or a peptide combinatorial library, or a growth broth of an organism or conditioned medium.

As used herein, an agent is said to be rationally selected or designed when the agent is chosen on a non-random basis which takes into account the sequence of the target site and/or its conformation in connection with the agent's action. Agents can be rationally selected or rationally designed by employing the peptide sequences that make up these sites. For example, a rationally selected peptide agent can be a peptide whose amino acid sequence is identical to the ATP or calmodulin binding sites or domains.

For clarity of disclosure, and not by way of limitation, the detailed description is divided into the subsections that follow.

B. MTSP20 Polypeptides, Muteins, Derivatives and Analogs Thereof

1. MTSPs

The MTSPs are a family of transmembrane serine proteases that are found in mammals and also other species. MTSPs are of interest because they appear to be expressed and/or activated at levels different from those in tumor cells normal cells, or have functional activity that is different in tumor cells from normal cells, such as by an alteration in a substrate therefor, or a cofactor or a receptor.

The MTSPs share a number of common structural features including: a proteolytic extracellular domain; a transmembrane domain, with a hydrophobic domain near the N-terminus; a short cytoplasmic domain; and a variable length stem region that may contain additional modular domains. The proteolytic domains share sequence homology including conserved His, Asp, and Ser residues necessary for catalytic activity that are present in conserved motifs. The MTSPs are normally synthesized as zymogens and can be activated to multi-chain forms by cleavage. A single chain proteolytic domain can function in vitro and, hence is useful in in vitro assays for identifying agents that modulate an activity of members of this family.

For purposes herein, a protease domain of an MTSP20 does not have to result from activation cleavage, which produces a multi-chain activated product, but rather includes single chain polypeptides where the N-termini include the consensus sequence ↓VVGG, ↓IVGG, ↓VGLL, ↓ILGG, ↓IVQG or ↓IVNG ↓IASG or other such motif. Such polypeptides, although not the result of activation cleavage and not multi-chain forms, exhibit proteolytic (catalytic) activity. These protease domain polypeptides are used in assays to screen for agents that modulate an activity of the MTSP20.

The MTSP family is a target for therapeutic intervention and also some members can serve as diagnostic markers for tumor development, growth and/or progression. As discussed, the members of this family are involved in proteolytic processes that are implicated in tumor development, growth and/or progression. This implication is based upon their functions as proteolytic enzymes in processes related to ECM degradation and/or remodeling and activation of pro-growth factors, pro-hormones or pro-angiogenic compounds. In addition, their levels of expression or level of activation or their apparent activity resulting from substrate levels or alterations in substrates and levels thereof differs in tumor cells and non-tumor cells in the same tissue. Similarly the level of co-factors or receptors for these proteases can vary between tumor and non-tumor cells. Hence, protocols and treatments that alter their activity, such as their proteolytic activities and roles in signal transduction, and/or their expression, such as by contacting them with a compound that modulates their activity and/or expression, could impact tumor development, growth and/or progression. Also, in some instances, the level of activation and/or expression can be altered in tumors, such as lung carcinoma, colon adenocarcinoma and ovarian carcinoma.

2. Protease Involvement in Angiogenesis

In the initial stages of angiogenesis, microvascular endothelial cells of preexisting blood vessels locally degrade the underlying basal lamina and invade into the stroma of the tissue to be vascularized. It has been shown that this process requires a wide array of degradative enzymes (Mignatti and Rifkin, Enzyme Protein, 49(1-3):117-37 (1996)). Components of the plasminogen activator (PA)-plasmin system and the matrix metalloproteinase (MMP) family play important roles. PAs trigger a proteinase cascade that results in the generation of high local concentrations of plasmin and active MMPs. This increase in proteolytic activity has three major consequences: it permits extracellular matrix degradation and invasion of the vessel basal lamina, generates extracellular matrix (ECM) degradation products that are chemotactic for endothelial cells, and activates and mobilizes growth factors localized in the ECM. Five proteases, urokinase-type plasminogen activator, factor XII, protein C, trypsinogen IV, and a membrane-type serine protease 1 (MTSP1) that may be involved in these processes have been identified.

At least nine different MMPs have been identified. Among these are gelatinase A, which degrades collagen types IV and V, elastin and laminin and which is frequently overexpressed in stromal cells of malignant tumors (Vassalli and Pepper, Nature, 370:14-5 (1994)). Nucleic acid encoding a matrix metalloproteinase with a potential transmembrane domain has been cloned (Sato et al., Nature, 370:61-5 (1994)). Expression of the cloned gene product on the cell surface induces specific activation of pro-gelatinase A in vitro and enhances cellular invasion of the reconstituted basement membrane. Tumor cells of invasive lung carcinomas, which contain activated forms of gelatinase A, were found to express the transcript and the gene product. Inhibition of protease activity through the use of wild-type and engineered ecotins results in inhibition of rat prostate differentiation and retardation of the growth of human PC-3 prostatic cancer tumors (see, Takeuchi et al. Proc. Natl. Acad. Sci. (USA), 96(20):11054-61 (1999)).

Thus, endothelial cells and proteases play key roles in angiogenesis and related processes. Furthermore, proteases can serve as therapeutic targets and points of intervention in processes relying on protease activities.

As noted above, aberrant angiogenesis and processes related thereto have a role in a variety of disorders, including cancers, diabetic retinopathies, hyperproliferative disorders, restenosis, and others. As provided herein, because proteases are involved either directly or indirectly in angiogenesis, which is aberrant in a variety of disorders, altering an activity of proteases involved in angiogenesis could treat the resulting disorders. Thus, proteases involved in angiogenesis can serve as therapeutic targets and also in drug screening methodologies to identify compounds that modulate, particularly inhibit, angiogenesis.

MTSP20, provided herein is an endotheliase. It is an endothelial cell transmembrane proteins, which in view of the role(s) of endothelial cells and proteases in angiogenesis and related processes, is directly or indirectly involved in the process of angiogenesis or processes related thereto. Thus, MTSP20s are therapeutic, diagnostic and/or prognostic targets for intervention in the process of angiogenesis, either for inhibition or activation thereof. In addition, protease domains (extracellular domains) provide a means to screen for compounds that modulate angiogenesis.

The MTSP20s provided herein and the screening methods provided herein permit discovery of candidate compounds that modulate processes involved in the establishment and maintenance of the vasculature. These methods provide a means to select compounds that selectively bind to the MTSP20s or interact therewith resulting an increase or decrease in a protease activity of the MTSP20. Since MTSP20s occur on endothelial cells, which are intimately involved in processes related to establishment and maintenance of the vasculature, the compounds identified by the methods herein can have activity as anti-angiogenic or pro-angiogenic agents.

3. MTSP20

Thus, MTSP20 is of interest because it is expressed or is active in tumor cells. In addition, as noted MTSP20 is also an endotheliase, and is expressed or is active in endothelial cells, particularly those involved in angiogenesis (angiogenic endothelial cells). The MTSPs provided herein can serve as a diagnostic marker for particular tumors, by virtue of a level of activity and/or expression or function in a subject (i.e. a mammal, particularly a human) with neoplastic disease, compared to a subject or subjects that do not have the neoplastic disease. In addition, detection of activity (and/or expression) in a particular tissue can be indicative of neoplastic disease.

It is shown herein, that MTSP20s provided herein are expressed and/or activated in certain tumors; hence their activation or expression can serve as a diagnostic marker for tumor development, growth and/or progression. In other instances, the MTSP polypeptide can exhibit altered activity by virtue of a change in activity or expression of a co-factor, a substrate or a receptor. In addition, in some instances, these MTSPs and/or variants thereof can be shed from cell surfaces. Detection of the shed MTSPs, particularly the extracellular protease domains, in body fluids, such as serum, blood, saliva, cerebral spinal fluid, synovial fluid and interstitial fluids, urine, sweat and other such fluids and secretions, can serve as a diagnostic tumor marker. In particular, detection of higher levels of such shed polypeptides in a subject compared to a subject known not to have any neoplastic disease or compared to earlier samples from the same subject, can be indicative of neoplastic disease in the subject.

a. Polypeptides and Muteins

Provided herein are isolated substantially pure single chain and multi-chain polypeptides that contain a protease domain of an MTSP20. The polypeptides also can include other non-MTSP sequences of amino acids, but includes a protease domain or a sufficient portion thereof to exhibit catalytic activity in any in vitro assay that assess such protease activity, such as any provided herein.

MTSP20 polypeptides provided herein are expressed or activated by or in tumor cells or endothelial, typically at a level that differs from the level in which they are expressed by or activated in a non-tumor cell of the same type. Hence, for example, if the MTSP is expressed in an cervical tumor cell, it is expressed or active at a different level from the level in non-tumor cervical cells. MTSP20 expression or activation can be indicative of cervical, lung, esophogeal, colon, prostate, uterine, pancreatic, breast and other tumors.

Isolated, substantially pure proteases that include protease domains or a catalytically active portion thereof are provided. Provided are single chain forms and multi-chain forms of the MTSP20. Protease domains can be included in a longer protein, and such longer protein is optionally a MTSP20 zymogen up to a full-length zymogen. Exemplary MTSP20-encoding nucleic acid and protein sequences of a protease domain are set forth in SEQ ID Nos. 5 and 6. Full-length MTSP20-encoding nucleic acid molecules that contain the sequence set forth in SEQ ID No. 5 (with amino acid 247 optionally replaced with 1) or SEQ ID No. 16 and polypeptides that include the sequence of amino acids set forth in SEQ ID No. 6 (with amino acid 247 optionally replaced with 1) or SEQ ID No. 16 or catalytically active portions thereof are also provided herein. Thus, an MTSP20 polypeptide includes the sequence of amino acids set forth in SEQ ID No. 6 or residues 624-642 of SEQ ID No. 16 are provided. Smaller portions thereof that retain protease activity are contemplated.

Substantially purified MTSP20 protease is encoded by a nucleic acid that hybridizes to a nucleic acid molecule that encodes a protease domain (or two) within the sequence of amino acids set forth in SEQ. ID No. 16 under at least moderate, generally high, stringency conditions, such that a protease domain encoding nucleic acid thereof hybridizes along its full-length or at least 70%, 80% or 90% of the full-length. In certain embodiments the substantially purified MTSP protease is a single chain polypeptide that includes substantially the sequence of amino acids set forth in SEQ ID No. 6 or a catalytically active portion thereof.

Also included are substantially purified MTSP20 zymogens, activated two, three and other multi-chain forms, single chain protease domains and two chain protease domains. These polypeptides are encoded by nucleic acids that include sequences encoding a protease domain that exhibits proteolytic activity and that hybridizes to a nucleic acid molecule having a nucleotide sequence set forth in SEQ ID No. 5 or SEQ ID No. 16, typically under moderate, generally under high stringency, conditions and generally along the full-length or along at least about 70%, 80% or 90% of the full-length (or substantially the full-length) of a protease domain. Splice variants are also contemplated herein.

b. Structural Features

MTSP20 has two trypsin-like serine protease domains in tandem, hereafter referred to as MTSP20-PD1 (aa 113 to aa 343) and MTSP20-PD2 (aa 375 to aa 596). Both protease domains are characterized by the presence of a protease activation cleavage site at the beginning of the domain and the catalytic triad residues (histidine, aspartate and serine) in 3 highly-conserved regions of the catalytic domain.

MTSP20-PD1 has the following features: a protease cleavage site ( . . . K₁₁₂↓P₁₁₃QEGN . . . , where ↓ indicates the cleavage site); catalytic triad residues (H₁₅₂, D₂₀₃ and S299); the following cysteine pairings within the protease domain (C₁₃₇-C₁₅₃, C₂₃₃-C₃₀₅, C₂₆₄-C₂₈₄ and C₂₉₅-C₃₂₄); an unpaired cysteine (C₂₁₉) in the protease domain is predicted to pair with C₁₀₃.

MTSP20-PD2 has the following features: the protease cleavage site ( . . . R₃₇₅↓T₃₇₆AGPQ . . . ); catalytic triad residues (H416, D₄₅₇ and S₅₅₃); the following cysteine pairings within the protease domain (C₄₀₁-C₄₁₇, C₅₁₉-C₅₃₉ and C₅₄₉-C₅₇₇); the unpaired cysteine (C₄₇₇) in the protease domain is predicted to pair with C₃₇₁.

c. Protease Domains

MTSP protease domains include the single chain protease domains and multi-chain domains of MTSP20. In the exemplary MTSP20, there are two protease domains: residues 113-343 (PD1) of SEQ ID No. 16 (see, also SEQ ID No. 6) and residues 376-642 (PD2) of SEQ ID No. 16. Also contemplated are catalytically active portions thereof, forms that include both domains and multi-chain forms thereof. Each of these can be a single chain form or in a two chain form formed by cleavage of the activation cleavage site after binding of an unpaired Cys in the protease domain (i.e. Cys₂₁₉ in PD1 and Cys₄₇₇ in PD2) paired with a Cys outside the domain C₁₀₃ for PD1 and C₃₇₁ for PD2 to produce a two chain or multi-chain polypeptide that includes the protease domain plus at least the residues that are between the linked cysteines prior to cleavage and after cleavage.

A protease domain or a plurality thereof can be employed as substantially isolated proteins, or as part of a full-length MTSP20, or can be a part of a larger polypeptide that includes other non-MTSP sequences of amino acids, but includes a protease domain or a plurality of domains or a sufficient portion thereof to exhibit catalytic activity in any in vitro assay that assess such protease activity, such as any provided herein. Also provided are multi-chain activated forms of the full length MTSP20 protease and also multi-chain forms of the protease domains. Thus, isolated, substantially pure proteases that include the protease domains (one or two protease domains) or catalytically active portions thereof as single chain forms of SPs are provided. The protease domains can be included in a longer protein, and such longer protein is optionally the activated MTSP20 protein, up to and including a full-length, or an MTSP20 zymogen.

In particular, exemplary protease domains include at least a sufficient portion of sequences of amino acids set forth of SEQ ID No. 6 or SEQ ID No. 16 (encoded by nucleotides in SEQ ID No. 5 or SEQ ID No. 15) to retain catalytic activity in vitro.

As noted, protease domains of an MTSP are single-chain polypeptides or multi-chain polypeptides, with an N-terminus (such as IV, VV, IL and II or other N-terminus) generated at the cleavage site (generally having the consensus sequence R↓VVGG, R↓IVGG, R↓IIVQ, R↓IVNG, R↓ILGG, R↓VGLL, R↓ILGG or a variation thereof or a site at an homologous locus; an N-terminus R↓V or R↓I, where the arrow represents the cleavage point) when a zymogen is activated. A protease domain of an exemplary MTSP20 is produced by activation cleavage between reside 112 and 113 and includes the sequence K₁₁₂↓P₁₁₃QEGN16 (K↓P) as set forth in SEQ ID No. 16. A second activation cleavage site occurs between residues 375 and 376 and includes the R₃₇₅↓T₃₇₆AGPQ of sequence ID No. 16. The resulting protease domains span resudes 113 to 343 of SEQ ID No. 16 and residues 376-642 of SEQ ID No. 16; see also SEQ ID No. 6, where residue 247 (1622 of SEQ ID No. 16) is M. A single chain form includes the sequence of amino acids set forth in SEQ ID No. 6 or as residues 1-343 or 113-343 of SEQ ID No. 16 and catalytically active fragments thereof are provided. Thus provided are full-length MTSPs, which include at least a residue 624 (as set forth in SEQ ID No. 16), particularly residues 624 and one or more contiguous residues up to 642 (of SEQ ID No. 16 or equivalent amino acids) are provided (i.e., a splice variant that includes such residue(s)).

Also provided are single chain protease domains that include residues 1-343 or 113-343 of SEQ ID No. 16 or SEQ ID No. 6 and/or catalytically active fragments thereof or variants thereof (as described herein), are contemplated herein. Multiple chain, particularly two and three chain forms, are provided. Also provided are polypeptides that are MTSP20 polypeptides as described that have 60%, 70%, 80%, 90%, or 95% sequence identity there with and/or is:

a) a polypeptide that includes the sequence of amino acids set forth in SEQ ID No. 6, where residue 247 is M or 1, as the only MTSP20 sequence in the polypeptide;

b) a polypeptide that includes the sequence of amino acids set forth in SEQ ID No. 16 or a catalytically active portion thereof;

c) a polypeptide that includes residue 624 or a contiguous sequence of amino acids that includes residue 624 and one or more contingious residues through residue 642 as set forth in SEQ ID No. 16;

d) a polypeptide encoded by a sequence of nucleotides that hybridizes under conditions of low, moderate or high stringency to the sequence of nucleotides set forth in SEQ ID No. 5 or to SEQ ID No. 15 or hybridizes along at least 70%, 80%, 90% or 95% of its full-length that encodes any of the polypeptides of a), b) or c)

e) a polypeptide that includes the sequence of amino acids set forth in SEQ ID No. 6 or SEQ ID No. 16 or a catalytically active portion thereof;

f) a polypeptide that includes a sequence of amino acids having at least about 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence of amino acids set forth in SEQ ID No. 6 or SEQ ID No. 16; and/or

g) a protease domain of a polypeptide encoded by a splice variant of a sequence of nucleotides that encodes an MTSP20 of any of a)-f); and

h) for use in the screening and diagnostic methods MTSP20 polypeptides also include those that include the sequence of amino acids set forth in SEQ ID No. 17 or a protease domain thereof and those that are encoded by a sequence of nucleotides that hybridizes under conditions of low, moderate or high stringency to the sequence of nucleotides set forth in SEQ ID No. 17 along at least 70%, 80%, 90%, 95% or 100% of its full-length to the full-length polypeptide or protease domain thereof.

Also provided are polypeptides that are encoded by nucleic acid molecules that meet criteria specified below as (a)-(f) in the section designated “Nucleic Acids.”

d. Muteins and Derivatives

Full-length MTSP20, zymogen and activated forms thereof and MTSP20 protease domains, portions thereof, and muteins and derivatives of such polypeptides are provided. The domains, fragments, derivatives or analogs of an MTSP20 that are functionally active are capable of exhibiting one or more functional activities associated with the MTSP20 polypeptide, such as serine protease activity, immunogenicity and antigenicity, are provided.

Among the derivatives are those based on animal MTSP20s, including, but are not limited to, rodent, such as mouse and rat; fowl, such as chicken; ruminants, such as goats, cows, deer, and sheep; ovine, such as pigs; and humans. For example, MTSP20 derivatives can be made by altering their sequences by substitutions, additions or deletions. MTSP20 derivatives include, but are not limited to, those containing, as a primary amino acid sequence, all or part of the amino acid sequence of MTSP20, including altered sequences in which functionally equivalent amino acid residues are substituted for residues within the sequence resulting in a silent change. For example, one or more amino acid residues within the sequence can be substituted by another amino acid of a similar polarity which acts as a functional equivalent. Substitutes for an amino acid within the sequence can be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid (see, e.g., Table 1). Muteins of the MTSP20 or a domain thereof, such as a protease domain, in which up to about 10%, 20%, 30%, 40%, 50%, 60%, 70%, 80%, 85%, 90% or 95% of the amino acids are replaced with another amino acid are provided. Generally such muteins retain at least about 1%, 2%, 3%, 5%, 7%, 8%, 10%, 20%, 30%, 40%, 50%, 60%, 70%, 80% or 90% of a protease activity of the unmutated protein. Those of skill in the art recognize that a polypeptide that retains at least 1% of an activity of the wild-type protease is sufficiently active for use in screening assays or for other applications.

Included among the polypeptides provided herein are the MTSP20 protease domain or a polypeptide with amino acid changes such that the specificity and protease activity remains substantially unchanged or changed (increased or decreased) by a specified percentage, such as 10%, 20%, 30%, 40%, 50%, 60%, 70%, 80%, 85%, 90%, 91%, 92%, 93%, 94%, 95%, 96%, 97%, 98%, 99%, 99.5%. In particular, a substantially purified mammalian MTSP polypeptide is provided that has a transmembrane (TM) domain two protease catalytic domains.

Also provided is a substantially purified protein containing a sequence of amino acids that has at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to the MTSP20 where the percentage identity is determined using standard algorithms and gap penalties that maximize the percentage identity. The human MTSP20 polypeptide is included, although other mammalian MTSP20 polypeptides are contemplated. The precise percentage of identity can be specified if needed.

Muteins in which one or more of the Cys residues, particularly, a residue that is paired in the activated two form, but unpaired in a protease domain alone is/are replaced with any amino acid, typically, although not necessarily, a conservative amino acid residue, such as Ser, are contemplated. Muteins of MTSP20, particularly those in which Cys residues, such as the Cys₂₁₉ and Cys₄₇₇ in the single chain protease domains, is replaced with another amino acid, such as Ser, Gly or Ala, that does not eliminate the activity, are provided.

Also provided are substantially purified MTSP20 polypeptides and functional domains thereof, including catalytically active domains and portions, that have at least about 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with a protease domain that includes a sequence of amino acids set forth in SEQ ID No. 6 or catalytically active fragments thereof.

Muteins of the protein are also provided in which amino acids are replaced with other amino acids. Among the muteins are those in which the Cys residues, is/are replaced typically with a conservative amino acid residues, such as a serine. Such muteins are also provided herein. Muteins are provided in which 10%, 20%, 30%, 35%, 40%, 45%, 50% or more of the amino acids are replaced but the resulting polypeptide retains at least about 1%, 2%, 3,%, 5%, 7%, 8%, 10%, 20%, 30%, 35%, 40%, 45%, 50%, 60%, 70%, 80%, 90% or 95% of the catalytic activity as the unmodified form for the same substrate.

Muteins can be made by making conservative amino acid substitutions and also non-conservative amino acid substitutions. For example, amino acid substitutions that desirably alter properties of the proteins can be made. In one embodiment, mutations that prevent degradation of the polypeptide can be made. Many proteases cleave after basic residues, such as R and K; to eliminate such cleavage, the basic residue is replaced with a non-basic residue. Also, non-conservative changes at amino acids outside of a protease domain can be effected without altering protease activity. Non-conservative changes at amino acids that are responsible for activities other than protease activity may be desirable. For example, interaction of a protease with an inhibitor can be blocked while retaining catalytic activity by effecting a non-conservative change at the site of interaction of the inhibitor with the protease. Similarly, receptor binding can be altered without altering catalytic activity by effecting a non-conservative or conservative change at a site of interaction of the receptor with the protease.

Antigenic epitopes that contain at least 4, 5, 6, 7, 8, 9, 10, 11, 12, 13, 14, 15, 20, 25, 30, 40, 50, and typically 10-15 amino acids of the MTSP20 polypeptide are provided. These antigenic epitopes are used, for example, to raise antibodies. Antibodies specific for each epitope or combinations thereof and for single-, two- and other multi-chain forms are also provided. Antibodies that bind with higher affinity to an MTSP that includes amino acid 624-642 (or includes two protease domains) than to an MTSP20, such as the polypeptide of SEQ ID No. 17, that lacks such residues are provided.

4. Nucleic Acid Molecules, Vectors and Plasmids, Cells and Expression of MTSP20 Polypeptides

a. Nucleic Acid Molecules

Due to the degeneracy of nucleotide coding sequences, other nucleic sequences which encode substantially the same amino acid sequence as a MTSP are contemplated. These include but are not limited to nucleic acid molecules that include all or portions of MTSP20-encoding genes that are altered by the substitution of different codons that encode the amino acid residue within the sequence, thus producing a silent change.

1. Nucleic Acids

Also provided herein are nucleic acid molecules that encode MTSP20 polypeptides and the encoded proteins. In particular, nucleic acid molecules encoding MTSP20 from animals, including splice variants thereof are provided. The encoded proteins are also provided. Also provided are functional domains thereof. For each of the nucleic acid molecules provided, the nucleic acid can be DNA or RNA or PNA or other nucleic acid analogs or can include non-natural nucleotide bases. Also provided are isolated nucleic acid molecules that include a sequence of nucleotides complementary to the nucleotide sequence encoding an MTSP.

Also provided are nucleic acid molecules that encode single chain or multi-chain MTSP proteases that have proteolytic activity in an in vitro proteolysis assay and that have at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with the full-length of a protease domain of an MTSP20 polypeptide, or that hybridize along their full-length or along at least about 70%, 80% or 90% of the full-length nucleic acid to a nucleic acids that encode a protease domain, particularly under conditions of moderate, generally high, stringency. As above, the encoded polypeptides contain the protease as a single chain; activated forms thereof can be produced and are provided.

In one embodiment, a nucleic acid molecule that encodes an MTSP, designated MTSP20 is provided. The nucleic acid molecule includes the open reading frame in the sequence of nucleotides set forth in SEQ ID No. 5 or SEQ ID No. 15. Also provided are nucleic acid molecules that hybridize under conditions of at least low stringency, moderate stringency, and generally high stringency to the following sequence of nucleic acids (SEQ ID No. 5) particularly to the open reading frame encompassed by nucleotides that encode a single protease domain thereof, or any domain of MTSP20.

In certain embodiments, the isolated nucleic acid fragment hybridizes to the nucleic acid having the nucleotide sequence set forth in SEQ ID No. 5 under high stringency conditions, and generally contains the sequence of nucleotides set forth as nucleotides 1-690 in SEQ ID No. 5. The protein contains a transmembrane domain (TM) and serine protease domains and can contain additional domains.

Also provided, are muteins of the nucleic acid molecules that encode polypeptides in which amino acids are replaced with other amino acids. Among the muteins are those in which the Cys residue-encoding codons, is/are replaced with other amino acid residues, such as a codon encoding a serine. Such muteins are also provided herein. Each of such domains is provided herein as are nucleic acid molecules that include sequences of nucleotides encoding such domains. Some MTSPs can additionally include a LDLR domain, a scavenger-receptor cysteine rich (SRCR) domain, CUB domain and other domains.

The isolated nucleic acid fragment is DNA, including genomic or cDNA, or is RNA, or can include other components, such as peptide nucleic acid (PNA) and other nucleotide analogs. The isolated nucleic acid can include additional components, such as heterologous or native promoters, and other transcriptional and translational regulatory sequences, these genes can be linked to other genes, such as reporter genes or other indicator genes or genes that encode indicators.

Also provided are nucleic acid molecules that hybridize to the above-noted sequences of nucleotides encoding MTSP20 at least at low stringency, moderate stringency, and typically at high stringency, and that encode a protease domain and/or a plurality of protease domains and/or the full-length protein or at least 60%, 70%, 80% or 90% of the full-length protease domain or other domains of an MTSP20 or a splice variant or allelic variant thereof. Generally the molecules hybridize under such conditions along their full-length or along at least 70%, 80% or 90% of the full-length for at least one domain and encode at least one domain, such as a protease domain or an extracellular domain, of the polypeptide. In particular, such nucleic acid molecules include any isolated nucleic acid fragment that encodes at least one domain of a membrane serine protease, that (1) contains a sequence of nucleotides that encodes the protease or a domain thereof, and (2) is selected from among:

(a) a sequence of nucleotides that encodes the MTSP20 polypeptide or a domain thereof that includes a sequence of nucleotides set forth in SEQ ID No. 5, particularly nucleotides 1-690, or SEQ ID No. 15;

(b) a sequence of nucleotides that encodes such portion or the full-length protease and hybridizes under conditions of high stringency, generally to nucleic acid that is complementary to a mRNA transcript present in a mammalian cell that encodes such protein or fragment thereof;

(c) a sequence of nucleotides that encodes a protease or a domain thereof that includes a sequence of nucleotides having at least about 60%, 70%, 80%, 90% or 95% sequence identity with the sequence set forth in SEQ ID No. 5, particularly nucleotides 360-1052 or 1148-1952 in SEQ ID No. 15;

(d) a sequence of nucleotides that encodes a transmembrane protease or domain thereof that includes a sequence of amino acids encoded by such portion or the full-length open reading frame;

(e) a sequence of nucleotides that encodes the MTSP20 polypeptide or a domain thereof that includes a sequence of nucleotides set having at least about 60%, 70%, 80%, 90% or 95% sequence identity with the sequence set forth in SEQ ID No. 5 or SEQ ID No. 15; and

(f) a sequence of nucleotides that encodes the MTSP20 polypeptide that includes a sequence of amino acids encoded by a sequence of nucleotides that encodes such subunit and hybridizes under conditions of low, moderate or high stringency to DNA that is complementary to the mRNA transcript.

The isolated nucleic acids can contain least 10 nucleotides, 25 nucleotides, 50 nucleotides, 100 nucleotides, 150 nucleotides, or 200 nucleotides or more contiguous nucleotides of an MTSP20-encoding sequence, or a full-length SP coding sequence. In another embodiment, the nucleic acids are smaller than 35, 200 or 500 nucleotides in length. Nucleic acids that hybridize to or are complementary to an MTSP20-encoding nucleic acid molecule can be single or double-stranded. For example, nucleic acids are provided that include a sequence complementary to (specifically are the inverse complement of) at least 10, 25, 50, 100, or 200 nucleotides or the entire coding region of an MTSP20 encoding nucleic acid, particularly a protease domain thereof. For MTSP20 the full-length protein or a domain or domains or active fragment(s) thereof is(are) also provided.

2. Probes, Primers, Antisense Oligonucleotides and dsRNA

Also provided are fragments thereof that can be used as probes or primers and that contain at least about 10 nucleotides, 14 nucleotides, generally at least about 16 nucleotides, often at least about 30 nucleotides. The length of the probe or primer is a function of the size of the genome probed; the larger the genome, the longer the probe or primer required for specific hybridization to a single site. Those of skill in the art can select appropriately sized probes and primers. Generally probes and primers as described are single-stranded. Double stranded probes and primers can be used, if they are denatured when used.

Probes and primers derived from the nucleic acid molecules are provided. Such probes and primers contain at least 8, 14, 16, 30, 100 or more contiguous nucleotides with identity to contiguous nucleotides of an MTSP20, and probes of at least 14, 16, 30, 50 or 100 contiguous sequence of nucleotides of SEQ ID No. 5 or SEQ ID No. 15. The probes and primers are optionally labelled with a detectable label, such as a radiolabel or a fluorescent tag, or can be mass differentiated for detection by mass spectrometry or other means.

Also provided is an isolated nucleic acid molecule that includes the sequence of molecules that is complementary to the nucleotide sequence encoding MTSP20 or the portion thereof. Double-stranded RNA (dsRNA), such as RNAi is also provided.

3. Plasmids, Vectors and Cells

Plasmids and vectors containing the nucleic acid molecules are also provided. Cells containing the vectors, including cells that express the encoded proteins are provided. The cell can be a bacterial cell, a yeast cell, a fungal cell, a plant cell, an insect cell or an animal cell. Methods for producing an MTSP or single chain form (or multi-chain form) of a protease domain thereof by, for example, growing the cell under conditions whereby the encoded MTSP is expressed by the cell, and recovering the expressed protein, are provided herein. As noted, for MTSP20, the full-length zymogens and activated proteins and activated (two, three or multi-chain) protease and single chain protease domains are provided. As described herein, the cells are used for expression of the protein, which can be secreted or expressed in the cytoplasm.

As discussed below, the MTSP20 polypeptide, and catalytically active portions thereof, can be expressed on the surface of a cell. In addition, all or portions thereof can be expressed as a secreted protein using the native signal sequence or a heterologous signal. Alternatively, all or portions of the polypeptide can be expressed as inclusion bodies in the cytoplasm and isolated therefrom. The resulting protein can be treated to refold if necessary.

The above discussion provides an overview and some details of the exemplified MTSP20s.

C. Tumor Specificity and Tissue Expression Profiles

MTSPs are of interest because they appear to be expressed and/or activated at different levels in tumor cells from normal cells, or have functional activity that is different in tumor cells from normal cells, such as by an alteration in a substrate for the MTSP, or a cofactor or receptor of the MTSP. MTSP20 is of interest because it is expressed or is active in tumor cells. Hence the MTSPs provided herein can serve as diagnostic markers for certain tumors.

Each MTSP has a characteristic tissue expression profile; the MTSPs in particular, although not exclusively expressed or activated in tumors, exhibit characteristic tumor tissue expression or activation profiles. In some instances, MTSPs can have different activity in a tumor cell from a non-tumor cell by virtue of a change in a substrate or cofactor or receptor therefor or other factor that would alter the functional activity of the MTSP. Hence each can serve as a diagnostic marker for particular tumors, by virtue of a level of activity and/or expression or function in a subject (i.e. a mammal, particularly a human) with neoplastic disease, compared to a subject or subjects that do not have the neoplastic disease. In addition, detection of activity (and/or expression) in a particular tissue can be indicative of neoplastic disease. Shed MTSPs in body fluids can be indicative of neoplastic disease. Also, by virtue of an activity and/or expression profiles of each, they can serve as therapeutic targets, such as by administration of modulators of an activity thereof, or, as by administration of a prodrug specifically activated by one of the MTSPs.

Tissue Expression Profiles

MTSP20

MTSP20 is expressed in many tissues. The MTSP20 transcript is found in liver, lymph node, cerebellum, pancreas, prostate, uterus, testis, glands (adrenal, thyroid and salivary), thymus, kidney and spleen. Lower transcript level is found in lung, placenta, bladder, ovary, digestive system, circulatory system and other parts of the brain. The MTSP20 transcript is found at a low level in a number of normal tissues including breast, prostate, cervix, uterus, ovary, colon, lung, small intestine, stomach, kidney, pancreas, thyroid and rectum. MTSP20 is also expressed in certain tumor cell lines including lung carcinoma (A519), colorectal carcinoma (SW480), lymphoma (Raji and Daudi), cervical carcinoma (HeLaS3) and leukemia (HL-60, K-562 and MOLT-4) cell lines. MTSP20 is expressed in endothelial cells; hence it is an endotheliase.

D. Identification and Isolation of MTSP20 Polypeptide Genes

The MTSP polypeptides and/or domains thereof, can be obtained by methods well known in the art for protein purification and recombinant protein expression. Any method known to those of skill in the art for identification of nucleic acids that encode desired genes can be used. Any method available in the art can be used to obtain a full-length (i.e., encompassing the entire coding region) cDNA or genomic DNA clone encoding an MTSP polypeptide. For example, the polymerase chain reaction (PCR) can be used to amplify a sequence that is expressed in normal and tumor cells or tissues, e.g., nucleic acids encoding an MTSP20 polypeptide (SEQ. Nos: 5 and 16), in a genomic or cDNA library. Oligonucleotide primers that hybridize to sequences at the 3′ and 5′ termini of the identified sequences can be used as primers to amplify by PCR sequences from a nucleic acid sample (RNA or DNA), generally a cDNA library, from an appropriate source (e.g., tumor or cancer tissue).

PCR can be carried out, e.g., by use of a Perkin-Elmer Cetus thermal cycler and Taq polymerase (Gene Amp™). The DNA being amplified can include mRNA or cDNA or genomic DNA from any eukaryotic species. One can choose to synthesize several different degenerate primers, for use in the PCR reactions. It is also possible to vary the stringency of hybridization conditions used in priming the PCR reactions, to amplify nucleic acid homologs (e.g., to obtain MTSP polypeptide sequences from species other than humans or to obtain human sequences with homology to MTSP20 polypeptide) by allowing for greater or lesser degrees of nucleotide sequence similarity between the known nucleotide sequence and the nucleic acid homolog being isolated. For cross-species hybridization, low stringency to moderate stringency conditions are used. For same species hybridization, moderately stringent to highly stringent conditions are used. The conditions can be empirically determined.

After successful amplification of the nucleic acid containing all or a portion of the identified MTSP polypeptide sequence or of a nucleic acid encoding all or a portion of an MTSP polypeptide homolog, that segment can be molecularly cloned and sequenced, and used as a probe to isolate a complete cDNA or genomic clone. This, in turn, permits the determination of the gene's complete nucleotide sequence, the analysis of its expression, and the production of its protein product for functional analysis. Once the nucleotide sequence is determined, an open reading frame encoding the MTSP polypeptide gene protein product can be determined by any method well known in the art for determining open reading frames, for example, using publicly available computer programs for nucleotide sequence analysis. Once an open reading frame is defined, it is routine to determine the amino acid sequence of the protein encoded by the open reading frame. In this way, the nucleotide sequences of the entire MTSP polypeptide genes as well as the amino acid sequences of MTSP polypeptide proteins and analogs can be identified.

Any eukaryotic cell potentially can serve as the nucleic acid source for the molecular cloning of the MTSP polypeptide gene. The nucleic acids can be isolated from vertebrate, mammalian, human, porcine, bovine, feline, avian, equine, canine, as well as additional primate sources, insects, plants and other organisms. The DNA can be obtained by standard procedures known in the art from cloned DNA (e.g., a DNA “library”), by chemical synthesis, by cDNA cloning, or by the cloning of genomic DNA, or fragments thereof, purified from the desired cell (see, e.g., Sambrook et al. (2001) Molecular Cloning, A Laboratory Manual, 3d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II). Clones derived from genomic DNA can contain regulatory and intron DNA regions in addition to coding regions; clones derived from cDNA will contain only exon sequences. For any source, the gene is cloned into a suitable vector for propagation thereof.

In the molecular cloning of the gene from genomic DNA, DNA fragments are generated, some of which will encode the desired gene. The DNA can be cleaved at specific sites using various restriction enzymes. Alternatively, one can use DNAse in the presence of manganese to fragment the DNA, or the DNA can be physically sheared, for example, by sonication. The linear DNA fragments then can be separated according to size by standard techniques, including but not limited to, agarose and polyacrylamide gel electrophoresis and column chromatography.

Once the DNA fragments are generated, identification of the specific DNA fragment containing the desired gene can be accomplished in a number of ways. For example, a portion of the MTSP polypeptide (of any species) gene (e.g., a PCR amplification product obtained as described above or an oligonucleotide having a sequence of a portion of the known nucleotide sequence) or its specific RNA, or a fragment thereof can be purified and labeled, and the generated DNA fragments can be screened by nucleic acid hybridization to the labeled probe (Benton and Davis, Science 196:180 (1977); Grunstein and Hogness, Proc. Natl. Acad. Sci. U.S.A. 72:3961 (1975)). Those DNA fragments with substantial homology to the probe will hybridize. It is also possible to identify the appropriate fragment by restriction enzyme digestion(s) and comparison of fragment sizes with those expected according to a known restriction map if such is available or by DNA sequence analysis and comparison to the known nucleotide sequence of MTSP polypeptide. Further selection can be carried out on the basis of the properties of the gene. Alternatively, the presence of the gene can be detected by assays based on the physical, chemical, or immunological properties of its expressed product. For example, cDNA clones, or DNA clones which hybrid-select the proper mRNA, can be selected which produce a protein that, e.g., has similar or identical electrophoretic migration, isolectric focusing behavior, proteolytic digestion maps, antigenic properties, serine protease activity. If an anti-MTSP polypeptide antibody is available, the protein can be identified by binding of labeled antibody to the putatively MTSP polypeptide synthesizing clones, in an ELISA (enzyme-linked immunosorbent assay)-type procedure.

Alternatives to isolating the MTSP20 polypeptide genomic DNA include, but are not limited to, chemically synthesizing the gene sequence from a known sequence or making cDNA to the mRNA that encodes the MTSP polypeptide. For example, RNA for cDNA cloning of the MTSP polypeptide gene can be isolated from cells expressing the protein. The identified and isolated nucleic acids then can be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the Bluescript vector (Stratagene, La Jolla, Calif.). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can include specific chemically synthesized oligonucleotides encoding restriction endonuclease recognition sequences. In an alternative method, the cleaved vector and MTSP polypeptide gene can be modified by homopolymeric tailing. Recombinant molecules can be introduced into host cells via transformation, transfection, infection, electroporation, calcium precipitation and other methods, so that many copies of the gene sequence are generated.

In specific embodiments, transformation of host cells with recombinant DNA molecules that incorporate the isolated MTSP polypeptide gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA. Identification and isolation of nucleic acid encoding an exemplary MTSP20 is described in the EXAMPLES.

E. Vectors, Plasmids and Cells that Contain Nucleic Acids Encoding an MTSP Polypeptide or Protease Domain Thereof and Expression of MTSP Polypeptides

Vectors and Cells

For recombinant expression of one or more of the MTSP polypeptides, the nucleic acid containing all or a portion of the nucleotide sequence encoding the MTSP polypeptide can be inserted into an appropriate expression vector, i.e., a vector that contains the necessary elements for the transcription and translation of the inserted protein coding sequence. The necessary transcriptional and translational signals can also be supplied by the native promoter for MTSP genes, and/or their flanking regions.

Also provided are vectors that contain nucleic acid encoding the MTSPs. Cells containing the vectors are also provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, plant cells, insect cells and animal cells. The cells are used to produce an MTSP polypeptide or protease domain thereof by (a) growing the above-described cells under conditions whereby the encoded MTSP polypeptide or a protease domain of the MTSP polypeptide is expressed by the cell, and then (b) recovering the expressed polypeptide.

In one embodiment, vectors that include a sequence of nucleotides that encode a polypeptide that has protease activity and contains all or a portion of only a protease domain, or multiple copies thereof or a fragment with two protease domains, of an MTSP20 polypeptide are provided. Also provided are vectors that include a sequence of nucleotides that encodes a protease domain and additional portions of an MTSP protein up to and including a full length SP protein, as well as multiple copies thereof. The vectors can be selected for expression of the SP protein or protease domain thereof in the cell or such that the SP protein is expressed as a secreted protein. Alternatively, the vectors can include signals necessary for secretion of encoded proteins. When a protease domain is expressed the nucleic acid is linked to nucleic acid encoding a secretion signal, such as the Saccharomyces cerevisiae a mating factor signal sequence or a portion thereof, or the native signal sequence.

A variety of host-vector systems can be used to express the protein coding sequence. These include but are not limited to mammalian cell systems infected with virus (e.g. vaccinia virus, adenovirus, etc.); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of nucleic acid fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding MTSP polypeptide, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for MTSP polypeptide. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Villa-Kamaroff et al., Proc. Natl. Acad. Sci. USA 75:3727-3731 1978)) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthetase promoter (Herrar-Estrella et al., Nature 303:209-213 (1984)) or the cauliflower mosaic virus 35S RNA promoter (Garder et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Mogram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Sani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding an MTSP polypeptide, or a domain, fragment, derivative or homolog, thereof, one or more origins of replication, and optionally, one or more selectable markers (e.g., an antibiotic resistance gene). Expression vectors containing the coding sequences, or portions thereof, of an MTSP polypeptide, is made, for example, by subcloning the coding portions into the EcoRI restriction site of each of the three pGEX vectors (glutathione S-transferase expression vectors (Smith and Johnson, Gene 7:31-40 (1988)). This allows for the expression of products in the correct reading frame. Exemplary vectors and systems for expression of the protease domains of the MTSP20 polypeptides include the well-known Pichia vectors (available, for example, from Invitrogen, San Diego, Calif.), particularly those designed for secretion of the encoded proteins. The protein can also be expressed cytoplasmically, such as in the inclusion bodies. One exemplary vector is described in the EXAMPLES.

Plasmids for transformation of E. coli cells, include, for example, the pET expression vectors (see, U.S. Pat. No. 4,952,496; available from NOVAGEN, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (NOVAGEN, Madison, Wis.), which contain a His-Tag™ leader sequence for use in purification with a His column and a thrombin cleavage site that permits cleavage following purification over the column; the T7-lac promoter region and the T7 terminator.

The vectors are introduced into host cells, such as Pichia cells and bacterial cells, such as E. coli, and the proteins expressed therein. Exemplary Pichia strains, include, for example, GS115. Exemplary bacterial hosts contain chromosomal copies of DNA encoding T7 RNA polymerase operably linked to an inducible promoter, such as the lacUV promoter (see, U.S. Pat. No. 4,952,496). Such hosts include, but are not limited to, the lysogenic E. coli strain BL21 (DE3).

Expression and Production of Proteins

The MTSP domains, derivatives and analogs can be produced by various methods known in the art. For example, once a recombinant cell expressing an MTSP polypeptide, or a domain, fragment or derivative thereof, is identified, the individual gene product can be isolated and analyzed. This is achieved by assays based on the physical and/or functional properties of the protein, including, but not limited to, radioactive labeling of the product followed by analysis by gel electrophoresis, immunoassay, cross-linking to marker-labeled product, and assays of proteolytic activity.

The MTSP polypeptides can be isolated and purified by standard methods known in the art (either from natural sources or recombinant host cells expressing the complexes or proteins), including but not restricted to column chromatography (e.g., ion exchange, affinity, gel exclusion, reversed-phase high pressure and fast protein liquid), differential centrifugation, differential solubility, or by any other standard technique used for the purification of proteins. Functional properties can be evaluated using any suitable assay known in the art.

Alternatively, once an MTSP polypeptide or its domain or derivative is identified, the amino acid sequence of the protein can be deduced from the nucleotide sequence of the gene which encodes it. As a result, the protein or its domain or derivative can be synthesized by standard chemical methods known in the art (e.g. see Hunkapiller et al, Nature 310:105-111 (1984)).

Manipulations of MTSP polypeptide sequences can be made at the protein level. Also contemplated herein are MTSP polypeptide proteins, domains thereof, derivatives or analogs or fragments thereof, which are differentially modified during or after translation, e.g., by glycosylation, acetylation, phosphorylation, amidation, derivatization by known protecting/blocking groups, proteolytic cleavage, linkage to an antibody molecule or other cellular ligand. Any of numerous chemical modifications can be carried out by known techniques, including but not limited to specific chemical cleavage by cyanogen bromide, trypsin, chymotrypsin, papain, V8 protease, NaBH₄, acetylation, formylation, oxidation, reduction, metabolic synthesis in the presence of tunicamycin and other such agents.

In addition, domains, analogs and derivatives of an MTSP polypeptide can be chemically synthesized. For example, a peptide corresponding to a portion of an MTSP polypeptide, which includes the desired domain or which mediates the desired activity in vitro can be synthesized by use of a peptide synthesizer. Furthermore, if desired, nonclassical amino acids or chemical amino acid analogs can be introduced as a substitution or addition into the MTSP polypeptide sequence. Non-classical amino acids include but are not limited to the D-isomers of the common amino acids, a-amino isobutyric acid, 4-aminobutyric acid, Abu, 2-aminobutyric acid, ε-Abu, e-Ahx, 6-amino hexanoic acid, Aib, 2-amino isobutyric acid, 3-amino propionoic acid, ornithine, norleucine, norvaline, hydroxyproline, sarcosine, citrulline, cysteic acid, t-butylglycine, t-butylaianine, phenylglycine, cyclohexylalanine, β-alanine, fluoro-amino acids, designer amino acids such as R-methyl amino acids, Ca-methyl amino acids, Na-methyl amino acids, and amino acid analogs in general. Furthermore, the amino acid can be D (dextrorotary) or L (levorotary).

In cases where natural products are suspected of being mutant or are isolated from new species, the amino acid sequence of the MTSP polypeptide isolated from the natural source, as well as those expressed in vitro, or from synthesized expression vectors in vivo or in vitro, can be determined from analysis of the DNA sequence, or alternatively, by direct sequencing of the isolated protein. Such analysis can be performed by manual sequencing or through use of an automated amino acid sequenator.

Modifications

A variety of modifications of the MTSP polypeptides and domains are contemplated herein. An MTSP-encoding nucleic acid molecule can be modified by any of numerous strategies known in the art (Sambrook et al. (1990), Molecular Cloning, A Laboratory Manual, 2d ed., Cold Spring Harbor Laboratory, Cold Spring Harbor, N.Y.). The sequences can be cleaved at appropriate sites with restriction endonuclease(s), followed by further enzymatic modification if desired, isolated, and ligated in vitro. In the production of the gene encoding a domain, derivative or analog of MTSP, care should be taken to ensure that the modified gene retains the original translational reading frame, uninterrupted by translational stop signals, in the gene region where the desired activity is encoded.

Additionally, the MTSP-encoding nucleic acid molecules can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Also, as described herein muteins with primary sequence alterations, such as replacements of Cys residues and elimination or addition of glycosylation sites are contemplated; the MTSP20 that includes the sequence of amino acids set forth in SEQ ID No. 6 has potential glycosylation sites at . . . N₇₇QS . . . and . . . N₆₂₁IS (SEQ ID No. 16). Mutations can be effected by any technique for mutagenesis known in the art, including, but not limited to, chemical mutagenesis and in vitro site-directed mutagenesis (Hutchinson et al., J. Biol. Chem. 253:6551-6558 (1978)), use of TAB® linkers (Pharmacia). In one embodiment, for example, an MTSP polypeptide or domain thereof is modified to include a fluorescent label. In other specific embodiments, the MTSP polypeptide is modified such that heterobifunctional reagents can be used to crosslink the members of a complex.

In addition, domains, analogs and derivatives of an MTSP can be chemically synthesized. For example, a peptide corresponding to a portion of an MTSP, which includes the desired domain or which mediates the desired activity in vitro can be synthesized by use of a peptide synthesizer. Furthermore, if desired, nonclassical amino acids or chemical amino acid analogs can be introduced as a substitution or addition into the MTSP sequence. Non-classical amino acids include but are not limited to the D-isomers of the common amino acids, a-amino isobutyric acid, 4-aminobutyric acid, Abu, 2-aminobutyric acid, ε-Abu, e-Ahx, 6-amino hexanoic acid, Aib, 2-amino isobutyric acid, 3-amino propionoic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general. Furthermore, the amino acid can be D (dextrorotary) or L (levorotary).

F. Screening Methods

The single chain protease domains, as shown herein, can be used in a variety of methods to identify compounds that modulate an activity thereof. For SPs that exhibit higher activity or expression in tumor cells or angiogenic endothelial cells, compounds that inhibit the proteolytic activity are of particular interest. For any SPs that are active at lower levels in tumor cells or quiescent endothelial cells, compounds or agents that enhance an activity are potentially of interest. In all instances the identified compounds include agents that are candidate cancer treatments.

Several types of assays are exemplified and described herein. It is understood that a protease domain or domains can be used in other assays. The single chain protease domains exhibit catalytic activity. As such they are suitable for in vitro screening assays. They can also be used in binding assays.

The MTSP20 full length zymogens, activated enzymes, single and multi-chain protease domains are contemplated for use in any screening assay known to those of skill in the art, including those provided herein. Hence the following description, if directed to proteolytic assays is intended to apply to use of a single chain protease domain or a catalytically active portion thereof of any SP, including an MTSP20. Other assays, such as binding assays are provided herein, particularly for use with an MTSP20, including any variants, such as splice variants thereof.

1. Catalytic Assays for Identification of Agents that Modulate the Protease Activity of an MTSP Protein

Methods for identifying a modulator of the catalytic activity of an SP, particularly a single chain protease domain or catalytically active portion thereof, are provided herein. The methods can be practiced by: contacting the MTSP20, a full-length zymogen or activated form, and particularly a single-chain domain thereof, with a substrate of the MTSP20 in the presence of a test substance, and detecting the proteolysis of the substrate, whereby an activity of the MTSP20 is assessed, and comparing the activity to a control. For example, a control can be the activity of the MTSP20 assessed by contacting an MTSP20, including a full-length zymogen or activated form, and particularly a single-chain domain thereof, with a substrate of the MTSP20, and detecting the proteolysis of the substrate, whereby an activity of the MTSP20 is assessed. The results in the presence and absence of the test compounds are compared. A difference in an activity indicates that the test substance modulates an activity of the MTSP20. Modulators, including activators and inhibitors, of MTSP20 activation cleavage are also contemplated; such assays are discussed below.

In one embodiment a plurality of the test substances are screened simultaneously in the above screening method. In another embodiment, the MTSP20 is isolated from a target cell as a means for then identifying agents that are potentially specific for the target cell.

In another embodiment, a test substance is a therapeutic compound, whereby a difference of the MTSP20 activity measured in the presence and in the absence of the test substance indicates that the target cell responds to the therapeutic compound.

One method includes the steps of (a) contacting the MTSP20 polypeptide or protease domain thereof with one or a plurality of test compounds under conditions conducive to interaction between the ligand and the compounds; and (b) identifying one or more compounds in the plurality that specifically binds to the ligand.

Another method provided herein includes the steps of a) contacting an MTSP20 polypeptide or protease domain thereof with a substrate of the MTSP20 polypeptide, and detecting the proteolysis of the substrate, whereby the activity of an MTSP20 polypeptide is assessed; b) contacting the MTSP20 polypeptide with a substrate of the MTSP20 polypeptide in the presence of a test substance, and detecting the proteolysis of the substrate, whereby an activity of the MTSP20 polypeptide is assessed; and c) comparing the activity of the MTSP20 polypeptide assessed in steps a) and b), whereby the activity measured in step a) differs from the activity measured in step b) indicates that the test substance modulates an activity of the MTSP20 polypeptide.

In another embodiment, a plurality of the test substances are screened simultaneously. In comparing an activity of an MTSP20 polypeptide in the presence and absence of a test substance to assess whether the test substance is a modulator of the MTSP20 polypeptide, it is unnecessary to assay the activity in parallel, although such parallel measurement is typical. It is possible to measure an activity of the MTSP20 polypeptide at one time point and compare the measured activity to a historical value of an activity of the MTSP20 polypeptide.

For instance, one can measure an activity of the MTSP20 polypeptide in the presence of a test substance and compare with historical value of an activity of the MTSP20 polypeptide measured previously in the absence of the test substance, and vice versa. This can be accomplished, for example, by providing the activity of the MTSP20 polypeptide on an insert or pamphlet provided with a kit for conducting the assay.

Methods for selecting substrates for a particular SP are described in the EXAMPLES, and particular proteolytic assays are exemplified.

Combinations and kits containing the combinations optionally including instructions for performing the assays are provided. The combinations include an MTSP20 polypeptide and a substrate of the MTSP20 polypeptide to be assayed; and, optionally reagents for detecting proteolysis of the substrate. The substrates, which can be chromogenic or fluorogenic molecules, including proteins, subject to proteolysis by a particular MTSP20 polypeptide, can be identified empirically by testing the ability of the MTSP20 polypeptide to cleave the test substrate. Substrates that are cleaved most effectively (i.e., at the lowest concentrations and/or fastest rate or under desirable conditions), are identified.

Additionally provided herein is a kit containing the above-described combination. The kit optionally includes instructions for identifying a modulator of an activity of an MTSP20 polypeptide. Any MTSP20 polypeptide is contemplated as target for identifying modulators of an activity thereof.

2. Binding Assays

Also provided herein are methods for identification and isolation of agents, particularly compounds that bind to MTSP20s. The assays are designed to identify agents that bind to the zymogen form, a single chain isolated protease domain (or a protein, other than an MTSP20 polypeptide, that contains a protease domain of an MTSP20 polypeptide), and to the activated form, including the activated form derived from the full length zymogen or from an extended protease domain. The identified compounds are candidates or leads for identification of compounds for treatments of tumors and other disorders and diseases involving aberrant angiogenesis. The MTSP20 polypeptides used in the methods include any MTSP20 polypeptide as defined herein, including the MTSP20 single chain protease domain or proteolytically active portion thereof.

A variety of methods are provided herein. These methods can be performed in solution or in solid phase reactions in which the MTSP20 polypeptide(s) or protease domain(s) thereof are linked, either directly or indirectly via a linker, to a solid support. Screening assays are described in the Examples, and these assays have been used to identify candidate compounds. For purposes herein, all binding assays described above are provided for MTSP20.

Methods for identifying an agent, such as a compound, that specifically binds to an MTSP20 single and/or two chain protease domain, a zymogen and/or full-length activated multi-chain (two, three or more chains) MTSP20 or two chain protease domain thereof are provided herein. The method can be practiced by (a) contacting the MTSP20 with one or a plurality of test agents under conditions conducive to binding between the MTSP20 and an agent; and (b) identifying one or more agents within the plurality that specifically binds to the MTSP20.

For example, in practicing such methods the MTSP20 polypeptide is mixed with a potential binding partner or an extract or fraction of a cell or tissue under conditions that allow the association of potential binding partners with the polypeptide. After mixing, peptides, polypeptides, proteins or other molecules that have become associated with an MTSP20 are separated from the mixture. The binding partner that bound to the MTSP20 can then be removed and further analyzed. To identify and isolate a binding partner, the entire protein, for instance the entire polypeptide of SEQ ID No. 6 can be used. Alternatively, a fragment of the protein can be used.

A variety of methods can be used to obtain cell or tissue extracts or body fluids, such as blood, serum, urine, sweat, synovial fluid, CSF and other such fluids. For example, cells and tissues can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can adapt methods for preparing cellular or tissue extracts in order to obtain extracts for use in the present methods.

Once an extract of a cell or tissue is prepared, the extract is mixed with the MTSP20 under conditions in which association of the protein with the binding partner can occur. A variety of conditions can be used, including conditions that resemble conditions found in the cytoplasm of a human cell, in a tissue, such as a tumor, or in a body fluid, such as blood. Features, such as osmolarity, pH, temperature, and the concentration of extract used, can be varied to optimize the association of the protein with the binding partner. Similarly, methods for isolation of molecules of interest from body fluids are known.

After mixing under appropriate conditions, the bound complex is separated from the mixture. A variety of techniques can be used to separate the mixture. For example, antibodies specific to an MTSP20 can be used to immunoprecipitate the binding partner complex. Alternatively, standard chemical separation techniques such as chromatography and density/sediment centrifugation can be used. After removing the non-associated cellular constituents in the extract, the binding partner can be dissociated from the complex using conventional methods. For example, dissociation can be accomplished by altering the salt concentration or pH of the mixture.

To aid in separating associated binding partner pairs from the mixed extract, the MTSP20 can be immobilized on a solid support. For example, the protein can be attached to a nitrocellulose matrix or acrylic beads. Attachment of the protein or a fragment thereof to a solid support aids in separating peptide/binding partner pairs from other constituents found in the extract. Alternatively MTSP20 conjugates, such as those provided herein, can be used to separte binding partners from an extract. The identified binding partners can be either a single protein or a complex made up of two or more proteins.

Alternatively, the nucleic acid molecules encoding the single chain proteases can be used in a yeast two-hybrid system. The yeast two-hybrid system has been used to identify other protein partner pairs and can readily be adapted to employ the nucleic acid molecules herein described.

Another in vitro binding assay, particularly for an MTSP20, uses a mixture of a polypeptide that contains at least the catalytic domain of one of these proteins and one or more candidate binding targets or substrates. After incubating the mixture under appropriate conditions, the ability of the MTSP20 or a polypeptide fragment thereof containing the catalytic domain to bind to or interact with the candidate substrate is assessed. For cell-free binding assays, one of the components includes or is coupled to a detectable label. The label can provide for direct detection, such as radioactivity, luminescence, optical or electron density, etc., or indirect detection such as an epitope tag, an enzyme, etc. A variety of methods can be employed to detect the label depending on the nature of the label and other assay components. For example, the label can be detected bound to the solid substrate or a portion of the bound complex containing the label can be separated from the solid substrate, and the label thereafter detected.

3. Detection of Signal Transduction

MTSP20, which is a transmembrane protein, can be involved directly or indirectly in signal transduction directly as a cell surface receptor or indirectly by activating proteins, such as pro-growth factors that can initiate signal transduction.

In addition, secretion of MTSP20, such as an extracellular domain or region of MTSP20, can be involved in signal transduction either directly by binding to or interacting with a cell surface receptor or indirectly by activating proteins, such as pro-growth factors that can initiate signal transduction. Assays for assessing signal transduction are well known to those of skill in the art, and can be adapted for use with the MTSP20 polypeptide.

Assays for identifying agents that affect or alter signal transduction mediated directly or indirectly, such as via activation of a pro-growth factor, by an MTSP20, particularly the full length or a sufficient portion to anchor the extracellular domain or a functional portion thereof of an MTSP20 on the surface of a cell are provided. Such assays, include, for example, transcription based assays in which modulation of a transduced signal is assessed by detecting an effect on an expression from a reporter gene (see, e.g., U.S. Pat. No. 5,436,128).

4. Methods for Identifying Agents that Modulate the Expression a Nucleic Acid Encoding an MTSP20

Another embodiment provides methods for identifying agents that modulate the expression of a nucleic acid encoding an MTSP20. Such assays use any available means of monitoring for changes in the expression level of the nucleic acids encoding an MTSP20.

In one assay format, cell lines that contain reporter gene fusions between the open reading frame of MTSP20 or a domain thereof, particularly a protease domain and any assayable fusion partner can be prepared. Numerous assayable fusion partners are known and readily available including the firefly luciferase gene and the gene encoding chloramphenicol acetyltransferase (Alam et al., Anal. Biochem. 188: 245-54 (1990)). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding an MTSP20.

Additional assay formats can be used to monitor the ability of the agent to modulate the expression of a nucleic acid encoding an MTSP20. For instance, mRNA expression can be monitored directly by hybridization to the nucleic acids. Cell lines are exposed to the agent to be tested under appropriate conditions and time and total RNA or mRNA is isolated by standard procedures (see, e.g., Sambrook et al. (1989) MOLECULAR CLONING: A LABORATORY MANUAL, 2nd Ed. Cold Spring Harbor Laboratory Press). Probes to detect differences in RNA expression levels between cells exposed to the agent and control cells can be prepared from the nucleic acids. It is typical, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes can be designed from the nucleic acids through methods known in the art. For instance, the G+C content of the probe and the probe length can affect probe binding to its target sequence. Methods to optimize probe specificity are commonly available (see, e.g., Sambrook et al. (1989) MOLECULAR CLONING: A LABORATORY MANUAL, 2nd Ed. Cold Spring Harbor Laboratory Press); and Ausubel et al. (1995) CURRENT PROTOCOLS IN MOLECULAR BIOLOGY, Greene Publishing Co., NY).

Hybridization conditions are modified using known methods (see, e.g., Sambrook et al. (1989) MOLECULAR CLONING: A LABORATORY MANUAL, 2nd Ed. Cold Spring Harbor Laboratory Press); and Ausubel et al. (1995) CURRENT PROTOCOLS IN MOLECULAR BIOLOGY, Greene Publishing Co., NY), as required for each probe. Hybridization of total cellular RNA or RNA enriched for polyA RNA can be accomplished in any available format. For instance, total cellular RNA or RNA enriched for polyA RNA can be affixed to a solid support, and the solid support exposed to at least one probe comprising at least one, or part of one of the nucleic acid molecules under conditions in which the probe specifically hybridizes. Alternatively, nucleic acid fragments comprising at least one, or part of one of the sequences can be affixed to a solid support, such as a porous glass wafer. The glass wafer can then be exposed to total cellular RNA or polyA RNA from a sample under conditions in which the affixed sequences specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding the MTSP20 polypeptide, are identified.

In one format, the relative amounts of a protein between a cell population that has been exposed to the agent to be tested compared to an un-exposed control cell population can be assayed (e.g., a prostate cancer cell line, a lung cancer cell line, a colon cancer cell line or a breast cancer cell line). In this format, probes, such as specific antibodies, are used to monitor the differential expression or level of activity of the protein in the different cell populations or body fluids. Cell lines or populations, tissues or body fluids are exposed to the agent to be tested under appropriate conditions and time. Cellular or tissue lysates or body fluids can be prepared from the exposed cell line or tissue or population and a control, unexposed cell line or population, or tissue or unexposed body fluid. The lysates or body fluids are then analyzed with the probe.

For example, N- and C-terminal fragments of the MTSP20 can be expressed in bacteria and used to search for proteins which bind to these fragments. Fusion proteins, such as His-tag or GST fusion to the N- or C-terminal regions of the MTSP20 can be prepared for use as a substrate. These fusion proteins can be coupled to, for example, Glutathione-Sepharose beads and then probed with tissue or cell lysates or body fluids. Prior to lysis, the tissues, cells or body fluids can be treated with a candidate agent which can modulate an MTSP20 or proteins that interact with domains thereon. Lysate proteins binding to the fusion proteins can be resolved by SDS-PAGE, isolated and identified by protein sequencing or mass spectroscopy, as is known in the art.

Antibody probes are prepared by immunizing suitable mammalian hosts in appropriate immunization protocols using the peptides, polypeptides or proteins if they are of sufficient length (e.g., 4, 5, 6, 7, 8, 9, 10, 11, 12, 13, 14, 15, 20, 25, 30, 35, 40 or more consecutive amino acids the MTSP20 polypeptide) or if required to enhance immunogenicity, conjugated to suitable carriers. Methods for preparing immunogenic conjugates with carriers, such as bovine serum albumin (BSA), keyhole limpet hemocyanin (KLH), or other carrier proteins are well known in the art. In some circumstances, direct conjugation using, for example, carbodiimide reagents can be effective; in other instances linking reagents such as those supplied by Pierce Chemical Co., Rockford, Ill., can be desirable to provide accessibility to the hapten. Hapten peptides can be extended at either the amino or carboxy terminus with a Cys residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides corresponding to, for example, the carboxy terminal amino acids of the MTSP20. Synthetic peptides can be as small as 1-3 amino acids in length, generally at least 4 or more amino acid residues long. The peptides can be coupled to KLH using standard methods and can be immunized into animals, such as rabbits or ungulates. Polyclonal antibodies can then be purified, for example using Actigel beads containing the covalently bound peptide.

While the polyclonal antisera produced in this way can be satisfactory for some applications, for pharmaceutical compositions, use of monoclonal preparations are generally used. Immortalized cell lines which secrete the desired monoclonal antibodies can be prepared using the standard method of Kohler et al., (Nature 256: 495-7 (1975)) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in vivo via ascites fluid. Of particular interest, are monoclonal antibodies that recognize a catalytic domain or an activation cleavage site region(s) of an MTSP20.

Additionally, the zymogen or a multi-chain form of the MTSP20 can be used to make monoclonal antibodies that recognize conformation epitopes. The desired monoclonal antibodies are then recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as the Fab, Fab′, of F(ab′)₂ fragments are often used, especially in a therapeutic context, as these fragments are generally less immunogenic than the whole immunoglobulin. The antibodies or fragments can also be produced. Regions of antibodies or fragments that bind specifically to the desired epitopes of MTSP20 polypeptides also can be produced in the context of chimeras with multiple species origin.

Agents that are assayed in the above method can be randomly selected or rationally selected or designed. The agents can be, as examples, peptides, small molecules, and carbohydrates. A skilled artisan can recognize that there is no limit as to the structural nature of the agents.

The peptide agents can be prepared using standard solid phase (or solution phase) peptide synthesis methods, as is known in the art. In addition, the peptides can be produced recombinantly from nucleic acid. The nucleic acid encoding these peptides can be synthesized using commercially available oligonucleotide synthesis instrumentation and produced recombinantly using standard recombinant production systems. The production using solid phase peptide synthesis is necessitated if non-gene-encoded amino acids are to be included.

G. Assay Formats and Selection of Test Substances that Modulate at Least One Activity of an MTSP20 Polypeptide

Methods for identifying agents that modulate at least one activity of an MTSP20 are provided. The methods include phage display and other methods for assessing alterations in an activity of an MTSP20. Such methods or assays can use any means of monitoring or detecting the desired activity. A variety of formats and detection protocols are known for performing screening assays. Any such formats and protocols can be adapted for identifying modulators of MTSP20 polypeptide activities. The following includes a discussion of exemplary protocols.

1. High Throughput Screening Assays

Although the above-described assay can be conducted where a single MTSP20 polypeptide is screened, and/or a single test substance is screened in one assay, the assay typically is conducted in a high throughput screening mode, i.e., a plurality of the SP proteins are screened against and/or a plurality of the test substances are screened simultaneously (See generally, High Throughput Screening: The Discovery of Bioactive Substances (Devlin, Ed.) Marcel Dekker, 1997; Sittampalam et al., Curr. Opin. Chem. Biol., 1:384-91 (1997); and Silverman et al., Curr. Opin. Chem. Biol., 2:397-403 (1998)). For example, the assay can be conducted in a multi-well (e.g., 24-, 48-, 96-, 384-, 1536-well or higher density), chip or array format.

High-throughput screening (HTS) is the process of testing a large number of diverse chemical structures against disease targets to identify “hits” (Sittampalam et al., Curr. Opin. Chem. Biol., 1:384-91 (1997)). Current state-of-the-art HTS operations are highly automated and computerized to handle sample preparation, assay procedures and the subsequent processing of large volumes of data.

Detection technologies employed in high-throughput screens depend on the type of biochemical pathway being investigated (Sittampalam et al., Curr. Opin. Chem. Biol., 1:384-91 (1997)). These methods include, radiochemical methods, such as the scintillation proximity assays (SPA), which can be adapted to a variety of enzyme assays (Lerner et al., J. Biomol. Screening, 1:135-143 (1996); Baker et al., Anal. Biochem., 239:20-24 (1996); Baum et al., Anal. Biochem., 237:129-134 (1996); and Sullivan et al., J. Biomol. Screening 2:19-23 (1997)) and protein-protein interaction assays (Braunwalder et al., J. Biomol. Screening 1:23-26 (1996); Sonatore et al., Anal. Biochem. 240:289-297 (1996); and Chen et al., J. Biol. Chem. 271:25308-25315 (1996)), and non-isotopic detection methods, including but not limited to, colorimetric and luminescence detection methods, resonance energy transfer (RET) methods, time-resolved fluorescence (HTRF) methods, cell-based fluorescence assays, such as fluorescence resonance energy transfer (FRET) procedures (see, e.g., Gonzalez et al., Biophys. J., 69:1272-1280 (1995)), fluorescence polarization or anisotropy methods (see, e.g., Jameson et al., Methods Enzymol. 246:283-300 (1995); Jolley, J. Biomol. Screening 1:33-38 (1996); Lynch et al., Anal. Biochem. 247:77-82 (1997)), fluorescence correlation spectroscopy (FCS) and other such methods.

2. Test Substances

Test compounds, including small molecules, antibodies, proteins, nucleic acids, peptides, natural products, extracts containing natural products and libraries and collections thereof, can be screened in the above-described assays and assays described below to identify compounds that modulate an activity of an MTSP20 polypeptide. Rational drug design methodologies that rely on computational chemistry can be used to screen and identify candidate compounds.

The compounds identified by the screening methods include inhibitors, including antagonists, and can be agonists. Compounds for screening include any compounds and collections of compounds available, known or that can be prepared.

a. Selection of Compounds

Compounds can be selected for their potency and selectivity of inhibition of serine proteases, especially an MTSP20 polypeptide. As described herein, and as generally known, a target serine protease and its substrate are combined under assay conditions permitting reaction of the protease with its substrate. The assay is performed in the absence of test compound, and in the presence of increasing concentrations of the test compound. The concentration of test compound at which 50% of the serine protease activity is inhibited by the test compound is the IC₅₀ value (Inhibitory Concentration) or EC₅₀ (Effective Concentration) value for that compound. Within a series or group of test compounds, those having lower IC₅₀ or EC₅₀ values are considered more potent inhibitors of the serine protease than those compounds having higher IC₅₀ or EC₅₀ values. The IC₅₀ measurement is often used for more simplistic assays, whereas the EC₅₀ is often used for more complicated assays, such as those employing cells.

Typically candidate compounds have an IC₅₀ value of 100 nM or less as measured in an in vitro assay for inhibition of MTSP20 polypeptide activity. The test compounds also are evaluated for selectivity toward a serine protease. As described herein, and as generally known, a test compound is assayed for its potency toward a panel of serine proteases and other enzymes and an IC₅₀ value or EC₅₀ value is determined for each test compound in each assay system. A compound that demonstrates a low IC₅₀ value or EC₅₀ value for the target enzyme, e.g., MTSP20 polypeptide, and a higher IC₅₀ value or EC₅₀ value for other enzymes within the test panel (e.g., urokinase tissue plasminogen activator, thrombin, Factor Xa), is considered to be selective toward the target enzyme. Generally, a compound is deemed selective if its IC₅₀ value or EC₅₀ value in the target enzyme assay is at least one order of magnitude less than the next smallest IC₅₀ value or EC₅₀ value measured in the selectivity panel of enzymes.

Compounds are also evaluated for their activity in vivo. The type of assay chosen for evaluation of test compounds depends on the pathological condition to be treated or prevented by use of the compound, as well as the route of administration to be evaluated for the test compound.

For instance, to evaluate an activity of a compound to reduce tumor growth through inhibition of MTSP20 polypeptide, the procedures described by Jankun et al., Canc. Res. 57:559-563 (1997) to evaluate PAI-1 can be employed. Briefly, the ATCC cell lines DU145 and LnCaP are injected into SCID mice. After tumors are established, the mice are given test compound according to a dosing regime determined from the compound's in vitro characteristics. The Jankun et al. compound was administered in water. Tumor volume measurements are taken twice a week for about five weeks. A compound is deemed active if an animal to which the compound was administered exhibited decreased tumor volume, as compared to animals receiving appropriate control compounds.

Another in vivo experimental model designed to evaluate the effect of p-aminobenzamidine, a swine protease inhibitor, on reducing tumor volume is described by Billström et al., Int. J. Cancer 61:542-547 (1995).

To evaluate the ability of a compound to reduce the occurrence of, or inhibit, metastasis, the procedures described, for example, by Kobayashi et al. Int. J. Canc. 57:727-733d (1994) can be employed. Briefly, a murine xenograft selected for high lung colonization potential is injected into C57B1/6 mice i.v. (experimental metastasis) or s.c. into the abdominal wall (spontaneous metastasis). Various concentrations of the compound to be tested can be admixed with the tumor cells in Matrigel prior to injection. Daily i.p. injections of the test compound are made either on days 1-6 or days 7-13 after tumor inoculation. The animals are sacrificed about three or four weeks after tumor inoculation, and the lung tumor colonies are counted. Evaluation of the resulting data permits a determination as to efficacy of the test compound, optimal dosing and route of administration.

An activity of the tested compounds toward decreasing tumor volume and metastasis can be evaluated, for example, in a model described in Rabbani et al. (1995) Int. J. Cancer 63:840-845 in which Mat LyLu tumor cells were injected into the flank of Copenhagen rats. The animals were implanted with osmotic minipumps to continuously administer various doses of test compound for up to three weeks. The tumor mass and volume of experimental and control animals were evaluated during the experiment, as were metastatic growths. Evaluation of the resulting data permits a determination as to efficacy of the test compound, optimal dosing, and route of administration. Some of these authors described a related protocol in Xing et al., Canc. Res. 57:3585-3593 (1997).

To evaluate the anti-angiogenesis activity of a compound, a rabbit cornea neovascularization model can be employed (see, e.g., Avery et al. (1990) Arch. Ophthalmol., 108:1474-1476). Avery et al. describes anesthetizing New Zealand albino rabbits and then making a central corneal incision and forming a radial corneal pocket. A slow release prostaglandin pellet was placed in the pocket to induce neovascularization. Test compound was administered i.p. for five days, at which time the animals were sacrificed. The effect of the test compound is evaluated by review of periodic photographs taken of the limbus, which can be used to calculate the area of neovascular response and, therefore, limbal neovascularization. A decreased area of neovascularization as compared with appropriate controls indicates the test compound was effective at decreasing or inhibiting neovascularization.

An angiogenesis model used to evaluate the effect of a test compound in preventing angiogenesis is described by Min et al. Canc. Res. 56:2428-2433 (1996). C57BL6 mice receive subcutaneous injections of a Matrigel mixture containing bFGF, as the angiogenesis-inducing agent, with and without the test compound. After five days, the animals are sacrificed and the Matrigel plugs, in which neovascularization can be visualized, are photographed. An experimental animal receiving Matrigel and an effective dose of test compound exhibits less vascularization than a control animal or an experimental animal receiving a less- or non-effective does of compound.

An in vivo system designed to test compounds for their ability to limit the spread of primary tumors is described by Crowley et al., Proc. Natl. Acad. Sci. 90:5021-5025 (1993). Nude mice are injected with tumor cells (PC3) engineered to express CAT (chloramphenicol acetyltransferase). Compounds to be tested for their ability to decrease tumor size and/or metastases are administered to the animals, and subsequent measurements of tumor size and/or metastatic growths are made. In addition, the level of CAT detected in various organs provides an indication of the ability of the test compound to inhibit metastasis; detection of less CAT in tissues of a treated animal versus a control animal indicates less CAT-expressing cells migrated to that tissue.

In vivo experimental models designed to evaluate the inhibitory potential of test serine protease inhibitors, using a tumor cell line F311 known to be highly invasive (see, e.g., Alonso et al. (1996) Breast Canc. Res. Treat. 40:209-223) are provided. Alonso describes in vivo studies for toxicity determination, tumor growth, invasiveness, spontaneous metastasis, experimental lung metastasis, and an angiogenesis assay.

The CAM model (chick embryo chorioallantoic membrane model; Ossowski (1988) J. Cell Biol. 107:2437-2445), provides another method for evaluating the inhibitory activity of a test compound. In the CAM model, tumor cells invade through the chorioallantoic membrane containing CAM (with tumor cells in the presence of several serine protease inhibitors results in less or no invasion of the tumor cells through the membrane). Thus, the CAM assay is performed with CAM and tumor cells in the presence and absence of various concentrations of test compound. The invasiveness of tumor cells is measured under such conditions to provide an indication of the compound's inhibitory activity. A compound having inhibitory activity correlates with less tumor invasion.

The CAM model is also used in a standard assay of angiogenesis (i.e., effect on formation of new blood vessels (Brooks et al. (1999) Methods in Molecular Biology 129:257-269). According to this model, a filter disc containing an angiogenesis inducer, such as basic fibroblast growth factor (bFGF) is placed onto the CAM. Diffusion of the cytokine into the CAM induces local angiogenesis, which can be measured in several ways such as by counting the number of blood vessel branch points within the CAM directly below the filter disc. The ability of identified compounds to inhibit cytokine-induced angiogenesis can be tested using this model. A test compound can either be added to the filter disc that contains the angiogenesis inducer, be placed directly on the membrane or be administered systemically. The extent of new blood vessel formation in the presence and/or absence of test compound can be compared using this model. The formation of fewer new blood vessels in the presence of a test compound would be indicative of anti-angiogenesis activity. Demonstration of anti-angiogenesis activity for inhibitors of an MTSP20 polypeptide indicates a role in angiogenesis for that SP protein.

b. Known Serine Protease Inhibitors

Compounds for screening can be serine protease inhibitors, which can be tested for their ability to inhibit an activity of an MTSP20. Exemplary serine protease inhibitors for use in the screening assays include, but are not limited to: Serine Protease Inhibitor 3 (SPI-3) (Chen, et al. Citokine, 11:856-862 (1999)); Aprotinin (Iijima et al. J. Biochem. (Tokyo) 126:912-916 (1999)); Kazal-type serine protease inhibitor-like proteins (Niimi, et al. Eur. J. Biochem., 266:282-292 (1999)); Kunitz-type serine protease inhibitor (Ravichandran, S., et al., Acta Crystallogr. D. Biol. Crystallogr., 55:1814-1821 (1999)); Tissue factor pathway inhibitor-2/Matrix-associated serine protease inhibitor (TFPI-2/MSPI), (Liu, Y. et al. Arch. Biochem. Biophys. 370:112-8 (1999)); Bukunin (Cui, C. Y. et al. J. Invest. Dermatol. 113:182-8 (1999)); Nafmostat mesilate (Ryo, R. et al. (1999) Vox Sang. 76:241-6); TPCK (Huang et al. (1999) Oncogene 18:3431-3439); A synthetic cotton-bound serine protease inhibitor (Edwards (1999) et al. Wound Repair Regen. 7:106-18); FUT-175 (Sawada (1999) et al. Stroke 30:644-50); Combination of serine protease inhibitor FUT-0175 and thromboxane synthetase inhibitor OKY-046 (Kaminogo et al. (1998) Neurol. Med. Chir. (Tokyo) 38:704-8; discussion 708-9); the rat serine protease inhibitor 2.1 gene (LeCam, A., et al., Biochem. Biophys. Res. Commun., 253:311-4 (1998)); A new intracellular serine protease inhibitor expressed in the rat pituitary gland complexes with granzyme B (Hill et al. FEBS Lett. 440:361-4 (1998)); 3,4Dichloroisocoumarin (Hammed et al. Proc. Soc. Exp. Biol. Med., 219:132-7 (1998)); LEX032 (Bains et al. Eur. J. Pharmacol. 356:67-72 (1998)); N-tosyl-L-phenylalanine chloromethyl ketone (Dryjanski et al. Biochemistry 37:14151-6 (1998)); Mouse gene for the serine protease inhibitor neuroserpin (P112) (Berger et al. Gene, 214:25-33 (1998)); Rat serine protease inhibitor 2.3 gene (Paul et al. Eur. J. Biochem. 254:538-46 (1998)); Ecotin (Yang et al. J. Mol. Biol. 279:945-57 (1998)); A 14 kDa plant-related serine protease inhibitor (Roch et al. Dev. Comp. Immunol. 22(1):1-12 (1998)); Matrix-associated serine protease inhibitor TFPI-2/33 kDa MSPI (Rao et al. Int. J. Cancer 76:749-56 (1998)); ONO-3403 (Hiwasa et al. Cancer Lett. 126:221-5 (1998)); Bdellastasin (Moser et al. Eur. J. Biochem. 253:212-20 (1998)); Bikunin (Xu et al. J. Mol. Biol. 276:955-66 (1998)); Nafamostat mesilate (Meligren et al. Thromb. Haemost. 79:342-7 (1998)); The growth hormone dependent serine protease inhibitor, Spi 2.1 (Maake et al. Endocrinology 138:5630-6 (1997)); Growth factor activator inhibitor type 2, a Kunitz-type serine protease inhibitor (Kawaguchi et al. J. Biol. Chem., 272:27558-64 (1997)); Heat-stable serine protease inhibitor protein from ovaries of the desert locust, Schistocerga gregaria (Hamdaoui et al. Biochem. Biophys. Res. Commun. 238:357-60 (1997)); Human placental Hepatocyte growth factor activator inhibitor, a Kunitz-type serine protease inhibitor (Shimomura et al. J. Biol. Chem. 272:6370-6 (1997)); FUT-187, oral serine protease inhibitor (Shiozaki et al. Gan To Kaguku Ryoho, 23(14): 1971-9 (1996)); Extracellular matrix-associated serine protease inhibitors (Mr 33,000, 31,000, and 27,000 (Rao et al. (1996) Arch. Biochem. Biophys., 335:82-92); An irreversible isocoumarin serine protease inhibitor (Palencia, D. D., et al., Biol. Reprod., 55:536-42 (1996)); 4-(2-aminoethyl)-benzenesulfonyl fluoride (AEBSF) (Nakabo et al. J. Leukoc. Biol. 60:328-36 (1996)); Neuroserpin (Osterwalder, T., et al., EMBO J. 15:2944-53 (1996)); Human serine protease inhibitor alpha-1-antitrypsin (Formey et al. J. Parasitol. 82:496-502 (1996)); Rat serine protease inhibitor 2.3 (Simar-Blanchet, A. E., et al., Eur. J. Biochem., 236:638-48 (1996)); Gebaxate mesilate (Parodi, F., et al., J. Cardiothorac. Vasc. Anesth. 10:235-7 (1996)); Recombinant serine protease inhibitor, CPTI II (Stankiewicz, M., et al., (Acta Biochim. Pol., 43(3):525-9 (1996)); A cysteine-rich serine protease inhibitor (Guamerin II) (Kim, D. R., et al., J. Enzym. Inhib., 10:81-91 (1996)); Diisopropylfluorophosphate (Lundqvist, H., et al., Inflamm. Res., 44(12):510-7 (1995)); Nexin 1 (Yu, D. W., et al., J. Cell Sci., 108(Pt 12):3867-74 (1995)); LEX032 (Scalia, R., et al., Shock, 4(4):251-6 (1995)); Protease nexin I (Houenou, L. J., et al., Proc. Natl. Acad. Sci. U.S.A., 92(3):895-9 (1995)); Chymase-directed serine protease inhibitor (Woodard S. L., et al., J. Immunol., 153(11):5016-25 (1994)); N-alpha-tosyl-L-lysyl-chloromethyl ketone (TLCK) (Bourinbaiar, A. S., et al., Cell Immunol., 155(1):230-6 (1994)); Smpi56 (Ghendler, Y., et al., Exp. Parasitol., 78(2):121-31 (1994)); Schistosoma haematobium serine protease inhibitor (Blanton, R. E., et al., Mol. Biochem. Parasitol., 63(1):1-11 (1994)); Spi-1 (Warren, W. C., et al., Mol. Cell Endocrinol., 98(1):27-32 (1993)); TAME (Jessop, J. J., et al., Inflammation, 17(5):613-31 (1993)); Antithrombin III (Kalaria, R. N., et al., Am. J. Pathol., 143(3):886-93 (1993)); FOY-305 (Ohkoshi, M., et al., Anticancer Res., 13(4):963-6 (1993)); Camostat mesilate (Senda, S., et al., Intern. Med., 32(4):350-4 (1993)); Pigment epithelium-derived factor (Steele, F. R., et al., Proc. Natl. Acad. Sci. U.S.A., 90(4):1526-30 (1993)); Antistasin (Holstein, T. W., et al., FEBS Lett., 309(3):288-92 (1992)); The vaccinia virus K2L gene encodes a serine protease inhibitor (Zhou, J., et al., Virology, 189(2):678-86 (1992)); Bowman-Birk serine-protease inhibitor (Werner, M. H., et al., J. Mol. Biol., 225(3):873-89 (1992); FUT-175 (Yanamoto, H., et al., Neurosurgery, 30(3):358-63 (1992)); FUT-175; (Yanamoto, H., et al., Neurosurgery, 30(3):351-6, discussion 356-7 (1992)); PAI-I (Yreadwell, B. V., et al., J. Orthop. Res., 9(3):309-16 (1991)); 3,4-Dichloroisocoumarin (Rusbridge, N. M., et al., FEBS Lett., 268(1):133-6 (1990)); Alpha 1-antichymotrypsin (Lindmark, B. E., et al., Am. Rev. Respir. Des., 141(4 Pt 1):884-8 (1990)); P-toluenesulfonyl-L-arginine methyl ester (TAME) (Scuderi, P., J. Immunol., 143(1):168-73 (1989)); Alpha 1-antichymotrypsin (Abraham, C. R., et al., Cell, 52(4):487-501 (1988)); Contrapsin (Modha, J., et al., Parasitology, 96 (Pt 1):99-109 (1988)); Alpha 2-antiplasmin (Holmes, W. E., et al., J. Biol. Chem., 262(4):1659-64 (1987)); 3,4-dichloroisocoumarin (Harper, J. W., et al., Biochemistry, 24(8):1831-41 (1985)); Diisoprophylfluorophosphate Diisopropylfluorophosphate (Tsutsui, K., et al., Biochem. Biophys. Res. Commun., 123(1):271-7 (1984)); Gabexate mesilate (Hesse, B., et al., Pharmacol. Res. Commun., 16(7):637-45 (1984)); Phenyl methyl sulfonyl fluoride (Dufer, J., et al., Scand. J. Haematol., 32(1):25-32 (1984)); Protease inhibitor Cl-2 (McPhalen, C. A., et al., J. Mol. Biol., 168(2):445-7 (1983)); Phenylmethylsulfonyl fluoride (Sekar V., et al., Biochem. Biophys. Res. Commun., 89(2):474-8 (1979)); PGE1 (Feinstein, M. D., et al., Prostaglandine, 14(6):1075-93 (1977).

c. Combinatorial Libraries and Other Libraries

The source of compounds for the screening assays, can be libraries, including, but are not limited to, combinatorial libraries. Methods for synthesizing combinatorial libraries and characteristics of such combinatorial libraries are known in the art (See generally, Combinatorial Libraries: Synthesis, Screening and Application Potential (Cortese Ed.) Walter de Gruyter, Inc., 1995; Tietze and Lieb, Curr. Opin. Chem. Biol., 2(3):363-71 (1998); Lam, Anticancer Drug Des., 12(3):145-67 (1997); Blaney and Martin, Curr. Opin. Chem. Biol., 1(1):54-9 (1997); and Schultz and Schultz, Biotechnol. Prog., 12(6):729-43 (1996)).

Methods and strategies for generating diverse libraries, primarily peptide- and nucleotide-based oligomer libraries, have been developed using molecular biology methods and/or simultaneous chemical synthesis methodologies (see, e.g., Dower et al., Annu. Rep. Med. Chem., 26:271-280 (1991); Fodor et al., Science, 251:767-773 (1991); Jung et al., Angew. Chem. Ind. Ed. Engl., 31:367-383 (1992); Zuckerman et al., Proc. Natl. Acad. Sci. USA, 89:4505-4509 (1992); Scott et al., Science, 249:386-390 (1990); Devlin et al., Science, 249:404-406 (1990); Cwirla et al., Proc. Natl. Acad. Sci. USA, 87:6378-6382 (1990); and Gallop et al., J. Medicinal Chemistry, 37:1233-1251 (1994)). The resulting combinatorial libraries potentially contain millions of compounds that can be screened to identify compounds that exhibit a selected activity.

The libraries fall into roughly three categories: fusion-protein-displayed peptide libraries in which random peptides or proteins are presented on the surface of phage particles or proteins expressed from plasmids; support-bound synthetic chemical libraries in which individual compounds or mixtures of compounds are presented on insoluble matrices, such as resin beads (see, e.g., Lam et al., Nature, 354:82-84 (1991)) and cotton supports (see, e.g., Eichler et al., Biochemistry 32:11035-11041 (1993)); and methods in which the compounds are used in solution (see, e.g., Houghten et al., Nature, 354:84-86 (1991); Houghten et al., BioTechniques, 313:412-421 (1992); and Scott et al., Curr. Opin. Biotechnol., 5:40-48 (1994)). There are numerous examples of synthetic peptide and oligonucleotide combinatorial libraries and there are many methods for producing libraries that contain non-peptidic small organic molecules. Such libraries can be based on a basis set of monomers that are combined to form mixtures of diverse organic molecules or that can be combined to form a library based upon a selected pharmacophore monomer.

Either a random or a deterministic combinatorial library can be screened by the presently disclosed and/or claimed screening methods. In either of these two libraries, each unit of the library is isolated and/or immobilized on a solid support. In the deterministic library, one knows a priori a particular unit's location on each solid support. In a random library, the location of a particular unit is not known a priori although each site still contains a single unique unit. Many methods for preparing libraries are known to those of skill in this art (see, e.g., Geysen et al., Proc. Natl. Acad. Sci. USA, 81:3998-4002 (1984), Houghten et al., Proc. Natl. Acad. Sci. USA, 81:5131-5135 (1985)). Combinatorial libraries generated by any techniques known to those of skill in the art are contemplated (see, e.g., Table 1 of Schultz and Schultz, Biotechnol. Prog., 12(6):729-43 (1996)) for screening; Bartel et al., Science, 261:1411-1418 (1993); Baumbach et al. BioPharm, (Can):24-35 (1992); Bock et al. Nature, 355:564-566 (1992); Borman, S., Combinatorial chemists focus on small molecules molecular recognition, and automation, Chem. Eng. News, 2(12):29 (1996); Boublik, et al., Eukaryotic Virus Display: Engineering the Major Surface Glycoproteins of the Autographa California Nuclear Polyhedrosis Virus (ACNPV) for the Presentation of Foreign Proteins on the Virus Surface, Bio/Technology, 13:1079-1084 (1995); Brenner, et al., Encoded Combinatorial Chemistry, Proc. Natl. Acad Sci. U.S.A., 89:5381-5383 (1992); Caflisch, et al., Computational Combinatorial Chemistry for De Novo Ligand Design: Review and Assessment, Perspect. Drug Discovery Des., 3:51-84 (1995); Cheng, et al., Sequence-Selective Peptide Binding with a Peptido-A,B-trans-steroidal Receptor Selected from an Encoded Combinatorial Library, J. Am. Chem. Soc., 118:1813-1814 (1996); Chu, et al., Affinity Capillary Electrophoresis to Identify the Peptide in A Peptide Library that Binds Most Tightly to Vancomycin, J. Org. Chem., 58:648-652 (1993); Clackson, et al., Making Antibody Fragments Using Phage Display Libraries, Nature, 352:624-628 (1991); Combs, et al., Protein Structure-Based Combinatorial Chemistry: Discovery of Non-Peptide Binding Elements to Src SH3 Domain, J. Am. Chem. Soc., 118:287-288 (1996); Cwirla, et al., Peptides On Phage: A Vast Library of Peptides for Identifying Ligands, Proc. Natl. Acad. Sci. U.S., 87:6378-6382 (1990); Ecker, et al., Combinatorial Drug Discovery: Which Method will Produce the Greatest Value, Bio/Technology, 13:351-360 (1995); Ellington, et al., In Vitro Selection of RNA Molecules That Bind Specific Ligands, Nature, 346:818-822 (1990); Ellman, J. A., Variants of Benzodiazephines, J. Am. Chem. Soc., 114:10997 (1992); Erickson, et al., The Proteins; Neurath, H., Hill, R. L., Eds.: Academic: New York, 1976; pp. 255-257; Felici, et al., J. Mol. Biol., 222:301-310 (1991); Fodor, et al., Light-Directed, Spatially Addressable Parallel Chemical Synthesis, Science, 251:767-773 (1991); Francisco, et al., Transport and Anchoring of Beta-Lactamase to the External Surface of E. Coli., Proc. Natl. Acad. Sci. U.S.A., 89:2713-2717 (1992); Georgiou, et al., Practical Applications of Engineering Gram-Negative Bacterial Cell Surfaces, TIBTECH, 11:6-10 (1993); Geysen, et al., Use of peptide synthesis to probe viral antigens for epitopes to a resolution of a single amino acid, Proc. Natl. Acad. Sci. U.S.A., 81:3998-4002 (1984); Glaser, et al., Antibody Engineering by Codon-Based Mutagenesis in a Filamentous Phage Vector System, J. Immunol., 149:3903-3913 (1992); Gram, et al., In vitro selection and affinity maturation of antibodies from a naive combinatorial immunoglobulin library, Proc. Natl. Acad. Sci., 89:3576-3580 (1992); Han, et al., Liquid-Phase Combinatorial Synthesis, Proc. Natl. Acad. Sci. U.S.A., 92:6419-6423 (1995); Hoogenboom, et al., Multi-Subunit Proteins on the Surface of Filamentous Phage: Methodologies for Displaying Antibody (Fab) Heavy and Light Chains, Nucleic Acids Res., 19:4133-4137 (1991); Houghten, et al., General Method for the Rapid Solid-Phase Synthesis of Large Numbers of Peptides: Specificity of Antigen-Antibody Interaction at the Level of Individual Amino Acids, Proc. Natl. Acad. Sci. U.S.A., 82:5131-5135 (1985); Houghten, et al., The Use of Synthetic Peptide Combinatorial Libraries for the Determination of Peptide Ligands in Radio-Receptor Assays-Opiod-Peptides, Bioorg. Med. Chem. Lett., 3:405-412 (1993); Houghten, et al., Generation and Use of Synthetic Peptide Combinatorial Libraries for Basic Research and Drug Discovery, Nature, 354:84-86 (1991); Huang, et al., Discovery of New Ligand Binding Pathways in Myoglobin by Random Mutagenesis, Nature Struct. Biol., 1:226-229 (1994); Huse, et al., Generation of a Large Combinatorial Library of the Immunoglobulin Repertoire In Phage Lambda, Science, 246:1275-1281 (1989); Janda, K. D., New Strategies for the Design of Catalytic Antibodies, Biotechnol. Prog., 6:178-181 (1990); Jung, et al., Multiple Peptide Synthesis Methods and Their Applications, Angew. Chem. Int. Ed. Engl., 31:367-486 (1992); Kang, et al., Linkage of Recognition and Replication Functions By Assembling Combinatorial Antibody Fab Libraries Along Phage Surfaces, Proc. Natl. Acad. Sci. U.S.A., 88:4363-4366 (1991a); Kang, et al., Antibody Redesign by Chain Shuffling from Random Combinatorial Immunoglobulin Libraries, Proc. Natl. Acad. Sci. U.S.A., 88:11120-11123 (1991b); Kay, et al., An M13 Phage Library Displaying Random 38-Amino-Acid-Peptides as a Source of Novel Sequences with Affinity to Selected Targets Genes, Gene, 128:59-65 (1993); Lam, et al., A new type of synthetic peptide library for identifying ligand-binding activity, Nature, 354:82-84 (1991) (published errata appear in Nature, 358:434 (1992) and Nature, 360:768 (1992); Lebl, et al., One Bead One Structure Combinatorial Libraries, Biopolymers (Pept. Sci.), 37:177-198 (1995); Lerner, et al., Antibodies without Immunization, Science, 258:1313-1314 (1992); Li, et al., Minimization of a Polypeptide Hormone, Science, 270:1657-1660 (1995); Light, et al., Display of Dimeric Bacterial Alkaline Phosphatase on the Major Coat Protein of Filamentous Bacteriophage, Bioorg. Med. Chem. Lett., 3:1073-1079 (1992); Little, et al., Bacterial Surface Presentation of Proteins and Peptides: An Alternative to Phage Technology, Trends Biotechnol., 11:3-5 (1993); Marks, et al., By-Passing Immunization. Human Antibodies from V-Gene Libraries Displayed on Phage, J. Mol. Biol., 222:581-597 (1991); Matthews, et al., Substrate Phage: Selection of Protease Substrates by Monovalent Phage Display, Science, 260:1113-1117 (1993); McCafferty, et al., Phage Enzymes: Expression and Affinity Chromatography of Functional Alkaline Phosphatase on the Surface of Bacteriophage, Protein Eng., 4:955-961 (1991); Menger, et al., Phosphatase Catalysis Developed Via Combinatorial Organic Chemistry, J. Org. Chem., 60:6666-6667 (1995); Nicolaou, et al., Angew. Chem. Int. Ed. Engl., 34:2289-2291 (1995); Oldenburg, et al., Peptide Ligands for A Sugar-Binding Protein Isolated from a Random Peptide Library, Proc. Natl. Acad. Sci. U.S.A., 89:5393-5397 (1992); Parmley, et al., Antibody-Selectable Filamentous fd Phage Vectors: Affinity Purification of Target Genes, Genes, 73:305-318 (1988); Pinilla, et al., Synthetic Peptide Combinatorial Libraries (SPCLS)—Identification of the Antigenic Determinant of Beta-Endorphin Recognized by Monoclonal Antibody-3E7, Gene, 128:71-76 (1993); Pinilla, et al., Review of the Utility of Soluble Combinatorial Libraries, Biopolymers, 37:221-240 (1995); Pistor, et al., Expression of Viral Hemagglutinin On the Surface of E. Coli., Klin. Wochenschr., 66:110-116 (1989); Pollack, et al., Selective Chemical Catalysis by an Antibody, Science, 234:1570-1572 (1986); Rigler, et al., Fluorescence Correlations, Single Molecule Detection and Large Number Screening: Applications in Biotechnology, J. Biotechnol., 41:177-186 (1995); Sarvetnick, et al., Increasing the Chemical Potential of the Germ-Line Antibody Repertoire, Proc. Natl. Acad. Sci. U.S.A., 90:4008-4011 (1993); Sastry et al., Cloning of the Immunological Repertoire in Escherichia Coli for Generation of Monoclonal Catalytic Antibodies: Construction of a Heavy Chain Variable Region-Specific cDNA Library, Proc. Natl. Acad. Sci. U.S.A., 86:5728-5732 (1989); Scott, et al., Searching for Peptide Ligands with an Epitope Library, Science, 249:386-390 (1990); Sears, et al., Engineering Enzymes for Bioorganic Synthesis: Peptide Bond Formation, Biotechnol. Prog., 12:423-433 (1996); Simon, et. al., Peptides: A Modular Approach to Drug Discovery, Proc. Natl. Acad. Sci. U.S.A., 89:9367-9371 (1992); Still, et al., Discovery of Sequence-Selective Peptide Binding by Synthetic Receptors Using Encoded Combinatorial Libraries, Acc. Chem. Res., 29:155-163 (1996); Thompson, et al., Synthesis and Applications of Small Molecule Libraries, Chem. Rev., 96:555-600 (1996); Tramontano, et al., Catalytic Antibodies, Science, 234:1566-1570 (1986); Wrighton, et al., Small Peptides as Potent Mimetics of the Protein Hormone Erythropoietin, Science, 273:458-464 (1996); York, et al., Combinatorial mutagenesis of the reactive site region in plasminogen activator inhibitor I, J. Biol. Chem., 266:8595-8600 (1991); Zebedee, et al., Human Combinatorial Antibody Libraries to Hepatitis B Surface Antigen, Proc. Natl. Acad. Sci. U.S.A., 89:3175-3179 (1992); Zuckerman, et al., Identification of Highest-Affinity Ligands by Affinity Selection from Equimolar Peptide Mixtures Generated by Robotic Synthesis, Proc. Natl. Acad. Sci. U.S.A., 89:4505-4509 (1992).

For example, peptides that bind to an MTSP20 polypeptide or a protease domain of an SP protein can be identified using phage display libraries. In an exemplary embodiment, this method can include (a) contacting phage from a phage library with the MTSP20 polypeptide or a protease domain thereof; (b) isolating phage that bind to the protein; and (c) determining the identity of at least one peptide coded by the isolated phage to identify a peptide that binds to an MTSP20 polypeptide.

H. Modulators of an Activity of MTSP20 Polypeptides

Provided herein are compounds, identified by screening or produced using an MTSP20 polypeptide or protease domains thereof in other screening methods, that modulate an activity of an MTSP20. These compounds act by directly interacting with the MTSP20 polypeptide or by altering transcription or translation thereof. Such molecules include, but are not limited to, antibodies that specifically react with an MTSP20 polypeptide, particularly with a protease domain thereof, antisense nucleic acids or double-stranded RNA (dsRNA) such as RNAi, that alter expression of an MTSP20 polypeptide, antibodies, peptide mimetics and other such compounds.

1. Antibodies

Antibodies, including polyclonal and monoclonal antibodies, that specifically bind to the MTSP20 polypeptide provided herein, particularly to the single chain protease domains thereof or the activated forms of the full-length or protease domain or the zymogen form, are provided.

Generally, the antibody is a monoclonal antibody, and typically the antibody specifically binds to a protease domain of the MTSP20 polypeptide. In particular embodiments, antibodies to each of the single chain and or any of the multi-chain forms of the protease domains of MTSP20 are provided. Also provided are antibodies that specifically bind to any domain of MTSP20 and to multi-chain forms thereof.

The MTSP20 polypeptide and domains, fragments, homologs and derivatives thereof can be used as immunogens to generate antibodies that specifically bind such immunogens. Such antibodies include but are not limited to polyclonal, monoclonal, chimeric, single chain, Fab fragments, and an Fab expression library. In a specific embodiment, antibodies to human MTSP20 polypeptide are produced. In another embodiment, complexes formed from fragments of an MTSP20 polypeptide, that contain the serine protease domain are used as immunogens for antibody production.

Various procedures known in the art can be used for the production of polyclonal antibodies to MTSP20 polypeptide, its domains, derivatives, fragments or analogs. For production of the antibody, various host animals can be immunized by injection with the native MTSP20 polypeptide or a synthetic version, or a derivative of the foregoing, such as a cross-linked MTSP20 polypeptide. Such host animals include but are not limited to rabbits, mice, rats, etc. Various adjuvants can be used to increase the immunological response, depending on the host species, and include but are not limited to Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacille Calmette-Guerin (BCG) and corynebacterium parvum.

For preparation of monoclonal antibodies directed towards an MTSP20 polypeptide or domains, derivatives, fragments or analogs thereof, any technique that provides for the production of antibody molecules by continuous cell lines in culture can be used. Such techniques include but are not restricted to the hybridoma technique originally developed by Kohler and Milstein (Nature 256:495-497 (1975)), the trioma technique, the human B-cell hybridoma technique (Kozbor et al., Immunology Today 4:72 (1983)), and the EBV hybridoma technique to produce human monoclonal antibodies (Cole et al., in Monoclonal Antibodies and Cancer Therapy, Alan R. Liss, Inc., pp. 77-96 (1985)). In an additional embodiment, monoclonal antibodies can be produced in germ-free animals utilizing recent technology (PCT/US90/02545). Human antibodies can be used and can be obtained by using human hybridomas (Cote et al., Proc. Natl. Acad. Sci. USA 80:2026-2030 (1983)), or by transforming human B cells with EBV virus in vitro (Cole et al., in Monoclonal Antibodies and Cancer Therapy, Alan R. Liss, Inc., pp. 77-96 (1985)). Techniques developed for the production of “chimeric antibodies” (Morrison et al., Proc. Natl. Acad. Sci. USA 81:6851-6855 (1984); Neuberger et al., Nature 312:604-608 (1984); Takeda et al., Nature 314:452-454 (1985)) by splicing the genes from a mouse antibody molecule specific for the MTSP20 polypeptide together with genes from a human antibody molecule of appropriate biological activity can be used.

MTSP20-encoding nucleic acid molecules or portions thereof can be used in DNA immunization protocols to produce antibodies that bind to MTSP20 (see, e.g., U.S. Pat. No. 5,795,872 and U.S. Pat. No. 5,643,578 and U.S. Pat. No. 6,337,072).

Techniques described for the production of single chain antibodies (U.S. Pat. No. 4,946,778) can be adapted to produce MTSP20 polypeptide-specific single chain antibodies. An additional embodiment uses the techniques described for the construction of Fab expression libraries (Huse et al., Science 246:1275-1281 (1989)) to allow rapid and easy identification of monoclonal Fab fragments with the desired specificity for MTSP20 polypeptide or domains, derivatives, or analogs thereof. Non-human antibodies can be “humanized” by known methods (see, e.g., U.S. Pat. No. 5,225,539).

Antibody fragments that specifically bind to MTSP20 polypeptide or epitopes thereof can be generated by techniques known in the art. For example, such fragments include but are not limited to: the F(ab′)₂ fragment, which can be produced by pepsin digestion of the antibody molecule; the Fab′ fragments that can be generated by reducing the disulfide bridges of the F(ab′)2 fragment, the Fab fragments that can be generated by treating the antibody molecule with papain and a reducing agent, and Fv fragments.

In the production of antibodies, screening for the desired antibody can be accomplished by techniques known in the art, e.g., ELISA (enzyme-linked immunosorbent assay). To select antibodies specific for a particular domain of the MTSP20 polypeptide one can assay generated hybridomas for a product that binds to the fragment of the MTSP20 polypeptide that contains such a domain.

The foregoing antibodies can be used in methods known in the art relating to the localization and/or quantitation of MTSP20 polypeptide proteins, e.g., for imaging these proteins, measuring levels thereof in appropriate physiological samples, in, for example, diagnostic methods. In another embodiment, anti-MTSP20 polypeptide antibodies, or fragments thereof, containing the binding domain are used as therapeutic agents.

2. Peptides, Polypeptides and Peptide Mimetics

Provided herein are methods for identifying molecules that bind to and modulate an activity of MTSP20 polypeptides. Included among molecules that bind to MTSPs, such as a single chain protease domain or catalytically active fragments thereof, are peptides, polypeptides and peptide mimetics, including cyclic peptides. Peptide mimetics are molecules or compounds that mimic the necessary molecular conformation of a ligand or polypeptide for specific binding to a target molecule such as an MTSP20 polypeptide. In an exemplary embodiment, the peptides, polypeptides and peptide mimetics bind to a protease domain of an MTSP20 polypeptide. Such peptides and peptide mimetics include those of antibodies that specifically bind to an MTSP20 polypeptide and, typically, bind to a protease domain of an MTSP20 polypeptide. The peptides, polypeptides and peptide mimetics identified by methods provided herein can be agonists or antagonists of MTSP20 polypeptides.

Such peptides, polypeptides and peptide mimetics are useful for diagnosing, treating, preventing, and screening for a disease or disorder associated with MTSP20 polypeptide activity in a mammal. In addition, the peptides and peptide mimetics are useful for identifying, isolating, and purifying molecules or compounds that modulate an activity of an MTSP20 polypeptide, or specifically bind to an MTSP20 polypeptide, generally a protease domain of an MTSP20 polypeptide. Low molecular weight peptides and peptide mimetics can have strong binding properties to a target molecule, e.g., an MTSP20 polypeptide or a protease domain of an MTSP20 polypeptide.

Peptides, polypeptides and peptide mimetics that bind to MTSP20 polypeptides as described herein can be administered to mammals, including humans, to modulate MTSP20 polypeptide activity. Thus, methods for therapeutic treatment and prevention of neoplastic diseases comprise administering a peptide, polypeptide or peptide mimetic compound in an amount sufficient to modulate such activity are provided. Thus, also provided herein are methods for treating a subject having such a disease or disorder in which a peptide, polypeptide or peptide mimetic compound is administered to the subject in a therapeutically effective dose or amount.

Compositions containing the peptides, polypeptides or peptide mimetics can be administered for prophylactic and/or therapeutic treatments. In therapeutic applications, compositions can be administered to a patient already suffering from a disease, as described above, in an amount sufficient to cure or at least partially arrest the symptoms of the disease and its complications. Amounts effective for this use will depend on the severity of the disease and the weight and general state of the patient and can be empirically determined.

In prophylactic applications, compositions containing the peptides, polypeptides and peptide mimetics are administered to a patient susceptible to or otherwise at risk of a particular disease. Such an amount is defined to be a “prophylactically effective dose.” In this use, the precise amounts again depend on the patient's state of health and weight. Accordingly, the peptides, polypeptides and peptide mimetics that bind to an MTSP20 polypeptide can be used to prepare pharmaceutical compositions containing, as an active ingredient, at least one of the peptides, polypeptides or peptide mimetics in association with a pharmaceutical carrier or diluent. The compounds can be administered, for example, by oral, pulmonary, parenteral (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

Peptides, polypeptides and peptide mimetics that bind to MTSP20 polypeptides are useful in vitro as unique tools for understanding the biological role of MTSP20 polypeptides, including the evaluation of the many factors thought to influence, and be influenced by, the production of MTSP20 polypeptide. Such peptides, polypeptides and peptide mimetics are also useful in the development of other compounds that bind to and modulate an activity of an MTSP20 polypeptide, because such compounds provide important information on the relationship between structure and activity that should facilitate such development.

The peptides, polypeptides and peptide mimetics are also useful as competitive binders in assays to screen for new MTSP20 polypeptides or MTSP20 polypeptide agonists. In such assay embodiments, the compounds can be used without modification or can be modified in a variety of ways; for example, by labeling, such as covalently or non-covalently joining a moiety which directly or indirectly provides a detectable signal. In any of these assays, the materials thereto can be labeled either directly or indirectly. Possibilities for direct labeling include label groups such as: radiolabels such as ¹²⁵I enzymes (U.S. Pat. No. 3,645,090), peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups. The compounds can also include spacers or linkers in cases where the compounds are to be attached to a solid support.

Moreover, based on their ability to bind to an MTSP20 polypeptide, the peptides, polypeptides and peptide mimetics can be used as reagents for detecting MTSP20 polypeptides in living cells, fixed cells, in biological fluids, in tissue homogenates and in purified, natural biological materials. For example, by labelling such peptides, polypeptides and peptide mimetics, cells having MTSP20 polypeptides can be identified. In addition, based on their ability to bind an MTSP20 polypeptide, the peptides, polypeptides and peptide mimetics can be used in in situ staining, FACS (fluorescence-activated cell sorting), Western blotting, ELISA and other analytical protocols. Based on their ability to bind to an MTSP20 polypeptide, the peptides, polypeptides and peptide mimetics can be used in purification of MTSP20 polypeptides or in purifying cells expressing the MTSP20 polypeptides, e.g., a polypeptide encoding a protease domain of an MTSP20 polypeptide.

The peptides, polypeptides and peptide mimetics can also be used as commercial reagents for various medical research and diagnostic uses. The activity of the peptides and peptide mimetics can be evaluated either in vitro or in vivo in one of the numerous models described in McDonald (1992) Am. J. of Pediatric Hematology/Oncology, 14:8-21.

3. Peptide, Polypeptide and Peptide Mimetic Therapy

Peptide analogs are commonly used in the pharmaceutical industry as non-peptide drugs with properties analogous to those of the template peptide. These types of non-peptide compounds are termed “peptide mimetics” or “peptidomimetics” (Luthman et al., A Textbook of Drug Design and Development, 14:386-406, 2nd Ed., Harwood Academic Publishers (1996); Joachim Grante (1994) Angew. Chem. Int. Ed. Engl., 33:1699-1720; Fauchere (1986) J. Adv. Drug Res., 15:29; Veber and Freidinger (1985) TINS, p. 392; and Evans et al. (1987) J. Med. Chem. 30:1229). Peptide mimetics that are structurally similar to therapeutically useful peptides can be used to produce an equivalent or enhanced therapeutic or prophylactic effect. Preparation of peptidomimetics and structures thereof are known to those of skill in this art.

Systematic substitution of one or more amino acids of a consensus sequence with a D-amino acid of the same type (e.g., D-lysine in place of L-lysine) can be used to generate more stable peptides. In addition, constrained peptides containing a consensus sequence or a substantially identical consensus sequence variation can be generated by methods known in the art (Rizo et al. (1992) An. Rev. Biochem., 61:387, incorporated herein by reference); for example, by adding internal cysteine residues capable of forming intramolecular disulfide bridges which cyclize the peptide.

Those skilled in the art appreciate that modifications can be made to the peptides and mimetics without deleteriously effecting the biological or functional activity of the peptide. Further, the skilled artisan would know how to design non-peptide structures in three dimensional terms, that mimic the peptides that bind to a target molecule, e.g., an MTSP20 polypeptide or, generally, a protease domain of MTSP20 polypeptides (see, e.g., Eck and Sprang (1989) J. Biol. Chem., 26: 17605-18795).

When used for diagnostic purposes, the peptides and peptide mimetics can be labeled with a detectable label and, accordingly, the peptides and peptide mimetics without such a label can serve as intermediates in the preparation of labeled peptides and peptide mimetics. Detectable labels can be molecules or compounds, which when covalently attached to the peptides and peptide mimetics, permit detection of the peptide and peptide mimetics in vivo, for example, in a patient to whom the peptide or peptide mimetic has been administered, or in vitro, e.g., in a sample or cells. Suitable detectable labels are well known in the art and include, by way of example, radioisotopes, fluorescent labels (e.g., fluorescein), and others. The particular detectable label employed is not critical and is selected to be detectable at non-toxic levels. Selection of such labels is well within the skill of the art.

Covalent attachment of a detectable label to the peptide or peptide mimetic is accomplished by conventional methods well known in the art. For example, when the ¹²⁵I radioisotope is employed as the detectable label, covalent attachment of ¹²⁵I to the peptide or the peptide mimetic can be achieved by incorporating the amino acid tyrosine into the peptide or peptide mimetic and then iodinating the peptide (see, e.g., Weaner et al. (1994) Synthesis and Applications of Isotopically Labelled Compounds, pp. 137-140). If tyrosine is not present in the peptide or peptide mimetic, incorporation of tyrosine to the N or C terminus of the peptide or peptide mimetic can be achieved by well known chemistry. Likewise, ³²p can be incorporated onto the peptide or peptide mimetic as a phosphate moiety through, for example, a hydroxyl group on the peptide or peptide mimetic using conventional chemistry.

Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Peptides, polypeptides and peptide mimetics that can bind to an MTSP20 polypeptide or a protease domain of MTSP20 polypeptides and/or modulate an activity thereof, or exhibit an MTSP20 polypeptide activity, can be used for treatment of neoplastic disease. The peptides, polypeptides and peptide mimetics can be delivered, in vivo or ex vivo, to the cells of a subject in need of treatment. Further, peptides which have MTSP20 polypeptide activity can be delivered, in vivo or ex vivo, to cells which carry mutant or missing alleles encoding the MTSP20 polypeptide gene. Any of the techniques described herein or known to the skilled artisan can be used for preparation and in vivo or ex vivo delivery of such peptides, polypeptides and peptide mimetics that are substantially free of other human proteins. For example, the peptides, polypeptides and peptide mimetics can be readily prepared by expression in a microorganism or synthesis in vitro.

The peptides or peptide mimetics can be introduced into cells, in vivo or ex vivo, by microinjection or by use of liposomes, for example. Alternatively, the peptides, polypeptides or peptide mimetics can be taken up by cells, in vivo or ex vivo, actively or by diffusion. In addition, extracellular application of the peptide, polypeptide or peptide mimetic can be sufficient to effect treatment of a neoplastic disease. Other molecules, such as drugs or organic compounds, that: 1) bind to a MTSP20 polypeptide or protease domain thereof; or 2) have a similar function or activity to an MTSP20 polypeptide or protease domain thereof, can be used in methods for treatment.

4. Rational Drug Design

The goal of rational drug design is to produce structural analogs of biologically active polypeptides or peptides of interest or of small molecules or peptide mimetics with which they interact (e.g., agonists and antagonists) in order to fashion drugs which are, e.g., more active or stable forms thereof; or which, for example, enhance or interfere with the function of a polypeptide in vivo (e.g., an MTSP20 polypeptide). In one approach, one first determines the three-dimensional structure of a protein of interest (e.g., an MTSP20 polypeptide or polypeptide having a protease domain) or, for example, of an MTSP20 polypeptide-ligand complex, by X-ray crystallography, by computer modeling or most typically, by a combination of approaches (see, e.g., Erickson et al. 1990). Also, useful information regarding the structure of a polypeptide can be gained by modeling based on the structure of homologous proteins. In addition, peptides can be analyzed by an alanine scan. In this technique, an amino acid residue is replaced by Ala, and its effect on the peptide's activity is determined. Each of the amino acid residues of the peptide is analyzed in this manner to determine the important regions of the peptide.

Also, a polypeptide or peptide that binds to an MTSP20 polypeptide or, generally, a protease domain of an MTSP20 polypeptide, can be selected by a functional assay, and then the crystal structure of this polypeptide or peptide can be determined. The polypeptide can be, for example, an antibody specific for an MTSP20 polypeptide or the protein domain of an MTSP20 polypeptide. This approach can yield a pharmacophore upon which subsequent drug design can be based. Further, it is possible to bypass the crystallography altogether by generating anti-idiotypic polypeptides or peptides, (anti-ids) to a functional, pharmacologically active polypeptide or peptide that binds to an MTSP20 polypeptide or protease domain of an MTSP20 polypeptide. As a mirror image of a mirror image, the binding site of the anti-ids is expected to be an analog of the original target molecule, e.g., an MTSP20 polypeptide or polypeptide having an MTSP20 polypeptide. The anti-id could then be used to identify and isolate peptides from banks of chemically or biologically produced banks of peptides. Selected peptides would then act as the pharmacophore.

Thus, one can design drugs which have, for example, improved activity or stability or which act as modulators (e.g., inhibitors, agonists or antagonists) of MTSP20 polypeptide activity, and are useful in the methods, particularly the methods for diagnosis, treatment, prevention, and screening of a neoplastic disease. By virtue of the availability of nucleic acid that encodes MTSP20 polypeptides, sufficient amounts of the MTSP20 polypeptide can be made available to perform such analytical studies as X-ray crystallography. In addition, the knowledge of the amino acid sequence of an MTSP20 polypeptide or a protease domain thereof, e.g., a protease domain encoded by the amino acid sequence of SEQ ID Nos. 16 and 6, can provide guidance on computer modeling techniques in place of, or in addition to, X-ray crystallography.

Methods of Identifying Peptides and Peptide Mimetics that Bind to MTSP20 Polypeptides

Peptides having a binding affinity to an MTSP20 polypeptide provided herein (e.g., an MTSP20 polypeptide or a polypeptide having a protease domain of an MTSP20 polypeptide) can be readily identified, for example, by random peptide diversity generating systems coupled with an affinity enrichment process. Specifically, random peptide diversity generating systems include the “peptides on plasmids” system (see, e.g., U.S. Pat. Nos. 5,270,170 and 5,338,665); the “peptides on phage” system (see, e.g., U.S. Pat. No. 6,121,238 and Cwirla, et al. (1990) Proc. Natl. Acad. Sci. U.S.A. 87:6378-6382); the “polysome system;” the “encoded synthetic library (ESL)” system; and the “very large scale immobilized polymer synthesis” system (see, e.g., U.S. Pat. No. 6,121,238; and Dower et al. (1991) An. Rep. Med. Chem. 26:271-280).

For example, using the procedures described above, random peptides can generally be designed to have a defined number of amino acid residues in length (e.g., 12). To generate the collection of oligonucleotides encoding the random peptides, the codon motif (NNK)x, where N is nucleotide A, C, G, or T (equimolar; depending on the methodology employed, other nucleotides can be employed), K is G or T (equimolar), and x is an integer corresponding to the number of amino acids in the peptide (e.g., 12) can be used to specify any one of the 32 possible codons resulting from the NNK motif: 1 for each of 12 amino acids, 2 for each of 5 amino acids, 3 for each of 3 amino acids, and only one of the three stop codons. Thus, the NNK motif encodes all of the amino acids, encodes only one stop codon, and reduces codon bias.

The random peptides can be presented, for example, either on the surface of a phage particle, as part of a fusion protein containing either the pill or the pVIII coat protein of a phage fd derivative (peptides on phage) or as a fusion protein with the LacI peptide fusion protein bound to a plasmid (peptides on plasmids). The phage or plasmids, including the DNA encoding the peptides, can be identified and isolated by an affinity enrichment process using immobilized MTSP20 polypeptide having a protease domain. The affinity enrichment process, sometimes called “panning,” typically involves multiple rounds of incubating the phage, plasmids, or polysomes with the immobilized MTSP20 polypeptide, collecting the phage, plasmids, or polysomes that bind to the MTSP20 polypeptide (along with the accompanying DNA or mRNA), and producing more of the phage or plasmids (along with the accompanying LacI-peptide fusion protein) collected.

Characteristics of Peptides and Peptide Mimetics

Among the peptides, polypeptides and peptide mimetics for therapeutic application are those of having molecular weights from about 250 to about 8,000 daltons. If such peptides are oligomerized, dimerized and/or derivatized with a hydrophilic polymer (e.g., to increase the affinity and/or activity of the compounds), the molecular weights of such peptides can be substantially greater and can range anywhere from about 500 to about 120,000 daltons, generally from about 8,000 to about 80,000 daltons. Such peptides can contain 9 or more amino acids that are naturally occurring or synthetic (non-naturally occurring) amino acids. One skilled in the art can determine the affinity and molecular weight of the peptides and peptide mimetics suitable for therapeutic and/or diagnostic purposes (e.g., see Dower et al., U.S. Pat. No. 6,121,238).

The peptides can be covalently attached to one or more of a variety of hydrophilic polymers. Suitable hydrophilic polymers include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives. When the peptide compounds are derivatized with such polymers, their solubility and circulation half-lives can be increased with little, if any, diminishment in their binding activity. The peptide compounds can be dimerized and each of the dimeric subunits can be covalently attached to a hydrophilic polymer. The peptide compounds can be PEGylated, i.e., covalently attached to polyethylene glycol (PEG).

5. Methods of Preparing Peptides and Peptide Mimetics

Peptides that bind to MTSP20 polypeptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology (see, e.g., Merrifield (1963) J. Am. Chem. Soc., 85:2149, incorporated herein by reference.)

Using the “encoded synthetic library” or “very large scale immobilized polymer synthesis” systems (see, e.g., U.S. Pat. No. 5,925,525, and 5,902,723), the minimum size of a peptide with an activity of interest can be determined. In addition all peptides that form the group of peptides that differ from the desired motif (or the minimum size of that motif) in one, two, or more residues can be prepared. This collection of peptides then can be screened for the ability to bind to the target molecule, e.g., MTSP20 polypeptide or, generally, a protease domain of an MTSP20 polypeptide. This immobilized polymer synthesis system or other peptide synthesis methods can also be used to synthesize truncation analogs and deletion analogs and combinations of truncation and deletion analogs of the peptide compounds.

These procedures can also be used to synthesize peptides in which amino acids other than the 20 naturally occurring, genetically encoded amino acids are substituted at one, two, or more positions of the peptide. For instance, naphthylalanine can be substituted for tryptophan, facilitating synthesis. Other synthetic amino acids that can be substituted into the peptides include L-hydroxypropyl, L-3,4-dihydroxy-phenylalanyl, d amino acids such as L-d-hydroxylysyl and D-d-methylalanyl, L-a-methylalanyl, β amino acids, and isoquinolyl. D amino acids and non-naturally occurring synthetic amino acids can also be incorporated into the peptides (see, e.g., Roberts et al. (1983) Unusual Amino/Acids in Peptide Synthesis, 5(6):341-449).

The peptides also can be modified by phosphorylation (see, e.g., W. Bannwarth et al. (1996) Bioorganic and Medicinal Chemistry Letters, 6(17):2141-2146), and other methods for making peptide derivatives (see, e.g., Hruby et al. (1990) Biochem. J., 268(2):249-262). Thus, peptide compounds also serve as a basis to prepare peptide mimetics with similar or improved biological activity.

Those of skill in the art recognize that a variety of techniques are available for constructing peptide mimetics with the same or similar desired biological activity as the corresponding peptide compound but with more favorable activity than the peptide with respect to solubility, stability, and susceptibility to hydrolysis and proteolysis (see, e.g., Morgan et al. (1989) An. Rep. Med. Chem., 24:243-252). Methods for preparing peptide mimetics modified at the N-terminal amino group, the C-terminal carboxyl group, and/or changing one or more of the amido linkages in the peptide to a non-amido linkage are known to those of skill in the art.

Amino terminus modifications include, but are not limited to, alkylating, acetylating and adding a carbobenzoyl group, forming a succinimide group (see, e.g., Murray et al. (1995) Burger's Medicinal Chemistry and Drug Discovery, 5th ed., Vol. 1, Manfred E. Wolf, ed., John Wiley and Sons, Inc.). C-terminal modifications include mimetics wherein the C-terminal carboxyl group is replaced by an ester, an amide or modifications to form a cyclic peptide.

In addition to N-terminal and C-terminal modifications, the peptide compounds, including peptide mimetics, can advantageously be modified with or covalently coupled to one or more of a variety of hydrophilic polymers. It has been found that when peptide compounds are derivatized with a hydrophilic polymer, their solubility and circulation half-lives can be increased and their immunogenicity is masked, with little, if any, diminishment in their binding activity. Suitable nonproteinaceous polymers include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives. Generally, such hydrophilic polymers have an average molecular weight ranging from about 500 to about 100,000 daltons, including from about 2,000 to about 40,000 daltons and, from about 5,000 to about 20,000 daltons. The hydrophilic polymers also can have an average molecular weights of about 5,000 daltons, 10,000 daltons and 20,000 daltons.

Methods for derivatizing peptide compounds or for coupling peptides to such polymers have been described (see, e.g., Zallipsky (1995) Bioconjugate Chem., 6:150-165; Monfardini et al. (1995) Bioconjugate Chem., 6:62-69; U.S. Pat. No. 4,640,835; U.S. Pat. No. 4,496,689; U.S. Pat. No. 4,301,144; U.S. Pat. No. 4,670,417; U.S. Pat. No. 4,791,192; U.S. Pat. No. 4,179,337 and WO 95/34326, all of which are incorporated by reference in their entirety herein).

Other methods for making peptide derivatives are described, for example, in Hruby et al. (1990), Biochem J., 268(2):249-262, which is incorporated herein by reference. Thus, the peptide compounds also serve as structural models for non-peptidic compounds with similar biological activity. Those of skill in the art recognize that a variety of techniques are available for constructing compounds with the same or similar desired biological activity as a particular peptide compound but with more favorable activity with respect to solubility, stability, and susceptibility to hydrolysis and proteolysis (see, e.g., Morgan et al. (1989) An. Rep. Med. Chem., 24:243-252, incorporated herein by reference). These techniques include replacing the peptide backbone with a backbone composed of phosphonates, amidates, carbamates, sulfonamides, secondary amines, and N-methylamino acids.

Peptide compounds can exist in a cyclized form with an intramolecular disulfide bond between the thiol groups of the cysteines. Alternatively, an intermolecular disulfide bond between the thiol groups of the cysteines can be produced to yield a dimeric (or higher oligomeric) compound. One or more of the cysteine residues can also be substituted with a homocysteine.

I. Conjugates

A conjugate, containing: a) a single chain protease domain (or proteolytically active portion thereof) or two or a plurality of protease domains of an MTSP20 polypeptide or a full length zymogen, activated form thereof, or two or single chain protease domain thereof; and b) a targeting agent linked to the MTSP20 polypeptide directly or via a linker, wherein the agent facilitates: i) affinity isolation or purification of the conjugate; ii) attachment of the conjugate to a surface; iii) detection of the conjugate; or iv) targeted delivery to a selected tissue or cell, is provided herein. The conjugate can be a chemical conjugate or a fusion protein mixture thereof.

The targeting agent can be a protein or peptide fragment, such as a tissue specific or tumor specific monoclonal antibody or growth factor or fragment thereof linked either directly or via a linker to an MTSP20 polypeptide or a protease domain thereof. The targeting agent can also be a protein or peptide fragment that contains a protein binding sequence, a nucleic acid binding sequence, a lipid binding sequence, a polysaccharide binding sequence, or a metal binding sequence, or a linker for attachment to a solid support. In a particular embodiment, the conjugate contains a) the MTSP20 or portion thereof, as described herein; and b) a targeting agent linked to the MTSP20 polypeptide directly or via a linker.

Conjugates, such as fusion proteins and chemical conjugates, of the MTSP20 polypeptide with a protein or peptide fragment (or plurality thereof) that functions, for example, to facilitate affinity isolation or purification of the MTSP20 polypeptide domain, attachment of the MTSP20 polypeptide domain to a surface, or detection of the MTSP20 polypeptide domain are provided. The conjugates can be produced by chemical conjugation, such as via thiol linkages, and can be produced by recombinant means as fusion proteins. In the fusion protein, the peptide or fragment thereof is linked to either the N-terminus or C-terminus of the MTSP20 polypeptide domain. In chemical conjugates the peptide or fragment thereof can be linked anywhere that conjugation can be effected, and there can be a plurality of such peptides or fragments linked to a single MTSP20 polypeptide domain or to a plurality thereof.

The targeting agent is for in vitro or in vivo delivery to a cell or tissue, and includes agents such as cell or tissue-specific antibodies, growth factors and other factors (including compounds) that bind to moieties expressed on specific cells; and other cell or tissue specific agents that promote directed delivery of a linked protein. The targeting agent can be one that specifically delivers the MTSP20 polypeptide to selected cells by interaction with a cell surface protein and internalization of conjugate or MTSP20 polypeptide portion thereof.

These conjugates are used in a variety of methods and are particularly suited for use in methods of activation of prodrugs, such as prodrugs that upon cleavage by the particular MTSP20, which is localized at or near the targeted cell or tissue, are cytotoxic. The prodrugs are administered prior to, or simultaneously with, or subsequently to the conjugate. Upon delivery to the targeted cells, the protease activates the prodrug, which then exhibits a therapeutic effect, such as a cytotoxic effect.

1. Conjugation

Conjugates with linked MTSP20 polypeptide domains can be prepared either by chemical conjugation, recombinant DNA technology, or combinations of recombinant expression and chemical conjugation. The MTSP20 polypeptide domains and the targeting agent can be linked in any orientation and more than one targeting agent and/or MTSP20 polypeptide domain can be present in a conjugate.

a. Fusion Proteins

Fusion proteins are provided herein. A fusion protein contains: a) one or a plurality of domains of an MTSP20 polypeptide and b) a targeting agent. The fusion proteins are generally produced by recombinant expression of nucleic acids that encode the fusion protein.

b. Chemical Conjugation

To effect chemical conjugation herein, the MTSP20 polypeptide domain is linked via one or more selected linkers or directly to the targeting agent. Chemical conjugation must be used if the targeted agent is other than a peptide or protein, such as a nucleic acid or a non-peptide drug. Any means known to those of skill in the art for chemically conjugating selected moieties can be used.

2. Linkers

Linkers for two purposes are contemplated herein. The conjugates can include one or more linkers between the MTSP20 polypeptide portion and the targeting agent. Additionally, linkers are used for facilitating or enhancing immobilization of an MTSP20 polypeptide or portion thereof on a solid support, such as a microtiter plate, silicon or silicon-coated chip, glass or plastic support, such as for high throughput solid phase screening protocols.

Any linker known to those of skill in the art for preparation of conjugates can be used herein. These linkers are typically used in the preparation of chemical conjugates; peptide linkers can be incorporated into fusion proteins.

Linkers can be any moiety suitable to associate a domain of MTSP20 polypeptide and a targeting agent. Such linkers and linkages include, but are not limited to, amino acid and peptide linkages, typically containing between one and about 60 amino acids, more generally between about 10 and 30 amino acids, chemical linkers, such as heterobifunctional cleavable cross-linkers, including but are not limited to, N-succinimidyl (4-iodoacetyl)-aminobenzoate, sulfosuccinimydil (4-iodoacetyl)-aminobenzoate, 4-succinimidyl-oxycarbonyl-a-(2-pyridyldithio)toluene, sulfosuccinimidyl-6-[a-methyl-a-(pyridyldithiol)-toluamido] hexanoate, N-succinimidyl-3-(-2-pyridyldithio)-proprionate, succinimidyl 6[3(-(-2-pyridyidithio)-proprionamido]hexanoate, sulfosuccinimidyl 6[3(-(-2-pyridyldithio)-propionamido]hexanoate, 3-(2pyridyldithio)-propionyl hydrazide, Ellman's reagent, dichlorotriazinic acid, and S-(2-thiopyridyl)-L-cysteine. Other linkers include, but are not limited to peptides and other moieties that reduce steric hindrance between the domain of MTSP20 polypeptide and the targeting agent, intracellular enzyme substrates, linkers that increase the flexibility of the conjugate, linkers that increase the solubility of the conjugate, linkers that increase the serum stability of the conjugate, photocleavable linkers and acid cleavable linkers.

Other exemplary linkers and linkages that are suitable for chemically linked conjugates include, but are not limited to, disulfide bonds, thioether bonds, hindered disulfide bonds, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid dihydrazide, that would be cleaved in more acidic intracellular compartments; cross linkers that are cleaved upon exposure to UV or visible light; and linkers, such as various domains, such as C_{H}1, C_{H}2, and C_{H}3, from the constant region of human igG, (see, Batra et al. Molecular Immunol., 30:379-386 (1993)). In some embodiments, several linkers can be included in order to take advantage of desired properties of each linker.

Chemical linkers and peptide linkers can be inserted by covalently coupling the linker to the domain of MTSP20 polypeptide and the targeting agent. The heterobifunctional agents, described below, can be used to effect such covalent coupling. Peptide linkers can also be linked by expressing DNA encoding the linker and therapeutic agent (TA), linker and targeted agent, or linker, targeted agent and therapeutic agent (TA) as a fusion protein. Flexible linkers and linkers that increase solubility of the conjugates are contemplated for use, either alone or with other linkers are also contemplated herein.

a. Acid Cleavable, Photocleavable and Heat Sensitive Linkers

Acid cleavable linkers, photocleavable and heat sensitive linkers can also be used, particularly where it can be necessary to cleave the domain of MTSP20 polypeptide to permit it to be more readily accessible to reaction. Acid cleavable linkers include, but are not limited to, bismaleimideothoxy propane; and adipic acid dihydrazide linkers (see, e.g., Fattom et al. (1992) Infection & Immun. 60:584-589) and acid labile transferrin conjugates that contain a sufficient portion of transferrin to permit entry into the intracellular transferrin cycling pathway (see, e.g., Welhöner et al. (1991) J. Biol. Chem. 266:4309-4314).

Photocleavable linkers are linkers that are cleaved upon exposure to light (see, e.g., Goldmacher et al. (1992) Bioconj. Chem. 3:104-107, which linkers are herein incorporated by reference), thereby releasing the targeted agent upon exposure to light. Photocleavable linkers that are cleaved upon exposure to light are known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages), thereby releasing the targeted agent upon exposure to light. Such linkers would have particular use in treating dermatological or ophthalmic conditions that can be exposed to light using fiber optics. After administration of the conjugate, the eye or skin or other body part can be exposed to light, resulting in release of the targeted moiety from the conjugate. Such photocleavable linkers are useful in connection with diagnostic protocols in which it is desirable to remove the targeting agent to permit rapid clearance from the body of the animal.

b. Other Linkers for Chemical Conjugation

Other linkers, include trityl linkers, particularly, derivatized trityl groups to generate a genus of conjugates that provide for release of therapeutic agents at various degrees of acidity or alkalinity. The flexibility thus afforded by the ability to preselect the pH range at which the therapeutic agent is released allows selection of a linker based on the known physiological differences between tissues in need of delivery of a therapeutic agent (see, e.g., U.S. Pat. No. 5,612,474). For example, the acidity of tumor tissues appears to be lower than that of normal tissues.

c. Peptide Linkers

The linker moieties can be peptides. Peptide linkers can be employed in fusion proteins and also in chemically linked conjugates. The peptide typically has from about 2 to about 60 amino acid residues, for example from about 5 to about 40, or from about 10 to about 30 amino acid residues. The length selected depends upon factors, such as the use for which the linker is included.

Peptide linkers are advantageous when the targeting agent is proteinaceous. For example, the linker moiety can be a flexible spacer amino acid sequence, such as those known in single-chain antibody research. Examples of such known linker moieties include, but are not limited to, peptides, such as (GlyₘSer)ₙ and (SerₘGly)ₙ, in which n is 1 to 6, including 1 to 4 and 2 to 4, and m is 1 to 6, including 1 to 4, and 2 to 4, enzyme cleavable linkers and others.

Additional linking moieties are described, for example, in Huston et al., Proc. Natl. Acad. Sci. U.S.A. 85:5879-5883, 1988; Whitlow, M., et al., Protein Engineering 6:989-995, 1993; Newton et al., Biochemistry 35:545-553, 1996; A. J. Cumber et al., Bioconj. Chem. 3:397-401, 1992; Ladurner et al., J. Mol. Biol. 273:330-337, 1997; and U.S. Pat. No. 4,894,443. In some embodiments, several linkers can be included in order to take advantage of desired properties of each linker.

3. Targeting Agents

Any agent that facilitates detection, immobilization, or purification of the conjugate is contemplated for use herein. For chemical conjugates any moiety that has such properties is contemplated; for fusion proteins, the targeting agent is a protein, peptide or fragment thereof that is sufficient to effect the targeting activity. Contemplated targeting agents include those that deliver the MTSP20 polypeptide or portion thereof to selected cells and tissues. Such agents include tumor specific monoclonal antibodies and portions thereof, growth factors, such as FGF, EGF, PDGF, VEGF, cytokines, including chemokines, and other such agents.

4. Nucleic Acids, Plasmids and Cells

Isolated nucleic acid fragments encoding fusion proteins are provided. The nucleic acid fragment that encodes the fusion protein includes: a) nucleic acid encoding a protease domain of an MTSP20 polypeptide; and b) nucleic acid encoding a protein, peptide or effective fragment thereof that facilitates: i) affinity isolation or purification of the fusion protein; ii) attachment of the fusion protein to a surface; or iii) detection of the fusion protein. Generally, the nucleic acid is DNA.

Plasmids for replication and vectors for expression that contain the above nucleic acid fragments are also provided. Cells containing the plasmids and vectors are also provided. The cells can be any suitable host including, but are not limited to, bacterial cells, yeast cells, fungal cells, plant cells, insect cell and animal cells. The nucleic acids, plasmids, and cells containing the plasmids can be prepared according to methods known in the art including any described herein.

Also provided are methods for producing the above fusion proteins. An exemplary method includes the steps of growing, for example, culturing the cells so that they proliferate, cells containing a plasmid encoding the fusion protein under conditions whereby the fusion protein is expressed by the cell, and recovering the expressed fusion protein. Methods for expressing and recovering recombinant proteins are well known in the art (See generally, Current Protocols in Molecular Biology (1998) §16, John Wiley & Sons, Inc.) and such methods can be used for expressing and recovering the expressed fusion proteins.

The recovered fusion proteins can be isolated or purified by methods known in the art such as centrifugation, filtration, chromatography, electrophoresis, immunoprecipitation, and other such methods, or by a combination thereof (See generally, Current Protocols in Molecular Biology (1998) §10, John Wiley & Sons, Inc.). Generally the recovered fusion protein is isolated or purified through affinity binding between the protein or peptide fragment of the fusion protein and an affinity binding moiety. As discussed in the above sections regarding the construction of the fusion proteins, any affinity binding pairs can be constructed and used in the isolation or purification of the fusion proteins. For example, the affinity binding pairs can be protein binding sequences/protein, DNA binding sequences/DNA sequences, RNA binding sequences/RNA sequences, lipid binding sequences/lipid, polysaccharide binding sequences/polysaccharide, or metal binding sequences/metal.

5. Immobilization and Supports or Substrates Therefor

In certain embodiments, where the targeting agents are designed for linkage to surfaces, the MTSP20 polypeptide can be attached by linkage such as ionic or covalent, non-covalent or other chemical interaction, to a surface of a support or matrix material. Immobilization can be effected directly or via a linker. The MTSP20 polypeptide can be immobilized on any suitable support, including, but not limited to, silicon chips, and other supports described herein and known to those of skill in he art. A plurality of MTSP20 polypeptide or protease domains thereof can be attached to a support, such as an array (i.e., a pattern of two or more) of conjugates on the surface of a silicon chip or other chip for use in high throughput protocols and formats.

It is also noted that the domains of the MTSP20 polypeptide can be linked directly to the surface or via a linker without a targeting agent linked thereto. Hence chips containing arrays of the domains of the MTSP20 polypeptide are also provided.

The matrix material or solid supports contemplated herein are generally any of the insoluble materials known to those of skill in the art to immobilize ligands and other molecules, and are those that are used in many chemical syntheses and separations. Such supports are used, for example, in affinity chromatography, in the immobilization of biologically active materials, and during chemical syntheses of biomolecules, including proteins, amino acids and other organic molecules and polymers. The preparation of and use of supports is well known to those of skill in this art; there are many such materials and preparations thereof known. For example, naturally-occurring support materials, such as agarose and cellulose, can be isolated from their respective sources, and processed according to known protocols, and synthetic materials can be prepared in accord with known protocols.

The supports are typically insoluble materials that are solid, porous, deformable, or hard, and have any required structure and geometry, including, but not limited to: beads, pellets, disks, capillaries, hollow fibers, needles, solid fibers, random shapes, thin films and membranes. Thus, the item can be fabricated from the matrix material or combined with it, such as by coating all or part of the surface or impregnating particles.

Typically, when the matrix is particulate, the particles are at least about 10-2000 μm, but can be smaller or larger, depending upon the selected application. Selection of the matrices is governed, at least in part, by their physical and chemical properties, such as solubility, functional groups, mechanical stability, surface area swelling propensity, hydrophobic or hydrophilic properties and intended use.

If necessary, the support matrix material can be treated to contain an appropriate reactive moiety. In some cases, the support matrix material already containing the reactive moiety can be obtained commercially. The support matrix material containing the reactive moiety can thereby serve as the matrix support upon which molecules are linked. Materials containing reactive surface moieties such as amino silane linkages, hydroxyl linkages or carboxysilane linkages can be produced by well established surface chemistry techniques involving silanization reactions, or the like. Examples of these materials are those having surface silicon oxide moieties, covalently linked to gamma-aminopropylsilane, and other organic moieties; N-[3-(triethyoxysilyl)propyl]phthelamic acid; and bis-(2-hydroxyethyl)aminopropyltriethoxysilane. Exemplary readily available materials containing amino group reactive functionalities, include, but are not limited to, para-aminophenyltriethyoxysilane. Also derivatized polystyrenes and other such polymers are well known and readily available to those of skill in this art (e.g., the Tentagel® Resins are available with a multitude of functional groups, and are sold by Rapp Polymere, Tubingen, Germany; see, U.S. Pat. No. 4,908,405 and U.S. Pat. No. 5,292,814; see, also Butz et al., Peptide Res., 7:20-23 (1994); and Kleine et al., Immunobiol., 190:53-66 (1994)).

These matrix materials include any material that can act as a support matrix for attachment of the molecules of interest. Such materials are known to those of skill in this art, and include those that are used as a support matrix. These materials include, but are not limited to, inorganics, natural polymers, and synthetic polymers, including, but not limited to: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene and others (see, Merrifield, Biochemistry, 3:1385-1390 (1964)), polyacrylamides, latex gels, polystyrene, dextran, polyacrylamides, rubber, silicon, plastics, nitrocellulose, celluloses, natural sponges. Of particular interest herein, are highly porous glasses (see, e.g., U.S. Pat. No. 4,244,721) and others prepared by mixing a borosilicate, alcohol and water.

Synthetic supports include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield, Biochemistry, 3:1385-1390 (1964); Berg et al., in Innovation Perspect. Solid Phase Synth. Collect. Pap., Int. Symp., 1st, Epton, Roger (Ed), pp. 453-459 (1990); Berg et al., Pept., Proc. Eur. Pept. Symp., 20th, Jung, G. et al. (Eds), pp. 196-198 (1989); Berg et al., J. Am. Chem. Soc., 111:8024-8026 (1989); Kent et al., Isr. J. Chem., 17:243-247 (1979); Kent et al., J. Org. Chem., 43:2845-2852 (1978); Mitchell et al., Tetrahedron Lett., 42:3795-3798 (1976); U.S. Pat. No. 4,507,230; U.S. Pat. No. 4,006,117; and U.S. Pat. No. 5,389,449). Such materials include those made from polymers and co-polymers such as polyvinylalcohols, acrylates and acrylic acids such as polyethylene-co-acrylic acid, polyethylene-co-methacrylic acid, polyethylene-co-ethylacrylate, polyethylene-co-methyl acrylate, polypropylene-co-acrylic acid, polypropylene-co-methyl-acrylic acid, polypropylene-co-ethyl-acrylate, polypropylene-co-methyl acrylate, polyethylene-co-vinyl acetate, polypropylene-co-vinyl acetate, and those containing acid anhydride groups such as polyethylene-co-maleic anhydride and polypropylene-co-maleic anhydride. Liposomes have also been used as solid supports for affinity purifications (Powell et al. Biotechnol. Bioeng., 33:173 (1989)).

Numerous methods have been developed for the immobilization of proteins and other biomolecules onto solid or liquid supports (see, e.g., Mosbach, Methods in Enzymology, 44 (1976); Weetall, Immobilized Enzymes, Antigens, Antibodies, and Peptides, (1975); Kennedy et al., Solid Phase Biochemistry, Analytical and Synthetic Aspects, Scouten, ed., pp. 253-391 (1983); see, generally, Affinity Techniques. Enzyme Purification: Part B. Methods in Enzymology, Vol. 34, ed. W. B. Jakoby, M. Wilchek, Acad. Press, N.Y. (1974); and Immobilized Biochemicals and Affinity Chromatography, Advances in Experimental Medicine and Biology, vol. 42, ed. R. Dunlap, Plenum Press, N.Y. (1974)).

Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, ImmunoTechnology Catalog & Handbook, 1992-1993, which describes the preparation of and use of such reagents and provides a commercial source for such reagents; Wong, Chemistry of Protein Conjugation and Cross Linking, CRC Press (1993); see also DeWitt et al., Proc. Natl. Acad. Sci. U.S.A., 90:6909 (1993); Zuckermann et al., J. Am. Chem. Soc., 114:10646 (1992); Kurth et al., J. Am. Chem. Soc., 116:2661 (1994); Ellman et al., Proc. Natl. Acad. Sci. U.S.A., 91:4708 (1994); Sucholeiki, Tetrahedron Lttrs., 35:7307 (1994); Su-Sun Wang, J. Org. Chem., 41:3258 (1976); Padwa et al., J. Org. Chem., 41:3550 (1971); and Vedejs et al., J. Org. Chem., 49:575 (1984), which describe photosensitive linkers).

To effect immobilization, a composition containing the protein or other biomolecule is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO/86 03840).

J. Prognosis and Diagnosis

MTSP20 polypeptide proteins, domains, analogs, and derivatives thereof, and encoding nucleic acids (and sequences complementary thereto), and anti-MTSP20 polypeptide antibodies, can be used in diagnostics, particularly diagnosis of lung, head and neck, such as esophageal tumors, prostate, colon, ovary, cervix, breast and pancreas cancers. Such molecules can be used in assays, such as immunoassays, to detect, prognose, diagnose, or monitor various conditions, diseases, and disorders affecting MTSP20 polypeptide expression, or monitor the treatment thereof. For purposes herein, the presence of MTSP20s in body fluids or tumor tissues are of particular interest.

In particular, such an immunoassay is carried out by a method including contacting a sample derived from a patient with an anti-MTSP20 polypeptide antibody under conditions such that specific binding can occur, and detecting or measuring the amount of any specific binding by the antibody. Such binding of antibody, in tissue sections, can be used to detect aberrant MTSP20 polypeptide localization or aberrant (e.g., increased, decreased or absent) levels of MTSP20 polypeptide. In a specific embodiment, antibody to an MTSP20 polypeptide can be used to assay in a patient tissue or body fluid, such as serum, sample for the presence of MTSP20 polypeptide where an aberrant level of MTSP20 polypeptide is an indication of a diseased condition.

The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

MTSP20 polypeptide genes and related nucleic acid sequences and subsequences, including complementary sequences, also can be used in hybridization assays. MTSP20 polypeptide nucleic acid sequences, or subsequences thereof containing about at least 8 nucleotides, generally 14 or 16 or 30 or more, generally less than 1000 or up to 100, contiguous nucleotides can be used as hybridization probes. Hybridization assays can be used to detect, prognose, diagnose, or monitor conditions, disorders, or disease states associated with aberrant changes in MTSP20 polypeptide expression and/or activity as described herein. In particular, such a hybridization assay is carried out by a method of contacting a sample containing nucleic acid with a nucleic acid probe capable of hybridizing to MTSP20 polypeptide encoding DNA or RNA, under conditions such that hybridization can occur, and detecting or measuring any resulting hybridization.

In a specific embodiment, a method of diagnosing a disease or disorder characterized by detecting an aberrant level of an MTSP20 polypeptide in a subject is provided herein by measuring the level of the DNA, RNA, protein or functional activity of the MTSP20 polypeptide in a sample derived from the subject, wherein an increase or decrease in the level of the DNA, RNA, protein or functional activity of the MTSP20 polypeptide, relative to the level of the DNA, RNA, protein or functional activity found in an analogous sample not having the disease or disorder indicates the presence of the disease or disorder in the subject.

Kits for diagnostic use are also provided, that contain in one or more containers an anti-MTSP20 polypeptide antibody, and, optionally, a labeled binding partner to the antibody. Alternatively, the anti-MTSP20 polypeptide antibody can be labeled (with a detectable marker, e.g., a chemiluminescent, enzymatic, fluorescent, or radioactive moiety). A kit is also provided that includes in one or more containers a nucleic acid probe capable of hybridizing to the MTSP20 polypeptide-encoding nucleic acid. In a specific embodiment, a kit can include in one or more containers a pair of primers (e.g., each in the size range of 6-30 nucleotides) that are capable of priming amplification, e.g., by polymerase chain reaction (see e.g., Innis et al., 1990, PCR Protocols, Academic Press, Inc., San Diego, Calif.), ligase chain reaction (see EP 320,308), use of Qβ replicase, cyclic probe reaction, or other methods known in the art) under appropriate reaction conditions of at least a portion of an MTSP20 polypeptide-encoding nucleic acid. A kit can optionally further include in a container a predetermined amount of a purified MTSP20 polypeptide or nucleic acid, e.g., for use as a standard or control.

K. Pharmaceutical Compositions and Modes of Administration

1. Components of the Compositions

Pharmaceutical compositions containing the identified compounds that modulate an activity of an MTSP20 polypeptide are provided herein. Also provided are combinations of a compound that modulates an activity of an MTSP20 polypeptide and another treatment or compound for treatment of a neoplastic disorder, such as a chemotherapeutic compound.

The MTSP20 polypeptide modulator and the anti-tumor agent can be packaged as separate compositions for administration together or sequentially or intermittently. Alternatively, they can be provided as a single composition for administration or as two compositions for administration as a single composition. The combinations can be packaged as kits.

a. MTSP20 Polypeptide Inhibitors

Any MTSP20 polypeptide inhibitors, including those described herein when used alone or in combination with other compounds, that can alleviate, reduce, ameliorate, prevent, or place or maintain in a state of remission of clinical symptoms or diagnostic markers associated with neoplastic diseases, including undesired and/or uncontrolled angiogenesis, can be used in the present combinations.

In one embodiment, the MTSP20 polypeptide inhibitor is an antibody or fragment thereof that specifically reacts with an MTSP20 polypeptide or a protease domain thereof, an inhibitor of the MTSP20 polypeptide production, an inhibitor of MTSP20 polypeptide membrane-localization, or any inhibitor of the expression of or, especially, an activity of an MTSP20 polypeptide.

b. Anti-Angiogenic Agents and Anti-Tumor Agents

Any anti-angiogenic agents and anti-tumor agents, including those described herein, when used alone or in combination with other compounds, that can alleviate, reduce, ameliorate, prevent, or place or maintain in a state of remission of clinical symptoms or diagnostic markers associated with undesired and/or uncontrolled angiogenesis and/or tumor growth and metastasis, particularly solid neoplasms, vascular malformations and cardiovascular disorders, chronic inflammatory diseases and aberrant wound repairs, circulatory disorders, crest syndromes, dermatological disorders, or ocular disorders, can be used in the combinations. Also contemplated are anti-tumor agents for use in combination with an inhibitor of an MTSP20 polypeptide.

c. Pro-Angiogenic Agent

Any pro-angiogenic agents, including those described herein, when used alone or in combination with other compounds, that can promote physiological angiogenesis, particularly angiogenesis involved in normal placental, embryonic, fetal and post-natal development and growth, physiologically cyclical development in the ovarian follicle, corpus luteum and post-menstrual endometrium or wound healing, can be used in the present combinations.

The pro-angiogenic agent used in the combination can be a pro-angiogenic cytokine (Desai and Libutti, J. Immunother., 22(3):186-211 (1999)). For ecample, the pro-angiogenic cytokine used can be basic fibroblast growth factor such as bFGF and FGF-2, a vascular endothelial growth factor/vascular permeability factor such as VEGF/VPF and vasculotropin, a platelet-derived endothelial cell growth factor such as PD-EDGF and thymidine phosphorylase, a transforming growth factor-beta (TGF-β), or angiopoietin-1 (Ang-1).

d. Anti-Tumor Agents and Anti-Angiogenic Agents

The compounds identified by the methods provided herein or provided herein can be used in combination with anti-tumor agents and/or anti-angiogenesis agents and/or pro-angiogenic agents.

2. Formulations and Route of Administration

The compounds herein and agents can be formulated as pharmaceutical compositions, typically for single dosage administration. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds can be formulated as the sole pharmaceutically active ingredient in the composition or can be combined with other active ingredients. Liposomal suspensions, including tissue-targeted liposomes, can also be suitable as pharmaceutically acceptable carriers. These can be prepared according to methods known to those skilled in the art. For example, liposome formulations can be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration can be determined empirically by testing the compounds in known in vitro and in vivo systems, such as the assays provided herein.

The concentration of active compound in the drug composition depends on absorption, inactivation and excretion rates of the active compound, the physicochemical characteristics of the compound, the dosage schedule, and amount administered as well as other factors known to those of skill in the art.

Typically a therapeutically effective dosage is contemplated. The amounts administered can be on the order of 0.001 to 1 mg/ml, including about 0.005-0.05 mg/ml and about 0.01 mg/ml, of blood volume. Pharmaceutical dosage unit forms are prepared to provide from about 1 mg to about 1000 mg, including from about 10 to about 500 mg, and including about 25-75 mg of the essential active ingredient or a combination of essential ingredients per dosage unit form. The precise dosage can be empirically determined.

The active ingredient can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of the claimed compositions and combinations containing them.

Pharmaceutically acceptable derivatives include acids, salts, esters, hydrates, solvates and prodrug forms. The derivative is typically selected such that its pharmacokinetic properties are superior to the corresponding neutral compound.

Thus, effective concentrations or amounts of one or more of the compounds provided herein or pharmaceutically acceptable derivatives thereof are mixed with a suitable pharmaceutical carrier or vehicle for systemic, topical or local administration to form pharmaceutical compositions. Compounds are included in an amount effective for ameliorating or treating the disorder for which treatment is contemplated. The concentration of active compound in the composition depends on absorption, inactivation, excretion rates of the active compound, the dosage schedule, amount administered, particular formulation as well as other factors known to those of skill in the art.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds can be used. Such methods are known to those of skill in this art, and include, but are not limited to, using cosolvents, such as dimethylsulfoxide (DMSO), using surfactants, such as Tween®, or dissolution in aqueous sodium bicarbonate. Derivatives of the compounds, such as prodrugs of the compounds can also be used in formulating effective pharmaceutical compositions. For ophthalmic indications, the compositions are formulated in an ophthalmically acceptable carrier. For the ophthalmic uses herein, local administration, either by topical administration or by injection are contemplated. Time release formulations are also desirable. Typically, the compositions are formulated for single dosage administration, so that a single dose administers an effective amount.

Upon mixing or addition of the compound with the vehicle, the resulting mixture can be a solution, suspension, emulsion or other composition. The form of the resulting mixture depends upon a number of factors, including the intended mode of administration and the solubility of the compound in the selected carrier or vehicle. If necessary, pharmaceutically acceptable salts or other derivatives of the compounds are prepared.

The compound is included in the pharmaceutically acceptable carrier in an amount sufficient to exert a therapeutically useful effect in the absence of undesirable side effects on the patient treated. It is understood that number and degree of side effects depends upon the condition for which the compounds are administered. For example, certain toxic and undesirable side effects are tolerated when treating life-threatening illnesses that would not be tolerated when treating disorders of lesser consequence.

The compounds also can be mixed with other active materials, that do not impair the desired action, or with materials that supplement the desired action known to those of skill in the art. The formulations of the compounds and agents for use herein include those suitable for oral, rectal, topical, inhalational, buccal (e.g., sublingual), parenteral (e.g., subcutaneous, intramuscular, intradermal, or intravenous), transdermal administration or any route. The most suitable route in any given case depends on the nature and severity of the condition being treated and on the nature of the particular active compound which is being used. The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethyl-cellulose; a lubricant, such as magnesium stearate, calcium stearate and talc; and a binder such as starch, natural gums, such as gum acacia, gelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Methods of preparing such dosage forms are known, or will be apparent, to those skilled in this art (see, e.g., Remington's Pharmaceutical Sciences, Mack Publishing Company, Easton, Pa., 15th Edition, 1975). The composition or formulation to be administered contains a quantity of the active compound in an amount sufficient to alleviate the symptoms of the treated subject.

Dosage forms or compositions containing active ingredient in the range of 0.005% to 100% with the balance made up from non-toxic carrier can be prepared. For oral administration, the pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well-known in the art.

The pharmaceutical preparation can also be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be presented as unit dose suppositories. These can be prepared by admixing the active compound with one or more conventional solid carriers, for example, cocoa butter, and then shaping the resulting mixture.

Formulations suitable for topical application to the skin or to the eye generally are formulated as an ointment, cream, lotion, paste, gel, spray, aerosol and oil. Carriers which can be used include vaseline, lanoline, polyethylene glycols, alcohols, and combinations of two or more thereof. The topical formulations can further advantageously contain 0.05 to 15 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It can also be injected into the anterior eye chamber and other places. The topical formulations in the liquid state can be also present in a hydrophilic three-dimensional polymer matrix in the form of a strip, contact lens, and the like from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

The compounds can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the active compound. Formulations suitable for transdermal administration can also be delivered by iontophoresis (see, e.g., Pharmaceutical Research 3 (6), 318 (1986)) and typically take the form of an optionally buffered aqueous solution of the active compound.

The pharmaceutical compositions can also be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects).

The efficacy and/or toxicity of the MTSP20 polypeptide inhibitor(s), alone or in combination with other agents also can be assessed by the methods known in the art (see generally, O'Reilly, Investigational New Drugs, 15:5-13 (1997)).

The active compounds or pharmaceutically acceptable derivatives can be prepared with carriers that protect the compound against rapid elimination from the body, such as time release formulations or coatings.

Kits containing the compositions and/or the combinations with instructions for administration thereof are provided. The kit can further include a needle or syringe, typically packaged in sterile form, for injecting the complex, and/or a packaged alcohol pad. Instructions are optionally included for administration of the active agent by a clinician or by the patient.

Finally, the compounds or MTSP20 polypeptides or protease domains thereof or compositions containing any of the preceding agents can be packaged as articles of manufacture containing packaging material, a compound or suitable derivative thereof provided herein, which is effective for treatment of diseases or disorders contemplated herein, within the packaging material, and a label that indicates that the compound or a suitable derivative thereof is for treating the diseases or disorders contemplated herein. The label can optionally include the disorders for which the therapy is warranted.

L. Methods of Treatment

The compounds identified by the methods herein are used for treating or preventing neoplastic diseases in an animal, particularly a mammal, including a human, as provided herein. In one embodiment, the method includes administering to a mammal an effective amount of an inhibitor of an MTSP20 polypeptide, whereby the disease or disorder is treated or prevented.

In an embodiment, the MTSP20 polypeptide inhibitor used in the treatment or prevention is administered with a pharmaceutically acceptable carrier or excipient. The mammal treated can be a human. The inhibitors provided herein are those identified by the screening assays. In addition, antibodies and antisense nucleic acids or double-stranded RNA (dsRNA), such as RNAi, are contemplated.

The treatment or prevention method can further include administering an anti-angiogenic treatment or agent or anti-tumor agent simultaneously with, prior to or subsequent to the MTSP20 polypeptide inhibitor, which can be any compound identified that inhibits an activity of an MTSP20 polypeptide. Such compounds include small molecule modulators, an antibody or a fragment or derivative thereof containing a binding region thereof against the MTSP20 polypeptide, an antisense nucleic acid or double-stranded RNA (dsRNA), such as RNAi, encoding an a portion of the MTSP20 polypeptide or complementary to thereto, and a nucleic acid containing at least a portion of a gene encoding the MTSP20 polypeptide into which a heterologous nucleotide sequence has been inserted such that the heterologous sequence inactivates the biological activity of at least a portion of the gene encoding the MTSP20 polypeptide, in which the portion of the gene encoding the MTSP20 polypeptide flanks the heterologous sequence to promote homologous recombination with a genomic gene encoding the MTSP20 polypeptide. In addition, such molecules are generally less than about 1000 nt long.

1. Antisense Treatment

In a specific embodiment, as described hereinabove, MTSP20 polypeptide function is reduced or inhibited by MTSP20 polypeptide antisense nucleic acids, to treat or prevent neoplastic disease. The therapeutic or prophylactic use of nucleic acids of at least six nucleotides, generally up to about 150 nucleotides, that are antisense to a gene or cDNA encoding MTSP20 polypeptide or a portion thereof is provided. An MTSP20 polypeptide “antisense” nucleic acid as used herein refers to a nucleic acid capable of hybridizing to a portion of an MTSP20 polypeptide RNA (generally mRNA) by virtue of some sequence complementarity, and generally under high stringency conditions. The antisense nucleic acid can be complementary to a coding and/or noncoding region of an MTSP20 polypeptide mRNA. Such antisense nucleic acids have utility as therapeutics that reduce or inhibit MTSP20 polypeptide function, and can be used in the treatment or prevention of disorders as described supra.

The MTSP20 polypeptide antisense nucleic acids are of at least six nucleotides and are generally oligonucleotides (ranging from 6 to about 150 nucleotides including 6 to 50 nucleotides). The antisense molecule can be complementary to all or a portion of a protease domain. For example, the oligonucleotide is at least 10 nucleotides, at least 15 nucleotides, at least 100 nucleotides, or at least 125 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide can include other appending groups such as peptides, or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. U.S.A. 84:648-652 (1987); PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)).

The MTSP20 polypeptide antisense nucleic acid generally is an oligonucleotide, typically single-stranded DNA or RNA or an analog thereof or mixtures thereof. For example, the oligonucleotide includes a sequence antisense to a portion of a nucleic acid that encodes a human MTSP20 polypeptide. The oligonucleotide can be modified at any position on its structure with substituents generally known in the art.

The MTSP20 polypeptide antisense oligonucleotide can include at least one modified base moiety which is selected from the group including, but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine.

In another embodiment, the oligonucleotide includes at least one modified sugar moiety selected from the group including but not limited to arabinose, 2-fluoroarabinose, xylulose, and hexose. The oligonucleotide can include at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analog thereof.

The oligonucleotide can be an a-anomeric oligonucleotide. An a-anomeric oligonucleotide forms specific double-stranded hybrids with complementary RNA in which the strands run parallel to each other (Gautier et al., Nucl. Acids Res. 15:6625-6641 (1987)).

The oligonucleotide can be conjugated to another molecule, such as, but are not limited to, a peptide, hybridization triggered cross-linking agent, transport agent or a hybridization-triggered cleavage agent. The oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (Nucl. Acids Res. 16:3209 (1988)), methylphosphonate oligonucleotides can be prepared by use of controlled pore glass polymer supports (Sarin et al., Proc. Natl. Acad. Sc. U.S.A. 85:7448-7451 (1988)), etc.

In a specific embodiment, the MTSP20 polypeptide antisense oligonucleotide includes catalytic RNA or a ribozyme (see, e.g., PCT International Publication WO 90/11364, published Oct. 4, 1990; Sarver et al., Science 247:1222-1225 (1990)). In another embodiment, the oligonucleotide is a 2′-O-methylribonucleotide (Inoue et al., Nucl. Acids Res. 15:6131-6148 (1987)), or a chimeric RNA-DNA analogue (Inoue et al., FEBS Lett. 215:327-330 (1987)). Alternatively, the oligonucleotide can be double-stranded RNA (dsRNA) such as RNAi.

In an alternative embodiment, the MTSP20 polypeptide antisense nucleic acid is produced intracellularly by transcription from an exogenous sequence. For example, a vector can be introduced in vivo such that it is taken up by a cell, within which cell the vector or a portion thereof is transcribed, producing an antisense nucleic acid (RNA). Such a vector would contain a sequence encoding the MTSP20 polypeptide antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the MTSP20 polypeptide antisense RNA can be by any promoter known in the art to act in mammalian, including human, cells. Such promoters can be inducible or constitutive. Such promoters include but are not limited to: the SV40 early promoter region (Bernoist and Chambon, Nature 290:304-310 (1981), the promoter contained in the 3′ long terminal repeat of Rous sarcoma virus (Yamamoto et al., Cell 22:787-797 (1980), the herpes thymidine kinase promoter (Wagner et al., Proc. Natl. Acad. Sci. U.S.A. 78:1441-1445 (1981), the regulatory sequences of the metallothionein gene (Brinster et al., Nature 296:39-42 (1982), etc.

The antisense nucleic acids include sequence complementary to at least a portion of an RNA transcript of an MTSP20 polypeptide gene, including a human MTSP20 polypeptide gene. Absolute complementarily is not required.

The amount of MTSP20 polypeptide antisense nucleic acid that is effective in the treatment or prevention of neoplastic disease depends on the nature of the disease, and can be determined empirically by standard clinical techniques. Where possible, it is desirable to determine the antisense cytotoxicity in cells in vitro, and then in useful animal model systems prior to testing and use in humans.

2. RNA Interference

RNA interference (RNAi) (see, e.g. Chuang et al. (2000) Proc. Natl. Acad. Sci. U.S.A. 97:4985) can be employed to inhibit the expression of a gene encoding an MTSP20. Interfering RNA (RNAi) fragments, particularly double-stranded (ds) RNAi, can be used to generate loss-of-MTSP20 function. Methods relating to the use of RNAi to silence genes in organisms including, mammals, C. elegans, Drosophila and plants, and humans are known (see, e.g., Fire et al. (1998) Nature 391:806-811 Fire (1999) Trends Genet. 15:358-363; Sharp (2001) Genes Dev. 15:485-490; Hammond, et al. (2001) Nature Rev. Genet.2:110-1119; Tuschl (2001) Chem. Biochem. 2:239-245; Hamilton et al. (1999) Science 286:950-952; Hammond et al. (2000) Nature 404:293-296; Zamore et al. (2000) Cell 101:25-33; Bernstein et al. (2001) Nature 409: 363-366; Elbashir et al. (2001) Genes Dev. 15:188-200; Elbashir et al. (2001) Nature 411:494-498; International PCT application No. WO 01/29058; International PCT application No. WO 99/32619).

Double-stranded RNA (dsRNA)-expressing constructs are introduced into a host, such as an animal or plant using, a replicable vector that remains episomal or integrates into the genome. By selecting appropriate sequences, expression of dsRNA can interfere with accumulation of endogenous mRNA encoding an MTSP20. RNAi also can be used to inhibit expression in vitro. Regions including at least about 21 (or 21) nucleotides that are selective (i.e. unique) for MTSP20 are used to prepare the RNAi. Smaller fragments of about 21 nucleotides can be transformed directly (i.e., in vitro or in vivo) into cells; larger RNAi dsRNA molecules are generally introduced using vectors that encode them. dsRNA molecules are at least about 21 bp long or longer, such as 50, 100, 150, 200 and longer. Methods, reagents and protocols for introducing nucleic acid molecules into cells in vitro and in vivo are known to those of skill in the art.

3. Gene Therapy

In an exemplary embodiment, nucleic acids that include a sequence of nucleotides encoding an MTSP20 polypeptide or functional domains or derivative thereof, are administered to promote MTSP20 polypeptide function, by way of gene therapy. Gene therapy refers to therapy performed by the administration of a nucleic acid to a subject. In this embodiment, the nucleic acid produces its encoded protein that mediates a therapeutic effect by promoting MTSP20 polypeptide function. Any of the methods for gene therapy available in the art can be used (see, Goldspiel et al., Clinical Pharmacy 12:488-505 (1993); Wu and Wu, Biotherapy 3:87-95 (1991); Tolstoshev, An. Rev. Pharmacol. Toxicol. 32:573-596 (1993); Mulligan, Science 260:926-932 (1993); and Morgan and Anderson, An. Rev. Biochem. 62:191-217 (1993); TIBTECH 11(5):155-215 (1993).

For example, one therapeutic composition for gene therapy includes an MTSP20 polypeptide-encoding nucleic acid that is part of an expression vector that expresses an MTSP20 polypeptide or domain, fragment or chimeric protein thereof in a suitable host. In particular, such a nucleic acid has a promoter operably linked to the MTSP20 polypeptide coding region, the promoter being inducible or constitutive, and, optionally, tissue-specific. In another particular embodiment, a nucleic acid molecule is used in which the MTSP20 polypeptide coding sequences and any other desired sequences are flanked by regions that promote homologous recombination at a desired site in the genome, thus providing for intrachromosomal expression of the SP protein nucleic acid (Koller and Smithies, Proc. Natl. Acad. Sci. USA 86:8932-8935 (1989); Zijistra et al., Nature 342:435-438 (1989)).

Delivery of the nucleic acid into a patient can be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, J. Biol. Chem. 262:4429-4432 (1987)) (which can be used to target cell types specifically expressing the receptors), etc. In another embodiment, a nucleic acid-ligand complex can be formed in which the ligand is a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, a viral vector that contains the MTSP20 polypeptide nucleic acid is used. For example, a retroviral vector can be used (see Miller et al., Meth. Enzymol. 217:581-599 (1993)). These retroviral vectors have been modified to delete retroviral sequences that are not necessary for packaging of the viral genome and integration into host cell DNA. The MTSP20 polypeptide nucleic acid to be used in gene therapy is cloned into the vector, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); and Mastrangeli et al., J. Clin. Invest. 91:225-234 (1993).

Adeno-associated virus (AAV) has also been proposed for use in gene therapy (Walsh et al. (1993) Proc. Soc. Exp. Biol. Med. 204:289-300).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, Meth. Enzymol. 217:599-618 (1993); Cohen et al., Meth. Enzymol. 217:618-644 (1993); Cline, Pharmac. Ther. 29:69-92 (1985)) and can be used, provided that the necessary developmental and physiological functions of the recipient cells are not disrupted. The technique should provide for the stable transfer of the nucleic acid to the cell, so that the nucleic acid is expressible by the cell and generally heritable and expressible by its cell progeny.

The resulting recombinant cells can be delivered to a patient by various methods known in the art. In an embodiment, epithelial cells are injected, e.g., subcutaneously. In another embodiment, recombinant skin cells can be applied as a skin graft onto the patient. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For example, a cell used for gene therapy is autologous to the patient. In an embodiment in which recombinant cells are used in gene therapy, an MTSP20 polypeptide nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment. Such stem cells include but are not limited to hematopoietic stem cells (HSC), stem cells of epithelial tissues such as the skin and the lining of the gut, embryonic heart muscle cells, liver stem cells (PCT Publication WO 94/08598, dated Apr. 28, 1994), and neural stem cells (Stemple and Anderson, Cell 71:973-985 (1992)).

Epithelial stem cells (ESCs) or keratinocytes can be obtained from tissues such as the skin and the lining of the gut by known procedures (Rheinwald, Meth. Cell Bio. 21A:229 (1980)). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, Meth. Cell Bio. 21A:229 (1980); Pittelkow and Scott, Cano Clinic Proc. 61:771 (1986)). If the ESCs are provided by a donor, a method for suppression of host versus graft reactivity (e.g., irradiation, drug or antibody administration to promote moderate immunosuppression) also can be used.

With respect to hematopoietic stem cells (HSC), any technique which provides for the isolation, propagation, and maintenance in vitro of HSC can be used in this embodiment. Techniques by which this can be accomplished include (a) the isolation and establishment of HSC cultures from bone marrow cells isolated from the future host, or a donor, or (b) the use of previously established long-term HSC cultures, which can be allogeneic or xenogeneic. Non-autologous HSC generally are used with a method of suppressing transplantation immune reactions of the future host/patient. In a particular embodiment, human bone marrow cells can be obtained from the posterior iliac crest by needle aspiration (see, e.g., Kodo et al., J. Clin. Invest. 73:1377-1384 (1984)). For example, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., J. Cell Physiol. 91:335 (1977) or Witlock-Witte culture techniques (Witlock and Witte, Proc. Natl. Acad. Sci. USA 79:3608-3612 (1982)).

In a specific embodiment, the nucleic acid to be introduced for purposes of gene therapy includes an inducible promoter operably linked to the coding region, such that expression of the nucleic acid is controllable by controlling the presence or absence of the appropriate inducer of transcription.

4. Prodrugs

A method for treating tumors is provided. The method is practiced by administering a prodrug that is cleaved at a specific site by an MTSP20 to release an active drug or precursor that can be converted to active drug in vivo. Upon contact with a cell that expresses MTSP20 activity, the prodrug is converted into an active drug. The prodrug can be a conjugate that contains the active agent, such as an anti-tumor drug, such as a cytotoxic agent, or other therapeutic agent (TA), linked to a substrate for the targeted MTSP20, such that the drug or agent is inactive or unable to enter a cell, in the conjugate, but is activated upon cleavage. The prodrug, for example, can contain an oligopeptide, typically a relatively short, less than about 10 amino acids peptide, that is proteolytically cleaved by the targeted MTSP20. Cytotoxic agents, include, but are not limited to, alkylating agents, antiproliferative agents and tubulin binding agents. Others cytotoxic acids include, but are not limited to, vinca drugs, mitomycins, bleomycins and taxanes.

5. Treatment of Undesired Angiogenesis

Compounds and combinations provided herein can be used for treating or preventing a disease or disorder, including cancers and ophthalmic disorders and restenosis, associated with undesired and/or uncontrolled angiogenesis in a mammal. Methods using such compounds and combinations are provided. In one embodiment, the method includes administering to a mammal an effective amount of an inhibitor of the MTSP20, which is an endotheliase, whereby the disease or disorder is treated or prevented. In another embodiment, the endotheliase inhibitor used in the treatment or prevention is administered with a pharmaceutically acceptable carrier or excipient. The mammal treated can be a human.

In other embodiments, the treatment or prevention method further includes administering an anti-angiogenic treatment or agent simultaneously with, prior to or subsequent to the MTSP20 inhibitor, which can be any compound identified that inhibits an activity of MTSP20 expressed on endothelial cells, and includes an antibody or a fragment or derivative thereof containing the binding region thereof against the MTSP20 expressed on endothelial cells, an antisense nucleic acid encoding the MTSP20, and a nucleic acid containing at least a portion of a gene encoding the MTSP20, which is expressed on endothelial cells, into which a heterologous nucleotide sequence has been inserted such that the heterologous sequence inactivates the biological activity of at least a portion of the gene encoding the MTSP20, which is expressed on endothelial cells, in which the portion of the gene encoding the MTSP20 flanks the heterologous sequence so as to promote homologous recombination with a genomic gene encoding the MTSP20.

The undesired or aberrant angiogenesis to be treated or prevented is associated with solid neoplasms, vascular malformations and cardiovascular disorders, chronic inflammatory diseases and aberrant wound repairs, circulatory disorders, crest syndromes, dermatological disorders, or ocular disorders. Vascular malformations and cardiovascular disorders to be treated or prevented include angiofibroma, angiolipoma, atherosclerosis, restenosis/reperfusion injury, arteriovenous malformations, hemangiomatosis and vascular adhesions, dyschondroplasia with vascular hamartomas (Fafucci's syndrome), hereditary hemorrhagic telangiectasia (Rendu-Osler-Weber syndrome), or Von Hipple Lindau syndrome; the chronic inflammatory diseases to be treated or prevented are diabetes mellitus, hemophiliac joints, inflammatory bowel disease, nonhealing fractures, periodontitis (rapidly progressing and juvenile), psoriasis, rheumatoid arthritis, venous stasis ulcers, granulations-burns, hypertrophic scars, liver cirrhosis, osteoradionecrosis, postoperative adhesions, pyogenic granuloma, or systemic sclerosis; the circulatory disorder to be treated or prevented is Raynaud's phenomenon; the crest syndromes to be treated or prevented are calcinosis, esophageal, dyomotiloty, sclerodactyly and teangiectasis; the dermatological disorders to be treated or prevented are systemic vasculitis, scleroderma, pyoderma gangrenosum, vasculopathy, venous, arterial ulcers, Sturge-Weber syndrome, Port-wine stains, blue rubber bleb nevus syndrome, Klippel-Trenaunay-Weber syndrome or Osler-Weber-Rendu syndrome; and the ocular disorders to be treated or prevented are blindness caused by ocular neovascular disease, corneal graft neovascularization, macular degeneration in the eye, neovascular glaucoma, trachoma, diabetic retinopathy, myopic degeration, retinopathy of prematurity, retrolental fibroplasia, or corneal neovascularization.

6. Treatment of Deficient Angiogenesis

In another specific embodiment, a method for treating or preventing a disease or disorder associated with deficient angiogenesis in a mammal is provided herein by administering to a mammal an effective amount of an MTSP20 or a derivative or analog of the protein that is active in promoting angiogenesis, or a nucleic acid encoding the protein or a derivative or analog of the protein that is active in promoting angiogenesis, whereby the disease or disorder is treated or prevented. The MTSP20 or portion thereof or a derivative or analog of the protein can be administered to a subject, such as a mammal, including a human, with a pharmaceutically acceptable carrier or excipient.

The treatment or prevention methods can further include administering a pro-angiogenic treatment or agent. The treatment can be used to promote physiological angiogenesis, particularly angiogenesis involved in normal placental, embryonic, fetal and post-natal development and growth, physiologically cyclical development in the ovarian follicle, corpus luteum and post-menstrual endometrium or wound healing.

The pro-angiogenic agent used in the treatment can be, for example, a basic fibroblast growth factor such as bFGF and FGF-2, a vascular endothelial growth factor/vascular permeability factor such as VEGF/VPF and vasculotropin, a platelet-derived endothelial cell growth factor such as PD-EDGF and thymidine phosphorylase, a transforming growth factor-beta (TGF-β), or angiopoietin-1 (Ang-1).

M. Animal Models

Transgenic animal models and animals, such as rodents, including mice and rats, cows, chickens, pigs, goats, sheep, monkeys, including gorillas, and other primates, are provided herein. In particular, transgenic non-human animals that contain heterologous nucleic acid encoding an MTSP20 polypeptide or a transgenic animal in which expression of the polypeptide has been altered, such as by replacing or modifying the promoter region or other regulatory region of the endogenous gene are provided. Such an animal can by produced by promoting recombination between endogenous nucleic acid and an exogenous MTSP20 gene that could be over-expressed or mis-expressed, such as by expression under a strong promoter, via homologous or other recombination event.

Transgenic animals can be produced by introducing the nucleic acid using any known method of delivery, including, but not limited to, microinjection, lipofection and other modes of gene delivery into a germline cell or somatic cells, such as an embryonic stem cell. Typically the nucleic acid is introduced into a cell, such as an embryonic stem cell (ES), followed by injecting the ES cells into a blastocyst, and implanting the blastocyst into a foster mother, which is followed by the birth of a transgenic animal. Generally, introduction of a heterologous nucleic acid molecule into a chromosome of the animal occurs by a recombination between the heterologous MTSP20-encoding nucleic acid and endogenous nucleic acid. The heterologous nucleic acid can be targeted to a specific chromosome.

In some instances, knockout animals can be produced. Such an animal can be initially produced by promoting homologous recombination between an MTSP20 polypeptide gene in its chromosome and an exogenous MTSP20 polypeptide gene that has been rendered biologically inactive (typically by insertion of a heterologous sequence, e.g., an antibiotic resistance gene). In one embodiment, this homologous recombination is performed by transforming embryo-derived stem (ES) cells with a vector containing the insertionally inactivated MTSP20 polypeptide gene, such that homologous recombination occurs, followed by injecting the ES cells into a blastocyst, and implanting the blastocyst into a foster mother, followed by the birth of the chimeric animal (“knockout animal”) in which an MTSP20 polypeptide gene has been inactivated (see Capecchi, Science 244:1288-1292 (1989)). The chimeric animal can be bred to produce homozygous knockout animals, which can then be used to produce additional knockout animals. Knockout animals include, but are not limited to, mice, hamsters, sheep, pigs, cattle, and other non-human mammals. For example, a knockout mouse is produced. The resulting animals can serve as models of specific diseases, such as cancers, that exhibit under-expression of an MTSP20 polypeptide. Such knockout animals can be used as animal models of such diseases e.g., to screen for or test molecules for the ability to treat or prevent such diseases or disorders.

Other types of transgenic animals also can be produced, including those that over-express the MTSP20 polypeptide. Such animals include “knock-in” animals that are animals in which the normal gene is replaced by a variant, such as a mutant, an over-expressed form, or other form. For example, one species, such as a rodent's endogenous gene can be replaced by the gene from another species, such as from a human. Animals also can be produced by non-homologous recombination into other sites in a chromosome; including animals that have a plurality of integration events.

After production of the first generation transgenic animal, a chimeric animal can be bred to produce additional animals with over-expressed or mis-expressed MTSP20 polypeptides. Such animals include, but are not limited to, mice, hamsters, sheep, pigs, cattle and other non-human mammals. The resulting animals can serve as models of specific diseases, such as cancers, that are exhibit over-expression or mis-expression of an MTSP20 polypeptide. Such animals can be used as animal models of such diseases e.g., to screen for or test molecules for the ability to treat or prevent such diseases or disorders. In a specific embodiment, a mouse with over-expressed or mis-expressed MTSP20 polypeptide is produced.

The following examples are included for illustrative purposes only and are not intended to limit the scope of the invention.

### EXAMPLE 1

Identification of MTSP20

The protein sequence of the protease domain of endotheliase1 (ET1; also called DESC1, accession number XP_{—}003340) was used to search the human HTGS (High Throughput Genomic Sequence) database using the tblastn algorithm (http://www.ncbi.nlm.nih.gov/BLAST). This search and alignment algorithm compares a protein query sequence against a nucleotide sequence database dynamically translated in all six reading frames (both strands); MTSP20 was identified. MTSP20 shares 39% identity to a region of ET1 between aa 188 and aa 233, and 30% identity to a region of ET1 between aa 239 to aa 314. A search using the algorithm blastp (http://www.ncbi.nim.nih.gov/BLAST ) indicates that the translated sequence of these 2 regions in MTSP20 has ˜33% identity to the region flanking the catalytic aspartate of prostamin (accession number AB030036) and MTSP6 (see, International PCT application No. (WO 01/57194), and 45% identity with the region flanking the catalytic histidine of prostasin (accession number NP_{—}002764). Based on the incomplete and unordered human genome sequence, MTSP20 appears to be localized on chromosome 16 (clone accession number AC009088). A search of sequences deposited in GenBank showed that no identical sequence had been deposited. Further search of the human EST database showed 2 EST clones (IMAGE cDNA clone 3535196; GenBank accession number: BE264142 and IMAGE cDNA clone 4932600; GenBank accession number: BG819317) were identical to a portion of the MTSP20 sequence. IMAGE clone 3535196 is a publicly available clone derived from human small cell lung carcinoma, and this clone was purchased from Incyte Genomics (Palo Alto, Calif.) and sequenced. The sequence of this EST clone did not match the deposited sequence. Instead, it matched that of human Sck, a neuronal Shc adapter (GenBank accession number AB001451). The second publicly available EST clone (IMAGE 4932600) was derived from human oligodendroma, but was unavailable.

The MTSP20 and encoding nucleic acid has homology to a clone described in International PCT application No. WO 02/00860 (see SEQ ID Nos. 27 and 86 therein; and SEQ ID No. 17 herein). The clone and predicted encoded polypeptide described in the PCT application, however, differ from the nucleic acid molecule encoding MTSP20 pand the MTSP20 provided herein. The nucleic acid molecule includes nucleic acids encoding amino acids 624-642 absent from the clone in International PCT application No. WO 02/00860. Full length polypeptides, polypeptides containing two protease domains and polypeptides containing MTSPP20 PD2 are provided herein.

Cloning of MTSP20 from Human Lung Tissue

Using the genomic sequence of MTSP20, two gene specific oligonucleotide primers were designed. The sequence for the 5′ end primer is 5′-GAAGCAGCTCATCCTGCATGGAGCCTACAC-3′ SEQ ID No. 7 and that of the 3′ end primer is 5′-GGCCAGGAACCATGTGCCCCTCACCTC-3′ SEQ ID No. 8. These primers were used to amplify a cDNA fragment from human fetal lung tissue (part of Human Fetal Multiple Tissue cDNA Panel ; Clontech, Palo Alto, Calif.; catalog no. K1425-1). Several bands were detected. A band (˜400 bp) whose size closely matched that of the predicted, translated genomic sequence was isolated by 2% agarose gel electrophoresis, purified using the MinElute gel extraction kit (catalog number 28606; http://www.qiagen.com), subcloned into an E. coli vector (pCR2.1TOPO; catalog no. K-4500-01; http://www.invitrogen.com) and transformed into E. coli TOP10 cells (http://www.invitrogen.com). Subsequent sequence analysis using a fluorescent dye-based DNA sequencing method (catalog number 4390244; ABI PRISM® BigDye™ Terminator v 3.0 Ready Reaction Cycle Sequencing Kits with AmpliTaq® DNA Polymerase, FS; http://home.appliedbiosystems.com) showed that the nucleotide sequence of this cDNA fragment matched that of the genomic MTSP20 exon sequences.

5′-RACE and Gene-Specific PCR Amplification of cDNA Encoding Full-Length Protease Domain of MTSP20

Using the derived cDNA sequence of MTSP20, 2 oligonucleotide primers hybridizing within the protease domain sequence were designed and synthesized. The sequence for the 5′ end primer is 5′-GCTGCTTCAGGCCCCACTCCTCCGGTCTGG-3′ (SEQ ID No. 18), and that of the nested 5′ end primer is 5′-CACAGGCCAGCTGTCCCTGGTGCATCAGCC-3′ (SEQ ID No. 19). The initial 5′-RACE primer was used to amplify cDNA fragments from human lymph node Marathon-ready cDNA library (catalog number 7432-1; http://www.clontech.com). Following this reaction, a nested 5′-RACE reaction was performed using the nested primer. Several cDNA bands were detected in all RACE reactions. The cDNA fragments were separated by 2% agarose gel electrophoresis, blotted onto a nylon membrane (SuperCharge Nytran; http://www.s-und-s.de/Pages-NEU-eng/UB3/Life %20Science/nytran.htm) and hybridized with the ³²P-labeled cDNA fragment described previously.

Several cDNA bands hybridized with the probe. The 2 largest positive cDNA bands were purified from the nested 5′-RACE reaction using the MinElute gel extraction kit, subcloned into an E. coli vector (pCR2.1 TOPO) and transformed into E. coli TOP10 cells. Several independent clones were cultured and used to isolate plasmid DNA. Subsequent sequence analysis confirmed that the nucleotide sequence contained some of the missing 5′-end sequence of MTSP20. The 5′-RACE product did not extend to the beginning of the cDNA as the methionine start codon was missing.

To obtain the cDNA fragment encoding the rest of the 5′ end of MTSP20, a gene-specific PCR amplification using a cDNA library prepared from human aortic endothelial cells (HAEC-aortic endothelial cells; catalog number CC-2635; Clonetics, http://www.cambrex.com) was used. Two 5′-end primers were used: 5′-AGCACGGCCGGCCAATCGCCGAGTCAGAGG-3′ (SEQ ID No. 20) and 5′-GCCTCGGGCGGAACCTGGAGATAATGGGC-3′ (SEQ ID No. 21) (underlined sequence represents the putative start codon). The 1^{st 5}′-end primer was about 95-bp upstream of the 2^{nd 5}′-end primer and located in the 5′-UTR of MTSP20. For both PCR reactions, a single MTSP20-specific 3′-end primer was used: 5′-GACCAGGAATTCAGTTCTGTTGCTGCTGCC-3′ (SEQ ID No. 22). The 3′-end primer corresponds to the sequence found in the 5′-RACE product isolated and sequenced previously, and it occurs immediately downstream of the catalytic histidine.

Two cDNA fragments (˜600-bp and 510-bp) were isolated from the PCR reaction using the 1^{st} 5-end and 3′-end primer pair, and 2^{nd} 5-end and 3′-end primer pair, respectively. The PCR products were isolated, purified using the MinElute gel extraction kit, subcloned into an E. coli vector (pCR2.1 TOPO) and transformed into E. coli TOP10 cells. Several independent clones were cultured and used to prepare plasmid DNA. Subsequent sequence analysis confirmed that the nucleotide sequence contained the missing 5′-end of MTSP20 including part of the 5′-UTR.

Gene Expression Profile of MTSP20 in Normal Tissues, Tumor Tissues, Endothelial Cells and Tumor Cell Lines

To obtain information regarding the gene expression profile of the MTSP20 transcript, the MTSP20 cDNA fragment (˜400 bp) obtained from human lung tissue was used to probe a dot blot composed of RNA extracted from 76 different human tissues (Human Multiple Tissue Expression (MTE) Array; Clontech, Palo Alto, Calif.; catalog no. 7775-1; http://www.clontech.com). The results indicate that MTSP20 is expressed in many tissues. The MTSP20 transcript is found in liver, lymph node, cerebellum, pancreas, prostate, uterus, testis, glands (adrenal, thyroid and salivary), thymus, kidney and spleen. Lower transcript level is found in lung, placenta, bladder, ovary, digestive system, circulatory system and parts of the brain.

To compare the expression profile of MTSP20 transcript in a range of normal human and matched tumor tissues, a matched tumor/normal expression array (catalog number 7841-1; http://www.clontech.com) composed of 241 paired cDNA samples from individual patients was used. The membrane was hybridized with the same ³²P-labeled cDNA fragment as described before. Results show that the MTSP20 transcript is expressed at a low level in a number of normal tissues including breast, prostate, cervix, uterus, ovary, colon, lung, small intestine, stomach, kidney, pancreas, thyroid and rectum, but is not differentially expressed in any of the matched tumors. MTSP20 is also expressed in certain tumor cell lines including lung carcinoma (A519), colorectal carcinoma (SW480), lymphoma (Raji and Daudi), cervical carcinoma (HeLaS3) and leukemia (HL-60, K-562 and MOLT-4) cell lines.

Reverse Transcription-PCR Using Two Gene-Specific Primers

RT-PCR using 2 gene-specific primers (SEQ ID Nos. 9 and 10): 5′-GAAGCAGCTCATCCTGCATGGAGCCTACAC-3′ for the sense primer and 5′-GGCCAGGAACCATGTGCCCCTCACCTC-3′ for the anti-sense primer) showed the presence of MTSP20 in normal liver, normal lymph node, breast carcinoma cell line (MDA-MB-231) and in prostate carcinoma cell lines (LNCaP, PC-3 and CWR22R). This primer set amplifies a ˜380-bp cDNA fragment of MTSP20.

Using the same set of primers, several endothelial cell cDNA libraries were prepared from polyA⁺RNA purified from primary endothelial cell cultures (see, e.g., Clonetics; http://www.cambrex.com) and screened for the presence of the MTSP20 cDNA. Human aortic endothelial cells, human iliac artery endothelial cells, human lung microvascular endothelial cells and human umbilical cord endothelial cells all showed the presence of similar levels of MTSP20 cDNA. Human uterus microvascular endothelial cells also showed the presence of the MTSP20 cDNA at a lower level. To confirm the expression of MTSP20 by endothelial cells, another set of primers was used for PCR amplification: 5′-AAGCCTCAGGAGGGCAACACAGTCCCTG-3′ (SEQ ID No. 23) for the sense primer and 5′-ACAGCTGGGCAGCTCACCCACAGCACTGG-3′ (SEQ ID No. 24) for the antisense primer. This primer set amplifies a larger, ˜1.5 kbp fragment of MTSP20 cDNA. PCR reactions using as templates cDNA libraries prepared from human umbilical cord endothelial cells, human iliac artery endothelial cells, human aortic endothelial cells, human lung microvascular endothelial cells and human dermal microvascular endothelial cells produced the expected MTSP20 cDNA fragment.

Domain Structures of MTSP20 and Homology to Other Serine Proteases

Sequence and protein domain prediction analyses (see, e.g., http://smart.embl-heidelberg.de) of the translated MTSP20 protease domain sequence indicated that MTSP20 has a signal peptide (aa 1 to aa 24), a transmembrane domain (aa 82 to aa 99) and two potential N-glycosylation sites ( . . . N₇₇QS . . . and . . . N₆₂₁IS . . . ). MTSP20 has two trypsin-like serine protease domains in tandem, hereafter referred to as MTSP20-PD1 (aa 113 to aa 343) and MTSP20-PD2 (aa 375 to aa 596). Both protease domains are characterized by the presence of a protease activation cleavage site at the beginning of the domain and the catalytic triad residues (histidine, aspartate and serine) in 3 highly-conserved regions of the catalytic domain.

MTSP20-PD1 has the following features: a protease cleavage site ( . . . K₁₁₂↓P₁₁₃QEGN . . . , where ↓ indicates the cleavage site); catalytic triad residues (H₁₅₂, D₂₀₃ and S₂₉₉); the following cysteine pairings within the protease domain (C₁₃₇-C₁₅₃, C₂₃₃-C₃₀₅, C₂₆₄-C₂₈₄ and C₂₉₅-C₃₂₄); an unpaired cysteine (C₂₁₉) in the protease domain is predicted to pair with C₁₀₃.

MTSP20-PD2 has the following features: the protease cleavage site ( . . . R₃₇₅↓T₃₇₆AGPQ . . . ); catalytic triad residues (H₄₁₆, D₄₅₇ and S₅₅₃); the following cysteine pairings within the protease domain (C₄₀₁-C₄₁₇, C₅₁₉-C₅₃₉ and C₅₄₉-C₅₇₇); the unpaired cysteine (C₄₇₇) in the protease domain is predicted to pair with C₃₇₁.

Alignment (blastp; http://www.ncbi.nim.nih.gov/BLAST) of MTSP20-PD1 to Protein Sequences Deposited in the NCBI Protein Database

(http://www.ncbi.nlm.nih.gov/blast/html/blastcgihelp.html#protein_databa ses) showed the following homology: marapsin (35% identity; accession number NP_{—}114154.1); prostasin (33% identity); prostamin (31% identity; accession number AB030036); matriptase (31% identity, accession number NP_{—}068813); serine protease PRSS22 (32% identity, accession number NM_{—}022119); MTSP6/TADG12 (31%) and hepsin (32% identity, accession number NM_{—}002151). Alignment of the protease domain sequence of MTSP20-PD2 with that of gamma-II tryptase (accession number AF195508) and transmembrane tryptase (accession number XP_{—}008123) showed 37% and 36% identity, respectively. MTSP20-PD2 also shared homology to several other serine proteases including DESC1 (33% identity); MTSP6/TAGD12 (33% identity); prostasin (33% identity); prostamin (33% identity); matriptase (32% identity); airway trypsin-like protease (31% identity; accession number NP_{—}004253) and coagulation factor XI (30% identity, accession number M20218). Blast search of the coding region of MTSP20 against the sequences found in the Incyte database showed an apparent full-length clone (959903.FL1) that had a 1,661-bp insert. Clustal W alignment (using MacVector version 6.5.3; http://www.accelrys.com/products/macvector/index.html) of the nucleic acid and protein sequences of MTSP20 to the sequences derived from the Incyte clone showed 59% and 40% identity, respectively.

Sequence Analysis

MTSP20 cDNA and protein sequences were analyzed using MacVector (version 6.5.3; http://www.accelrys.com/products/macvector/index.html. The full-length cDNA encoding MTSP20 is 2,066 bp long. The coding region is 1,929 bp long which translates to a 642-amino acid protein as follows (see SEQ ID Nos. 15 and 16). MTSP20 cDNA and translated protein sequences: Sequence Range: 1 to 2066

MTSP20 cDNA and translated protein sequences
Sequence Range: 1 to 2066

```
          10  20  30  40  50  60
AGCACGGCCGGCCAATCGCCGAGTCAGAGGGCCAGGAGGGGCGCGGCCATTCGCCGCCCG
TCGTGCCGGCCGGTTAGCGGCTCAGTCTCCCGGTCCTCCCCGCGCCGGTAAGCGGCGGGC
                70  60  90  100  110  120
GCCCCTGCTCCGTGGCTGGTTTTCTCCGCGGGCGCCTCGGGCGGAACCTGGAGATAATGG
CGGGGACGAGGCACCGACCAAAAGAGGCGCCCGCGGAGCCCGCCTTGGACCTCTATTACC
                                                                                                                  M
              130  140  150  160  170  180
GCAGCACCTGGGGGAGCCCTGGCTGGGTGCGGCTCGCTCTTTGCCTGACGGGCTTAGTGC
CGTCGTGGACCCCCTCGGGACCGACCCACGCCGAGCGAGAAACGGACTGCCCGAATCACG
G  S  T  W  G  S  P  G  W  V  R  L  A  L  C  L  T  G  L  V
              190  200  210  220  230  240
TCTCGCTCTACGCGCTGCACGTGAAGGCGGCGCGCGCCCGGGACCGGGATTACCGCGCGC
AGAGCGAGATGCGCGACGTGCACTTCCGCCGCGCGCGGGCCCTGGCCCTAATGGCGCGCG
L  S  L  Y  A  L  H  V  K  A  A  R  A  R  D  R  D  Y  R  A
              250  260  270  280  290  300
TCTGCGACGTGGGCACCGCCATCAGCTGTTCGCGCGTCTTCTCCTCCAGGTGGGGCAGGG
AGACGCTGCACCCGTGGCGGTAGTCGACAAGCGCGCAGAAGAGGAGGTCCACCCCGTCCC
L  C  D  V  G  T  A  I  S  C  S  R  V  F  S  S  R  W  G  R
              310  320  330  340  350  360
GTTTCGGGCTGGTGGAGCATGTGCTGGGACAGGACAGCATCCTCAATCAATCCAACAGCA
CAAAGCCCGACCACCTCGTACACGACCCTGTCCTGTCGTAGGAGTTAGTTAGGTTGTCGT
G  F  G  L  V  E  H  V  L  G  Q  D  S  I  L  N  Q  S  N  S
              370  380  390  400  410  420
TATTCGGTTGCATCTTCTACACACTACAGCTATTGTTAGGTCTTCAAGCCGCTCAGCGTG
ATAAGCCAACGTAGAAGATGTGTGATGTCGATAACAATCCAGAAGTTCGGCGAGTCGCAC
I  F  G  C  I  F  Y  T  L  Q  L  L  L  G  L  Q  A  A  Q  R
              430  440  450  460  470  480
CCTGTGGACAGCGTGGCCCCGGCCCCCCCAAGCCTCAGGAGGGCAACACAGTCCCTGGCG
GGACACCTGTCGCACCGGGGCCGGGGGGGTTCGGAGTCCTCCCGTTGTGTCAGGGACCGC
A  C  G  Q  R  G  P  G  P  P  K  P  Q  E  G  N  T  V  P  G
              490  500  510  520  530  540
AGTGGCCCTGGCAGGCCAGTGTGAGGAGGCAAGGAGCCCACATCTGCAGCGGCTCCCTGG
TCACCGGGACCGTCCGGTCACACTCCTCCGTTCCTCGGGTGTAGACGTCGCCGAGGGACC
E  W  P  W  Q  A  S  V  R  R  Q  G  A  H  I  C  S  G  S  L
              550  560  570  580  590  600
TGGCAGACACCTGGGTCCTCACTGCTGCCCACTGCTTTGAAAAGGCAGCAGCAACAGAAC
ACCGTCTGTGGACCCAGGAGTGACGACGGGTGACGAAACTTTTCCGTCGTCGTTGTCTTG
V  A  D  T  W  V  L  T  A  A  H  C  F  E  K  A  A  A  T  E
              610  620  630  640  650  660
TGAATTCCTGGTCAGTGGTCCTGGGTTCTCTGCAGCGTGAGGGACTCAGCCCTGGGGCCG
ACTTAAGGACCAGTCACCAGGACCCAAGAGACGTCGCACTCCCTGAGTCGGGACCCCGGC
L  N  S  W  S  V  V  L  G  S  L  Q  R  E  G  L  S  P  G  A
              670  680  690  700  710  720
AAGAGGTGGGGGTGGCTGCCCTGCAGTTGCCCAGGGCCTATAACCACTACAGCCAGGGCT
TTCTCCACCCCCACCGACGGGACGTCAACGGGTCCCGGATATTGGTGATGTCGGTCCCGA
E  E  V  G  V  A  A  L  Q  L  P  R  A  Y  N  H  Y  S  Q  G
              730  740  750  760  770  780
CAGACCTGGCCCTGCTGCAGCTCGCCCACCCCACGACCCACACACCCCTCTGCCTGCCCC
GTCTGGACCGGGACGACGTCGAGCGGGTGGGGTGCTGGGTGTGTGGGGAGACGGACGGGG
S  D  L  A  L  L  Q  L  A  H  P  T  T  H  T  P  L  C  L  P
              790  800  810  820  830  840
AGCCCGCCCATCGCTTCCCCTTTGGAGCCTCCTGCTGGGCCACTGGCTGGGATCAGGACA
TCGGGCGGGTAGCGAAGGGGAAACCTCGGAGGACGACCCGGTGACCGACCCTAGTCCTGT
Q  P  A  H  R  F  P  F  G  A  S  C  W  A  T  G  W  D  Q  D
              850  860  870  880  890  900
CCAGTGATGCTCCTGGGACCCTACGCAATCTGCGCCTGCGTCTCATCAGTCGCCCCACAT
GGTCACTACGAGGACCCTGGGATGCGTTAGACGCGGACGCAGAGTAGTCAGCGGGGTGTA
T  S  D  A  P  G  T  L  R  N  L  R  L  R  L  I  S  R  P  T
              910  920  930  940  950  960
GTAACTGTATCTACAACCAGCTGCACCAGCGACACCTGTCCAACCCGGCCCGGCCTGGGA
CATTGACATAGATGTTGGTCGACGTGGTCGCTGTGGACAGGTTGGGCCGGGCCGGACCCT
C  N  C  I  Y  N  Q  L  H  Q  R  H  L  S  N  P  A  R  P  G
              970  980  990  1000  1010  1020
TGCTATGTGGGGGCCCCCAGCCTGGGGTGCAGGGCCCCTGTCAGGGAGATTCCGGGGGCC
ACGATACACCCCCGGGGGTCGGACCCCACGTCCCGGGGACAGTCCCTCTAAGGCCCCCGG
M  L  C  G  G  P  Q  P  G  V  Q  G  P  C  Q  G  D  S  G  G
            1030  1040  1050  1060  1070  1080
CTGTGCTGTGCCTCGAGCCTGACGGACACTGGGTTCAGGCTGGCATCATCAGCTTTGCAT
GACACGACACGGAGCTCGGACTGCCTGTGACCCAAGTCCGACCGTAGTAGTCGAAACGTA
P  V  L  C  L  E  P  D  G  H  W  V  Q  A  G  I  I  S  F  A
            1090  1100  1110  1120  1130  1140
CAAGCTGTGCCCAGGAGGACGCTCCTGTGCTGCTGACCAACACAGCTGCTCACAGTTCCT
GTTCGACACGGGTCCTCCTGCGAGGACACGACGACTGGTTGTGTCGACGAGTGTCAAGGA
S  S  C  A  Q  E  D  A  P  V  L  L  T  N  T  A  A  H  S  S
            1150  1160  1170  1180  1190  1200
GGCTGCAGGCTCGAGTTCAGGGGGCAGCTTTCCTGGCCCAGAGCCCAGAGACCCCGGAGA
CCGACGTCCGAGCTCAAGTCCCCCGTCGAAAGGACCGGGTCTCGGGTCTCTGGGGCCTCT
W  L  Q  A  R  V  Q  G  A  A  F  L  A  Q  S  P  E  T  P  E
            1210  1220  1230  1240  1250  1260
TGAGTGATGAGGACAGCTGTGTAGCCTGTGGATCCTTGAGGACAGCAGGTCCCCAGGCAG
ACTCACTACTCCTGTCGACACATCGGACACCTAGGAACTCCTGTCGTCCAGGGGTCCGTC
M  S  D  E  D  S  C  V  A  C  G  S  L  R  T  A  G  P  Q  A
            1270  1280  1290  1300  1310  1320
GAGCACCCTCCCCATGGCCCTGGGAGGCCAGGCTGATGCACCAGGGACAGCTGGCCTGTG
CTCGTGGGAGGGGTACCGGGACCCTCCGGTCCGACTACGTGGTCCCTGTCGACCGGACAC
G  A  P  S  P  W  P  W  E  A  R  L  M  H  Q  G  Q  L  A  C
            1330  1340  1350  1360  1370  1380
GCGGAGCCCTGGTGTCAGAGGAGGCGGTGCTAACTGCTGCCCACTGCTTCATTGGGCGCC
CGCCTCGGGACCACAGTCTCCTCCGCCACGATTGACGACGGGTGACGAAGTAACCCGCGG
G  G  A  L  V  S  E  E  A  V  L  T  A  A  H  C  F  I  G  R
            1390  1400  1410  1420  1430  1440
AGGCCCCAGAGGAATGGAGCGTAGGGCTGGGGACCAGACCGGAGGAGTGGGGCCTGAAGC
TCCGGGGTCTCCTTACCTCGCATCCCGACCCCTGGTCTGGCCTCCTCACCCCGGACTTCG
Q  A  P  E  E  W  S  V  G  L  G  T  R  P  E  E  W  G  L  K
            1450  1460  1470  1480  1490  1500
AGCTCATCCTGCATGGAGCCTACACCCACCCTGAGGGGGGCTACGACATGGCCCTCCTGC
TCGAGTAGGACGTACCTCGGATGTGGGTGGGACTCCCCCCGATGCTGTACCGGGAGGACG
Q  L  I  L  H  G  A  Y  T  H  P  E  G  G  Y  D  M  A  L  L
            1510  1520  1530  1540  1550  1560
TGCTGGCCCAGCCTGTGACACTGGGAGCCAGCCTGCGGCCCCTCTGCCTGCCCTATCCTG
ACGACCGGGTCGGACACTGTGACCCTCGGTCGGACGCCGGGGAGACGGACGGGATAGGAC
L  L  A  Q  P  V  T  L  G  A  S  L  R  P  L  C  L  P  Y  P
            1570  1580  1590  1600  1610  1620
ACCACCACCTGCCTGATGGGGAGCGTGGCTGGGTTCTGGGACGGGCCCGCCCAGGAGCAG
TGGTGGTGGACGGACTACCCCTCGCACCGACCCAAGACCCTGCCCGGGCGGGTCCTCGTC
D  H  H  L  P  D  G  E  R  G  W  V  L  G  R  A  R  P  G  A
            1630  1640  1650  1660  1670  1680
GCATCAGCTCCCTCCAGACAGTGCCCGTGACCCTCCTGGGGCCTAGGGCCTGCAGCCGGC
CGTAGTCGAGGGAGGTCTGTCACGGGCACTGGGAGGACCCCGGATCCCGGACGTCGGCCG
G  I  S  S  L  Q  T  V  P  V  T  L  L  G  P  R  A  C  S  R
            1690  1700  1710  1720  1730  1740
TGCATGCAGCTCCTGGGGGTGATGGCAGCCCTATTCTGCCGGGGATGGTGTGTACCAGTG
ACGTACGTCGAGGACCCCCACTACCGTCGGGATAAGACGGCCCCTACCACACATGGTCAC
L  H  A  A  P  G  G  D  G  S  P  I  L  P  G  M  V  C  T  S
            1750  1760  1770  1780  1790  1800
CTGTGGGTGAGCTGCCCAGCTGTGAGGGCCTGTCTGGGGCACCACTGGTGCATGAGGTGA
GACACCCACTCGACGGGTCGACACTCCCGGACAGACCCCGTGGTGACCACGTACTCCACT
A  V  G  E  L  P  S  C  E  G  L  S  G  A  P  L  V  H  E  V
            1810  1820  1830  1840  1850  1860
GGGGCACATGGTTCCTGGCCGGGCTGCACAGCTTCGGAGATGCTTGCCAAGGCCCCGCCA
CCCCGTGTACCAAGGACCGGCCCGACGTGTCGAAGCCTCTACGAACGGTTCCGGGGCGGT
R  G  T  W  F  L  A  G  L  H  S  F  G  D  A  C  Q  G  P  A
            1870  1880  1890  1900  1910  1920
GGCCGGCGGTCTTCACCGCGCTCCCTGCCTATGAGGACTGGGTCAGCAGTTTGGACTGGC
CCGGCCGCCAGAAGTGGCGCGAGGGACGGATACTCCTGACCCAGTCGTCAAACCTGACCG
R  P  A  V  F  T  A  P  A  Y  E  D  W  V  S  S  L  D  W
            1930  1940  1950  1960  1970  1980
AGGTCTACTTCGCCGAGGAACCAGAGCCCGAGGCTGAGCCTGGAAGCTGCCTGGCCAACA
TCCAGATGAAGCGGCTCCTTGGTCTCGGGCTCCGACTCGGACCTTCGACGGACCGGTTGT
Q  V  Y  F  A  E  E  P  E  P  E  A  E  P  G  S  C  L  A  N
            1990  2000  2010  2020  2030  2040
TAAGTATGTGGCCCCGGGGCCTCCTGCCAAACCCTGCCTCTCCAGGACCCTTCTCTCTCC
ATTCATACACCGGGGCCCCGGAGGACGGTTTGGGACGGAGAGGTCCTGGGAAGAGAGAGG
I  S  M  W  P  R  G  L  L  P  N  P  A  S  P  G  P  F  S  L
            2050  2060
AGTGAGAACGTGCCGTTCTAACCCAC
TCACTCTTGCACGGCAAGATTGGGTG
Q  *
MTSP20 protein sequence
Sequence Range: 1 to 643
                10  20  30  40  50  60
MGSTWGSPGWVRLALCLTGLVLSLYALHVKAARARDRDYRALCDVGTAISCSRVFSSRWG
                70  80  90  100  110  120
RGFGLVEHVLGQDSILNQSNSIFGCIFYTLQLLLGLQAAQRACGQRGPGPPKPQEGNTVP
              130  140  150  160  170  180
GEWPWQASVRRQGAHICSGSLVADTWVLTAAHCFEKAAATELNSWSVVLGSLQREGLSPG
              190  200  210  220  230  240
AEEVGVAALQLPRAYNHYSQGSDLALLQLAHPTTHTPLCLPQPAHRFPFGASCWATGWDQ
              250  260  270  280  290  300
DTSDAPGTLRNLRLRLISRPTCNCIYNQLHQRHLSNPARPGMLCGGPQPGVQGPCQGDSG
              310  320  330  340  350  360
GPVLCLEPDGHWVQAGIISFASSCAQEDAPVLLTNTAAHSSWLQARVQGAAFLAQSPETP
              370  380  390  400  410  420
EMSDEDSCVACGSLRTAGPQAGAPSPWPWEARLMHQGQLACGGALVSEEAVLTAAHCFIG
              430  440  450  460  470  480
RQAPEEWSVGLGTRPEEWGLKQLILHGAYTHPEGGYDMALLLLAQPVTLGASLRPLCLPY
              490  500  510  520  530  540
PDHHLPDGERGWVLGRARPGAGISSLQTVPVTLLGPRACSRLHAAPGGDGSPILPGMVCT
              550  560  570  580  590  600
SAVGELPSCEGLSGAPLVHEVRGTWFLAGLHSFGDACQGPARPAVFTALPAYEDWVSSLD
              610  620  630  640
WQVYFAEEPEPEAEPGSCLANISMWPRGLLPNPASPGPFSLQ*
```

### EXAMPLE 2

Expression of the Protease MTSP Domains

Nucleic acid encoding the MTSP20 and protease domain thereof can be cloned into a derivative of the Pichia pastoris vector pPIC9K (available from Invitrogen; see SEQ ID NO. 13). Plasmid pPIC9k features include the 5′ AOX1 promoter fragment at 1-948; 5′ AOX1 primer site at 855-875; alpha-factor secretion signal(s) at 949-1218; alpha-factor primer site at 1152-1172; multiple cloning site at 1192-i 241; 3′ AOX1 primer site at 1327-1347; 3′ AOX1 transcription termination region at 1253-1586; HIS4 ORF at 4514-1980; kanamycin resistance gene at 5743-4928; 3′ AOX1 fragment at 6122-6879; ColE1 origin at 7961-7288; and the ampicillin resistance gene at 8966-8106. The plasmid used herein is derived from pPIC9K by eliminating the Xhol site in the kanamycin resistance gene and the resulting vector is herein designated pPIC9KX. Expression in Pichia can be performed using known methods (see, e.g., Zhang et al. (2000) Biotechnology and Bioengineering 70: No 1 Oct. 5, 2000).

Mutagenesis of a Protease Domain of MTSP20 for Expression in Pichia

Each or both protease domains can be mutated so that the unpaired Cys is replaced with another amino acid, such as Ser, to decrease or prevent aggregation of the expressed polypeptide.

### EXAMPLE 3

Assays for Identification of Candidate Compounds that Modulate that Activity of an MTSP

Assay for Screening MTSP20 Inhibitors

A protease domain (or plurality thereof) of MTSP20 expressed in Pichia pastoris is assayed for inhibition by various compounds as follows in Costar 96 well tissue culture plates (Corning N.Y.). Approximately 1-10 nM MTSP20 is added without inhibitor, or with 100000 nM inhibitor and 71:6 dilutions to 1X direct buffer (29.2 mM Tris, pH 8.4, 29.2 mM Imidazole, 217 mM NaCl (100 μl final volume)), and allowed to incubate at room temperature for 30 minutes. 400 μM substrate Spectrozyme t-PA (American Diagnostica, Greenwich, Conn.) is added and reaction is monitored in a SpectraMAX Plus microplate reader (Molecular Devices, Sunnyvale Calif.) by following change in absorbance at 405 nm for 20 minutes at 37° C. Spectrozyme UK can also be used as the substrate in this assay.

Identification of Substrates

Other substrates for use in the assays can be identified empirically by testing substrates. The following list of substrates are exemplary of those that can be tested.

| Substrate name | Structure |
|---|---|
| S 2366 | pyroGlu-Pro-Arg-pNA.HCl |
| spectrozyme t-PA | CH₃SO₂-D-HHT-Gly-Arg-pNA.AcOH |
| N-p-tosyl-Gly-Pro-Arg-pNA | N-p-tosyl-Gly-Pro-Arg-pNA |
| Benzoyl-Val-Gly-Arg-pNA | Benzoyl-Val-Gly-Arg-pNA |
| Pefachrome t-PA | CH₃SO₂-D-HHT-Gly-Arg-pNA |
| S 2765 | N-α-Z-D-Arg-Gly-Arg-pNA.2HCl |
| S 2444 | pyroGlu-Gly-Arg-pNA.HCl |
| S 2288 | H-D-IIe-Pro-Arg-pNA.2HCl |
| spectrozyme UK | Cbo-L-(γ)Glu(α-t-BuO)-Gly-Arg-pNA.2AcOH |
| S 2302 | H-D-Pro-Phe-Arg-pNA.2HCl |
| S 2266 | H-D-Val-Leu-Arg-pNA.2HCl |
| S 2222 | Bz-IIe-Glu(g-OR)-Gly-Arg-pNA.HCl |
| | R = H(50%) and R = CH₃(50%) |
| Chromozyme PK | Benzoyl-Pro-Phe-Arg-pNA |
| S 2238 | H-D-Phe-Pip-Arg-pNA.2HCl |
| S 2251 | H-D-Val-Leu-Lys-pNA.2HCl |
| Spectrozyme PI | H-D-Nle-HHT-Lys-pNA.2AcOH |
| | Pyr-Arg-Thr-Lys-Arg-AMC |
| | H-Arg-Gln-Arg-Arg-AMC |
| | Boc-Gln-Gly-Arg-AMC |
| | Z-Arg-Arg-AMC |
| Spectrozyme THE | H-D-HHT-Ala-Arg-pNA.2AcOH |
| Spectrozyme fXIIa | H-D-CHT-Gly-Arg-pNA.2AcOH |
| | CVS 2081-6 (MeSO₂-dPhe-Pro-Arg-pNA) |
| | Pefachrome fVIIa (CH₃SO₂-D-CHA-But-Arg-pNA) |

pNA = para-nitranilide (chromogenic)
AMC = amino methyl coumarin (fluorescent)

If none of the above substrates are cleaved, a coupled assay can be used. Briefly, a coupled assay tests the ability of the protease to activate an enzyme, such as plasminogen and trypsinogen. To perform these assays, a single chain protease domain (or plurality thereof) is incubated with a zymogen, such as plasminogen or trypsinogen, in the presence of the a known substrate, such as a chromogenic substrate (i.e., S2366, see TABLE above) for the zymogen. If the single chain activates the zymogen, the activated enzyme, such as plasmin and trypsin, will degrade the substrate therefor (i.e., S2366).

### EXAMPLE 4

Other Assays

These assays are described with reference to MTSP1, but such assays can be readily adapted for use with MTSP20.

Amidolytic Assay for Determining Inhibition of Serine

Protease Activity of Matriptase or MTSP1

The ability of test compounds to act as inhibitors of rMAP catalytic activity is assessed by determining the inhibitor-induced inhibition of amidolytic activity by the MAP, as measured by IC₅₀ values. The assay buffer is HBSA (10 mM Hepes, 150 mM sodium chloride, pH 7.4, 0.1% bovine serum albumin). All reagents are from Sigma Chemical Co. (St. Louis, Mo.), unless otherwise indicated.

Two IC₅₀ assays (a) one at either 30-minutes or 60-minutes (a 30-minute or a 60-minute preincubation of test compound and enzyme) and (b) one at 0-minutes (no preincubation of test compound and enzyme) is conducted. For the IC₅₀ assay at either 30-minutes or 60-minutes, the following reagents are combined in appropriate wells of a Corning microtiter plate: 50 microliters of HBSA, 50 microliters of the test compound, diluted (covering a broad concentration range) in HBSA (or HBSA alone for uninhibited velocity measurement), and 50 microliters of the rMAP (Corvas International) diluted in buffer, yielding a final enzyme concentration of 250 pM as determined by active site filtration. Following either a 30-minute or a 60-minute incubation at ambient temperature, the assay is initiated by the addition of 50 microliters of the substrate S-2765 (N-a-Benzyloxycarbonyl-D-arginyl-L-glycyl-L-arginine-p-nitroaniline dihydrochloride; DiaPharma Group, Inc.; Franklin, Ohio) to each well, yielding a final assay volume of 200 microliters and a final substrate concentration of 100 μM (about 4-times Kₘ) Before addition to the assay mixture, S-2765 is reconstituted in deionized water and diluted in HBSA. For the IC₅₀ assay at 0 minutes; the same reagents are combined: 50 microliters of HBSA, 50 microliters of the test compound, diluted (covering the identical concentration range) in HBSA (or HBSA alone for uninhibited velocity measurement), and 50 microliters of the substrate S-2765. The assay is initiated by the addition of 50 microliters of rMAP. The final concentrations of all components are identical in both IC₅₀ assays (at 30- or 60- and 0-minute).

The initial velocity of chromogenic substrate hydrolysis is measured in both assays by the change of absorbance at 405 nM using a Thermo Max® Kinetic Microplate Reader (Molecular Devices) over a 5 minute period, in which less than 5% of the added substrate is used. The concentration of added inhibitor, which caused a 50% decrease in the initial rate of hydrolysis is defined as the respective IC₅₀ value in each of the two assays (30- or 60-minutes and 0-minute).

In vitro Enzyme Assays for Specificity Determination

The ability of compounds to act as a selective inhibitor of matriptase activity is assessed by determining the concentration of test compound that inhibits the activity of matriptase by 50%, (IC₅₀) as described in the above Example, and comparing IC₅₀ value for matriptase to that determined for all or some of the following serine proteases: thrombin, recombinant tissue plasminogen activator (rt-PA), plasmin, activated protein C, chymotrypsin, factor Xa and trypsin.

The buffer used for all assays is HBSA (10 mM HEPES, pH 7.5, 150 mM sodium chloride, 0.1% bovine serum albumin).

The assay for IC₅₀ determinations is conducted by combining in appropriate wells of a Corning microtiter plate, 50 microliters of HBSA, 50 microliters of the test compound at a specified concentration (covering a broad concentration range) diluted in HBSA (or HBSA alone for V₀ (uninhibited velocity) measurement), and 50 microliters of the enzyme diluted in HBSA. Following a 30 minute incubation at ambient temperature, 50 microliters of the substrate at the concentrations specified below are added to the wells, yielding a final total volume of 200 microliters. The initial velocity of chromogenic substrate hydrolysis is measured by the change in absorbance at 405 nm using a Thermo Max® Kinetic Microplate Reader over a 5 minute period in which less than 5% of the added substrate is used. The concentration of added inhibitor which caused a 50% decrease in the initial rate of hydrolysis is defined as the IC₅₀ value.

Thrombin (fIIa) Assay

Enzyme activity is determined using the chromogenic substrate, Pefachrome t-PA (CH₃SO₂-D-hexahydrotyrosine-glycyl-L-Arginine-p-nitroaniline, obtained from Pentapharm Ltd.). The substrate is reconstituted in deionized water prior to use. Purified human a-thrombin is obtained from Enzyme Research Laboratories, Inc. The buffer used for all assays is HBSA (10 mM HEPES, pH 7.5, 150 mM sodium chloride, 0.1% bovine serum albumin).

IC₅₀ determinations are conducted where HBSA (50 μL), a-thrombin (50 μl) (the final enzyme concentration is 0.5 nM) and inhibitor (50 μl) (covering a broad concentration range), are combined in appropriate wells and incubated for 30 minutes at room temperature prior to the addition of substrate Pefachrome-t-PA (50 μl) (the final substrate concentration is 250 μM, about 5 times Km). The initial velocity of Pefachrome t-PA hydrolysis is measured by the change in absorbance at 405 nm using a Thermo Max® Kinetic Microplate Reader over a 5 minute period in which less than 5% of the added substrate is used. The concentration of added inhibitor which caused a 50% decrease in the initial rate of hydrolysis is defined as the IC₅₀ value.

Factor Xa

Factor Xa catalytic activity is determined using the chromogenic substrate S-2765 (N-benzyloxycarbonyl-D-arginine-L-glycine-L-arginine-p-nitroaniline), obtained from DiaPharma Group (Franklin, Ohio). All substrates are reconstituted in deionized water prior to use. The final concentration of S-2765 is 250 μM (about 5-times Km). Purified human Factor X is obtained from Enzyme Research Laboratories, Inc. (South Bend, Ind.) and Factor Xa (FXa) is activated and prepared from it as described (Bock, P. E., Craig, P. A., Olson, S. T., and Singh, P. Arch. Biochem. Biophys. 273:375-388 (1989)). The enzyme is diluted into HBSA prior to assay in which the final concentration is 0.25 nM.

Recombinant Tissue Plasminogen Activator (rt-PA) Assay

rt-PA catalytic activity is determined using the substrate, Pefachrome t-PA (CH₃SO₂-D-hexahydrotyrosine-glycyl-L-arginine-p-nitroaniline, obtained from Pentapharm Ltd.). The substrate is made up in deionized water followed by dilution in HBSA prior to the assay in which the final concentration is 500 micromolar (about 3-times Km). Human rt-PA (Activase®) is obtained from Genentech Inc. The enzyme is reconstituted in deionized water and diluted into HBSA prior to the assay in which the final concentration is 1.0 nM.

Plasmin Assay

Plasmin catalytic activity is determined using the chromogenic substrate, S-2366 (L-pyroglutamyl-L-prolyl-L-arginine-p-nitroaniline hydrochloride), which is obtained from DiaPharma group. The substrate is made up in deionized water followed by dilution in HBSA prior to the assay in which the final concentration is 300 micromolar (about 2.5-times Km). Purified human plasmin is obtained from Enzyme Research Laboratories, Inc. The enzyme is diluted into HBSA prior to assay in which the final concentration is 1.0 nM.

Activated Protein C (aPC) Assay

aPC catalytic activity is determined using the chromogenic substrate, Pefachrome PC (delta-carbobenzloxy-D-lysine-L-prolyl-L-arginine-p-nitroaniline dihydrochloride, obtained from Pentapharm Ltd.). The substrate is made up in deionized water followed by dilution in HBSA prior to the assay in which the final concentration is 400 micromolar (about 3-times Km). Purified human aPC is obtained from Hematologic Technologies, Inc. The enzyme is diluted into HBSA prior to assay in which the final concentration is 1.0 nM.

Chymotrypsin Assay

Chymotrypsin catalytic activity is determined using the chromogenic substrate, S-2586 (methoxy-succinyl-L-arginine-L-prolyl-L-tyrosyl-p-nitroanilide), which is obtained from DiaPharma Group. The substrate is made up in deionized water followed by dilution in HBSA prior to the assay in which the final concentration is 100 micromolar (about 9-times Km). Purified (3X-crystallized; CDI) bovine pancreatic alpha-chymotrypsin is obtained from Worthington Biochemical Corp. The enzyme is reconstituted in deionized water and diluted into HBSA prior to assay in which the final concentration is 0.5 nM.

Trypsin Assay

Trypsin catalytic activity is determined using the chromogenic substrate, S-2222 (benzoyl-L-isoleucine-L-glutamic acid-[gamma-methyl ester]-L-arginine-p-nitroanilide), which is obtained from DiaPharma Group. The substrate is made up in deionized water followed by dilution in HBSA prior to the assay in which the final concentration is 250 micromolar (about 4-times Km). Purified (3X-crystallized; TRL3) bovine pancreatic trypsin is obtained from Worthington Biochemical Corp. The enzyme is reconstituted in deionized water and diluted into HBSA prior to assay in which the final concentration is 0.5 nM.

Since modifications will be apparent to those of skill in this art, it is intended that this invention be limited only by the scope of the appended claims.

What is claimed is:

1. A substantially purified single or multi-chain polypeptide, comprising a protease domain of a type-II membrane-type serine protease 20 (MTSP20) or a catalytically active portion thereof, wherein the polpeptide comprises two protease domains.

2. A substantially purified polypeptide that comprises an MTSP20 protease domain 2 (PD2) domain.

3. A substantially purified polypeptide, that consists essentially of an MTSP20 protease domain 1 (PD1) domain.

4. The polypeptide of claim 1 that is an activated two or three-chain polypeptide.

5. A polypeptide of claim 1 selected from the group consisting of:
a polypeptide that comprises a sequence of amino acids encoded by the sequence that includes at least about 60%, 70%, 80% or 90% amino acid sequence identity with the sequence of amino acids set forth in SEQ ID No. 6 or SEQ ID No. 16;
a polypeptide that comprises a sequence of amino acids encoded by the sequence that includes at least about 60%, 70%, 80% or 90% amino acid sequence identity with the sequence of amino acids set forth in SEQ ID No. 6 or SEQ ID No. 6;
a polypeptide that comprises a sequence of amino acids encoded by the sequence of nucleotides set forth in SEQ ID No. 5 or SEQ ID No. 15;
a polypeptide that comprises a sequence of amino acids encoded by a sequence of nucleotides that hybridizes along at least 70% of its full-length under conditions of high stringency to the sequence of nucleotides set forth as SEQ ID No. 5 or SEQ ID No. 15 or to a portion thereof that encodes at least one protease domain that includes amino acids 624-642 of SEQ ID No. 16;
a polypeptide that is encoded by a sequence of nucleotides that is a splice variant of the sequence of nucleotides that comprises the sequence set forth in SEQ ID No. 5 or SEQ ID No. 15.

6. The substantially purified polypeptide of claim 5, wherein the MTSP20 is a human polypeptide.

7. The substantially purified polypeptide of claim 3 that consists essentially of the PD2 of MTSP20 or a catalytically active portion of the the PD2 protease domain of MTSP20.

8. The substantially purified polypeptide of claim 1 that comprises the sequence of amino acids set forth as amino acids in SEQ ID No. 16.

9. A substantially purified polypeptide of claim 2 that consists essentially of the sequence of amino acids set forth as residues 376-624 in SEQ ID No. 16 or catalytically active fragments thereof.

10. The polypeptide of claim 9 that further includes one or more contiguous residues 625 through 642 of SEQ ID No. 16 following residue 624.

11. A substantially purified polypeptide of claim 3 that consists essentially of the sequence of amino acids set forth in SEQ ID No. 6 or amino acids 113-343 of SEQ ID No. 16 or catalytically active fragments of the sequence of amino acids set forth in SEQ ID No. 6 or of amino acids 113-343 of SEQ ID No. 16.

12. The substantially purified polypeptide of claim 1 that has at least about 60%, 70%, 80% or 90% sequence identity with a polypeptide that comprises the sequence of amino acids set forth as SEQ ID No. 16, wherein the substantially purified polypeptide is a protease.

13. A polypeptide of claim 5, wherein a protease domain portion is encoded by a nucleic acid molecule that hybridizes under conditions of at least low stringency along at least 70% of its full-length to a nucleic acid molecule comprising a sequence of nucleotides set forth in SEQ ID No. 5 or that encodes a catalytically active portion of a protease domain or that hybridizes under conditions of at least low stringency along at least 70% of its full-length to a nucleic acid molecule that encodes residues 376-642 of SEQ ID No. 16. or that encodes a catalytically active portion of a protease domain.

14. The polypeptide of claim 13, wherein the conditions of hybridization are high stringency conditions.

15. A polypeptide of claim 5 that is a mutein, wherein:
up to about 50% of the amino acids are replaced with another amino acid;
and the resulting polypeptide is a single chain or multi-chain polypeptide that has catalytic activity of at least 1%, 5% or 10% of the unmutated polypeptide.

16. The polypeptide of claim 15, wherein up to about 20% of the amino acids are replaced with another amino acid.

17. The polypeptide of claim 15, wherein the resulting polypeptide is a single chain or multi-chain polypeptide and has catalytic activity of at least 1% of the unmutated polypeptide.

18. The polypeptide of claim 15, wherein:
a cysteine in a protease domain is replaced with another amino acid; and the cysteine is one that is unpaired in a polypeptide that consists essentially of a protease domain.

19. The polypeptide of claim 18, wherein the replacing amino acid is a serine.

20. A nucleic acid molecule, comprising a sequence of nucleotides that encodes a polypeptide of claim 1.

21. The nucleic acid molecule of claim 20 that comprises a sequence of nucleotides selected from the group consisting of:
(a) a sequence of nucleotides set forth as nucleotides 1-642 in SEQ ID No. 16 or set forth in SEQ ID No. 5 or a portion thereof that encodes a proteolytically active polypeptide;
(b) a sequence of nucleotides that hybridizes under high stringency along its length or along at least about 70% of the full-length to the sequence of nucleotides in SEQ ID No. 5 or SEQ ID No. 15 or to a portion thereof that encodes a protease domain of an MTSP20;
(c) a sequence of nucleotides that encodes the polypeptide of SEQ ID No. 6 or of SEQ ID No. 16;
(d) a sequence of nucleotides that is a splice variant of (a), (b) or (c);
(e) a sequence of nucleotides that encodes a protease domain or a catalytically active portion thereof that includes a sequence of nucleotides having at least about 60%, 70%, 80%, 90% or 95% sequence identity with the sequence set forth in SEQ ID No. 5 or SEQ ID No. 15; and
(f) a sequence of nucleotides comprising degenerate codons of (a), (b), (c), (d) or (e).

22. An isolated nucleic molecule that encodes a mutein polypeptide of claim 15.

23. A vector comprising the nucleic acid molecule of claim 20.

24. The vector of claim 23 that is an expression vector.

25. The vector of claim 23 that is a eukaryotic vector.

26. The vector of claim 23 that includes a sequence of nucleotides that directs secretion of any polypeptide encoded by a sequence of nucleotides operatively linked thereto.

27. The vector of claim 26 that is a Pichia vector or an E. coli vector.

28. A cell, comprising the vector of claim 23.

29. The cell of claim 28 that is a prokaryotic cell.

30. The cell of claim 28 that is a eukaryotic cell.

31. The cell of claim 28 that is selected from among a bacterial cell, a yeast cell, a plant cell, an insect cell and an animal cell.

32. The cell of claim 28 that is a mammalian cell.

33. A recombinant non-human animal, wherein an endogenous gene that encodes a polypeptide of claim 1 has been deleted or inactivated by homologous recombination or insertional mutagenesis of a gene of the animal or an ancestor thereof.

34. A method for producing a polypeptide that contains a protease domain of an MTSP20 polypeptide, comprising:
culturing the cell of claim 28 under conditions whereby the encoded polypeptide is expressed by the cell; and
recovering the expressed polypeptide.

35. The method of claim 34, wherein the polypeptide is secreted into the culture medium.

36. The method of claim 34, wherein the cell is a Pichia cell.

37. An isolated cell that comprises a polypeptide of claim 1 expressed on its surface.

38. An antisense nucleic acid molecule that comprises at least 14 contiguous nucleotides or modified nucleotides that are complementary to a contiguous sequence of nucleotides encoding an MTSP20 of claim 5; or
comprises at least 16 contiguous nucleotides or modified nucleotides that are complementary to a contiguous sequence of nucleotides encoding the an MTSP20 polypeptide of claim 5; or
comprises at least 30 contiguous nucleotides or modified nucleotides that are complementary to a contiguous sequence of nucleotides encoding an MTSP20 polypeptide of claim 5.

39. The antisense molecule of claim 38 that includes a contiguous sequence of nucleotides that is the complement of a sequence of nucleotides in SEQ ID No. 5 or SEQ ID No. 15.

40. A double-stranded RNA (dsRNA) molecule that comprises at least about 21 contiguous nucleotides or modified nucleotides from a sequence of nucleotides encoding an MTSP20 of claim 5.

41. An antibody that specifically binds to the polpeptide of claim 1, but that does not bind to MTSP20 molecules encoded by splice variants lacking a full-lenth protease 2 domain.

42. An antibody that binds to the polpeptide of claim 1 with at least 10-fold greater affinity than to a polypeptide encoded by SEQ ID No. 17.

43. An antibody of claim 42 that binds with at least 100-fold greater affinity.

44. An antibody of claim 41 that inhibits an enzymatic activity of the polypeptide.

45. An antibody that specifically binds to the protease domain 2 (PD2) of an MTSP20 polypeptide.

46. The antibody of claim 41 that binds to a single-chain form of an MTSP20 polypeptide or a PD1 or PD2 protease domain thereof.

47. The antibody of claim 41 that is a monoclonal antibody.

48. The antibody of claim 41 that is a polyclonal antibody.

49. A conjugate, comprising:
a polypeptide of claim 1, and
a targeting agent linked to the polypeptide directly or via a linker.

50. The conjugate of claim 49, wherein the targeting agent permits
affinity isolation or purification of the conjugate;
attachment of the conjugate to a surface;
detection of the conjugate; or
targeted delivery to a selected tissue or cell.

51. A combination, comprising:
an agent or treatment that inhibits a catalytic activity of a polypeptide of claim 1; and
another treatment or agent selected from anti-tumor and anti-angiogenic treatments and agents.

52. The combination of claim 51, wherein the inhibitor and the anti-tumor and/or anti-angiogenic agent are formulated in a single pharmaceutical composition or each is formulated in separate pharmaceutical compositions.

53. The combination of claim 52, wherein the inhibitor is selected from antibodies and antisense oligonucleotides and double-stranded RNA (dsRNA).

54. A solid support comprising two or more polypeptides of claim 1 and/ or a polypeptide of SEQ ID No. 17 linked thereto either directly or via a linker.

55. The support of claim 54, wherein the polypeptides comprise an array.

56. The support of claim 55, wherein the polypeptides comprise a plurality of protease domains.

57. A solid support comprising two or more nucleic acid molecules of claim 20 or oligonucleotide portions thereof linked thereto either directly or via a linker, wherein the oligonucleotides contain at least 16 nucleotides.

58. The support of claim 57, wherein the nucleic acid molecules comprise an array.

59. The support of claim 57, wherein the nucleic acid molecules comprise a plurality of molecules that encode protease domains.

60. A method for identifying or screening compounds that modulate a protease activity of an MTSP20 polypeptide, comprising:
contacting a polypeptide of claim 1 or a polypeptide of SEQ ID No. 17 or a proteolytically active portion of the polypeptide with a substrate that is proteolytically cleaved by the polypeptide, and, either simultaneously, before or after, adding a test compound or plurality thereof;
measuring the amount of substrate cleaved in the presence of the test compound; and
selecting compounds that change the amount of substrate cleaved compared to a control, whereby compounds that modulate an activity of the polypeptide are identified.

61. The method of claim 60, wherein the test compounds are small molecules, peptides, peptidomimetics, nucleic acid molecules, natural products, antibodies or fragments thereof that modulate an activity of the polypeptide.

62. The method of claim 60, wherein a plurality of the test substances are screened simultaneously.

63. The method of claim 60, wherein the polypeptide is encoded by a sequence of nucleotides selected from the group consisting of:
(a) a sequence of nucleotides in SEQ ID No. 5 or SEQ ID No. 15 or a sequence of nucleotides that encodes the polyeptide of SEQ ID No. 17;
(b) a sequence of nucleotides that hybridizes under high stringency along its length or along at least about 70% of the full-length to the sequence of nucleotides in SEQ ID No. 5 or SEQ ID No. 15 or a sequence of nucleotides that encodes the polyeptide of SEQ ID No. 17;
(c) a sequence of nucleotides that encodes the polypeptide of SEQ ID No. 6 or SEQ ID No. 16 or SEQ ID No. 17;
(d) a sequence of nucleotides that is a splice variant of (a), (b) or (c);
(e) a sequence of nucleotides that encodes a protease domain or a catalytically active portion thereof that includes a sequence of nucleotides having at least about 60%, 70%, 80%, 90% or 95% sequence identity with the sequence set forth in SEQ ID No. 5 or 15; and
(f) a sequence of nucleotides comprising degenerate codons of (a), (b), (c), (d) or (e).

64. The method of claim 60, wherein the polypeptide is selected from the group consisting of:
a polypeptide that consists essentially of a sequence of amino acids set forth in SEQ ID No. 6 or as amino acids 113-343 of SEQ ID No. 16 or as amino acids 376-642 of SEQ ID No. 16;
a polypeptide that consists essentially of a sequence of amino acids encoded by a sequence of nucleotides that hybridizes under conditions of high stringency to the sequence of nucleotides in SEQ ID No. 5 or set forth as nucleotides 360-1052 or 1148-1952 in SEQ ID No. 15;
a polypeptide that comprises the sequence of amino acids set forth as residues 376-642 SEQ ID No. 16;
a polypeptide that comprises a sequence of amino acids having at least about 60% sequence identity with the sequence of amino acids in SEQ ID No. 6 or the sequence of amino acids in SEQ ID No. 16;
a polypeptide that is encoded by a sequence of nucleotides that is a splice variant of the sequence set forth in SEQ ID No. 15.

65. The method of claim 60, wherein the change in the amount of substrate cleaved is assessed by comparing the amount of substrate cleaved in the presence of the test compound with the amount of substrate cleaved in the absence of the test compound.

66. The method of claim 60, wherein a plurality of the polypeptides are linked to a solid support, either directly or via a linker.

67. The method of claim 66, wherein the polypeptides comprise an array.

68. A method of identifying a compound that specifically binds to a single-chain and/or multi-chain MTSP20 protease domain and/or to a single or multi-chain MTSP20 polypeptide and/or to a proteolytically active portion of a single or a multi-chain form of an MTSP20 polypeptide, comprising:
contacting an MTSP20 polypeptide of claim 1 or a polypeptide of SEQ ID No. 17 or a proteolytically active portion of the polypeptide with a test compound or plurality thereof under conditions conducive to binding thereof; and either:
a) identifying test compounds that specifically bind to the single chain and/or multi chain form of the polypeptide or to a proteolytically active portion of the single and/or multi chain form thereof, or
b) identifying test compounds that inhibit binding of a compound known to bind a single chain and/or multi chain form of the polypeptide or to a proteolytically active portion of the single and/or multi-chain form thereof, wherein the known compound is contacted with the polypeptide before, simultaneously with or after the test compound, wherein the multi-chain form is a two, three or four chain polypeptide.

69. The method of claim 68, wherein the polypeptide is linked either directly or indirectly via a linker to a solid support.

70. The method of claim 68, wherein the test compounds are small molecules, peptides, peptidomimetics, natural products, antibodies or fragments thereof.

71. The method of claim 68, wherein a plurality of the test substances are screened simultaneously.

72. The method of claim 71, wherein the polypeptides comprise an array.

73. The method of claim 68, wherein the polypeptide is encoded by a sequence of nucleotides selected from the group consisting of:
(a) a sequence of nucleotides in SEQ ID No. 5 or SEQ ID No. 15 or a sequence of nucleotides that encodes the polyeptide of SEQ ID No. 17;
(b) a sequence of nucleotides that hybridizes under high stringency along its length or along at least about 70% of the full-length to the sequence of nucleotides in SEQ ID No. 5 or SEQ ID No. 15 or a sequence of nucleotides that encodes the polyeptide of SEQ ID No. 17;
(c) a sequence of nucleotides that encodes the polypeptide of SEQ ID No. 6 or SEQ ID No. 16 or SEQ ID No. 17;
(d) a sequence of nucleotides that is a splice variant of (a), (b) or (c);
(e) a sequence of nucleotides that encodes a protease domain or a catalytically active portion thereof that includes a sequence of nucleotides having at least about 60%, 70%, 80%, 90% or 95% sequence identity with the sequence set forth in SEQ ID No. 5 or 15; and
(f) a sequence of nucleotides comprising degenerate codons of (a), (b), (c), (d) or (e).

74. The method of claim 68, wherein the polypeptide is selected from the group consisting of:
a polypeptide that consists essentially of a sequence of amino acids set forth in SEQ ID No. 6 or as amino acids 113-343 of SEQ ID No. 16 or as amino acids 376-642 of SEQ ID No. 16.
a polypeptide that consists essentially of a sequence of amino acids encoded by a sequence of nucleotides that hybridizes under conditions of high stringency to the sequence of nucleotides in SEQ ID No. 5 or set forth as nucleotides 360-1052 or 1148-1952 in SEQ ID No. 15;
a polypeptide that comprises the sequence of amino acids set forth as residues 376-642 SEQ ID No. 16;
a polypeptide that comprises a sequence of amino acids having at least about 60% sequence identity with the sequence of amino acids in SEQ ID No. 6 or the sequence of amino acids in SEQ ID No. 16; and
a polypeptide that is encoded by a sequence of nucleotides that is a splice variant of the sequence set forth in SEQ ID No. 15.

75. A method for identifying activators of a zymogen or inactive form of an MTSP20, comprising:
contacting a zymogen form or a proteolytically inactive form of an MTSP20 polypeptide of claim 1 or a polypeptide of SEQ ID No. 17 or a proteolytically active portion of the polypeptide with a substrate of the activated form of the polypeptide;
adding a test compound, wherein the test compound is added before, after or simultaneously with the addition of the substrate; and
detecting cleavage of the substrate, thereby identifying compounds that activate the zymogen.

76. The method of claim 75, wherein the substrate is a chromogenic substrate.

77. The method of claim 75, wherein the substrate is a L-pyroglutamyl-L-prolyl-L-arginine-p-nitroaniline hydrochloride.

78. The method of claim 75, wherein the test compound is a small molecule, a nucleic acid or a polypeptide.

79. A method for treating or preventing a neoplastic disease, in a mammal, comprising administering to a mammal an effective amount of an inhibitor of a polypeptide of claim 1 or a polypeptide of SEQ ID No. 17.

80. The method of claim 79, wherein the inhibitor is an antibody that specifically binds to the polypeptide, or a fragment or derivative of the antibody containing a binding domain thereof, wherein the antibody is a polyclonal antibody or a monoclonal antibody.

81. The method of claim 79, wherein the polypeptide is encoded by a sequence of nucleotides selected from the group consisting of:
(a) a sequence of nucleotides in SEQ ID No. 5 or SEQ ID No. 15 or a sequence of nucleotides that encodes the polyeptide of SEQ ID No. 17;
(b) a sequence of nucleotides that hybridizes under high stringency along its length or along at least about 70% of the full-length to the sequence of nucleotides in SEQ ID No. 5 or SEQ ID No. 15 or a sequence of nucleotides that encodes the polyeptide of SEQ ID No. 17;
(c) a sequence of nucleotides that encodes the polypeptide of SEQ ID No. 6 or SEQ ID No. 16 or SEQ ID No. 17;
(d) a sequence of nucleotides that is a splice variant of (a), (b) or (c);
(e) a sequence of nucleotides that encodes a protease domain or a catalytically active portion thereof that includes a sequence of nucleotides having at least about 60%, 70%, 80%, 90% or 95% sequence identity with the sequence set forth in SEQ ID No. 5 or 15; and
(f) a sequence of nucleotides comprising degenerate codons of (a), (b), (c), (d) or (e).

82. The method of claim 79, wherein the polypeptide is selected from the group consisting of:
a polypeptide that consists essentially of a sequence of amino acids set forth in SEQ ID No. 6 or as amino acids 113-343 of SEQ ID No. 16 or as amino acids 376-642 of SEQ ID No. 16.
a polypeptide that consists essentially of a sequence of amino acids encoded by a sequence of nucleotides that hybridizes under conditions of high stringency to the sequence of nucleotides in SEQ ID No. 5 or set forth as nucleotides 360-1052 or 1148-1952 in SEQ ID No. 15;
a polypeptide that comprises the sequence of amino acids set forth as residues 376-642 SEQ ID No. 16;
a polypeptide that comprises a sequence of amino acids having at least about 60% sequence identity with the sequence of amino acids in SEQ ID No. 6 or the sequence of amino acids in SEQ ID No. 16; and
a polypeptide that is encoded by a sequence of nucleotides that is a splice variant of the sequence set forth in SEQ ID No. 15.

83. A method of inhibiting tumor initiation, growth or progression or treating a malignant or pre-malignant condition, comprising administering an agent that inhibits activation cleavage of a protease domain of the zymogen form of an MTSP20 polypeptide of of claim 1 or a polypeptide of SEQ ID No. 17 or a proteolytically active portion of the polypeptide, upon cleavage, is proteolytically active, or inhibits an activity of the activated form of MTSP20 or a portion thereof that is proteolytically active.

84. The method of claim 83, wherein the condition is a condition of the breast, cervix, prostate, lung, ovary or colon.

85. The method of claim 83, wherein the agent is an antisense oligonucleotide, double-stranded RNA (dsRNA) or an antibody.

86. The method of claim 83, further comprising administering another treatment or agent selected from anti-tumor and anti-angiogenic treatments or agents.

87. The method of claims 83, wherein the polypeptide is encoded by a sequence of nucleotides selected from the group consisting of:
(a) a sequence of nucleotides in SEQ ID No. 5 or SEQ ID No. 15 or a sequence of nucleotides that encodes the polyeptide of SEQ ID No. 17;
(b) a sequence of nucleotides that hybridizes under high stringency along its length or along at least about 70% of the full-length to the sequence of nucleotides in SEQ ID No. 5 or SEQ ID No. 15 or a sequence of nucleotides that encodes the polyeptide of SEQ ID No. 17;
(c) a sequence of nucleotides that encodes the polypeptide of SEQ ID No. 6 or SEQ ID No. 16 or SEQ ID No. 17;
(d) a sequence of nucleotides that is a splice variant of (a), (b) or (c);
(e) a sequence of nucleotides that encodes a protease domain or a catalytically active portion thereof that includes a sequence of nucleotides having at least about 60%, 70%, 80%, 90% or 95% sequence identity with the sequence set forth in SEQ ID No. 5 or 15; and
(f) a sequence of nucleotides comprising degenerate codons of (a), (b), (c), (d) or (e).

88. The method of claim 83, wherein the polypeptide, wherein the polypeptide, wherein the polypeptide is selected from the group consisting of:
a polypeptide that consists essentially of a sequence of amino acids set forth in SEQ ID No. 6 or as amino acids 113-343 of SEQ ID No. 16 or as amino acids 376-642 of SEQ ID No. 16.
a polypeptide that consists essentially of a sequence of amino acids encoded by a sequence of nucleotides that hybridizes under conditions of high stringency to the sequence of nucleotides in SEQ ID No. 5 or set forth as nucleotides 360-1052 or 1148-1952 in SEQ ID No. 15;
a polypeptide that comprises the sequence of amino acids set forth as residues 376-642 SEQ ID No. 16;
a polypeptide that comprises a sequence of amino acids having at least about 60% sequence identity with the sequence of amino acids in SEQ ID No. 6 or the sequence of amino acids in SEQ ID No. 16; and
a polypeptide that is encoded by a sequence of nucleotides that is a splice variant of the sequence set forth in SEQ ID No. 15.

89. A method of identifying a compound that binds to the single-chain and/or a multi-chain form of an MTSP20 polypeptide and/or to a proteolytically active portion of a single-chain and/or multi-chain form of an MTSP20 polypeptide, comprising:
contacting a test compound with both a single-chain and multi-chain form of the MTSP20 polypeptide and/or proteolytically active portion thereof;
determining to which form(s) the compound binds; and
if it binds to a form of polypeptide, further determining whether the compound has at least one of the following properties:
(i) inhibits activation cleavage of a zymogen form of a protease domain of the polypeptide;
(ii) inhibits activity of a protease domain of a multi-chain or single-chain form; and
(iii) inhibits dimerization of the polypeptide, wherein the multi-chain form is a two, three or four chain polypepeptide.

90. A method of detecting neoplastic disease, comprising: detecting a polypeptide that comprises a polypeptide of claim 1 or the polyepeptide of SEQ ID No. 17 in or from a sample, wherein the amount, the form and/or activity detected differs from the amount, the form and/or activity of polypeptide detected from a subject who does not have neoplastic disease.

91. The method of claim 90, wherein the sample is selected from the group consisting of blood, urine, saliva, tears, synovial fluid, sweat, interstitial fluid, sperm, cerebrospinal fluid, ascites fluid, tumor tissue biopsy and circulating tumor cells.

92. The method of claim 90, wherein polypeptide consists essentially of a protease domain or a plurality thereof.

93. A method of diagnosing the presence of a pre-malignant lesion, a malignancy, or other pathologic condition in a subject, comprising:
obtaining a biological sample from the subject; and
exposing the sample or polypeptides isolated therefrom to a detectable agent that binds to a multi-chain and/or single-chain form of an MTSP20 polypeptide, wherein the pathological condition is characterized by the presence or absence of the multi-chain or single-chain form, wherein the multi-chain form is a two, three or four chain polypepeptide.

94. The method of claim 93, wherein the biological sample is selected from the group consisting of blood, urine, saliva, tears, synovial fluid, sweat, interstitial fluid, cerebrospinal fluid, a sperm sample, ascites fluid, tumor tissue biopsy and circulating tumor cells.

95. A method of monitoring tumor progression and/or therapeutic effectiveness, comprising detecting and/or quantifying the level, the form and/or activity of an MTSP20 polypeptide in a body tissue or fluid sample.

96. The method of claim 95, wherein the tumor is a tumor of the breast, cervix, prostate, lung, ovary or colon.

97. The method of claim 95, wherein the body fluid is blood, urine, sweat, saliva, cerebrospinal fluid and synovial fluid.

98. A transgenic non-human animal, comprising heterologous nucleic acid encoding a polypeptide of claim 1 or the polypeptide of SEQ ID No. 17.

99. A probe or primer that:
comprises at least 14 contiguous nucleotides or modified nucleotides that are identical to a contiguous sequence of nucleotides encoding the amino acids 624-642 of SEQ ID No. 16;
comprises at least 16 contiguous nucleotides or modified nucleotides that are identical to a contiguous sequence of nucleotides encoding the amino acids 624-642 of SEQ ID No. 16; or comprises at least 30 contiguous nucleotides or modified nucleotides that are identical to a contiguous sequence of nucleotides encoding the amino acids 624-642 of SEQ ID No. 16.

100. A method for treating or preventing a disease or disorder associated with undesired and/or uncontrolled angiogenesis or neovascularization, in a mammal, comprising administering to a mammal an effective amount of an inhibitor of an MTSP20 polypeptide.

101. The method of claim 100, wherein the inhibitor is selected from the group consisting of an antibody or a fragment or derivative thereof that specifically binds to the MTSP20, an antisense nucleic acid, a double-stranded RNA molecule.

102. The method of claim 100, wherein the undesired angiogenesis is associated with disorders selected from the group consisting of solid neoplasms, vascular malformations and cardiovascular disorders, chronic inflammatory diseases and aberrant wound repairs, circulatory disorders, crest syndromes, dermatological disorders and ocular disorders.

103. The method of claim 102, wherein:
the vascular malformations and cardiovascular disorders are selected from the group consisting of angiofibroma, angiolipoma, atherosclerosis, restenosis/reperfusion injury, arteriovenous malformations, hemangiomatosis and vascular adhesions, dyschondroplasia with vascular hamartomas (Fafucci's syndrome), hereditary hemorrhagic telangiectasia (Rendu-Osler-Weber syndrome) and Von Hipple Lindau syndrome;
the chronic inflammatory diseases are selected from the group consisting of diabetes mellitus, hemophiliac joints, inflammatory bowel disease, nonhealing fractures, periodontitis (rapidly progressing and juvenile), psoriasis, rheumatoid arthritis, venous stasis ulcers, granulations-burns, hypertrophic scars, liver cirrhosis, osteoradionecrosis, postoperative adhesions, pyogenic granuloma and systemic sclerosis;
the circulatory disorder is Raynaud's phenomenon;
the crest syndromes are selected from the group consisting of calcinosis, esophageal, dyomotiloty, sclerodactyly and teangiectasis;
the dermatological disorders are selected from the group consisting of systemic vasculitis, scleroderma, pyoderma gangrenosum, vasculopathy, venous, arterial ulcers, Sturge-Weber syndrome, Port-wine stains, blue rubber bleb nevus syndrome, Klippel-Trenaunay-Weber syndrome and Osler-Weber-Rendu syndrome;
the ocular disorders are selected from the group consisting of blindness caused by ocular neovascular disease, corneal graft neovascularization, macular degeneration in the eye, neovascular glaucoma, trachoma, diabetic retinopathy, myopic degeration, retinopathy of prematurity, retrolental fibroplasia and corneal neovascularization.

104. A combination, comprising:
a) an inhibitor of the proteolytic activity of the MTSP20; and
b) an another treatment or agent selected from anti-tumor and anti-angiogenic treatments or agents.

105. The combination of claim 104, wherein the inhibitor and the anti-angiogenic agent are formulated in a single pharmaceutical composition or each is formulated. in separate pharmaceutical compositions.

106. The combination of 104, wherein the inhibitor is selected from antibodies, antisense oligonucleotides and double-stranded RNA molecules.

107. A computational method for screening, comprising:
assessing the interaction of a test compound with a computer-simulated polypeptide that has the sequence of amino acids of a polypeptide of claim 1 or an MTSP20 polypeptide that comprises SEQ ID No. 17; and
identifying test compounds that interact with the polypeptide, wherein assessment is effected in silico.

108. The polypeptide of claim 1, wherein:
the MTSP20 portion of the polypeptide consists essentially of protease domain 2 (PD2) of the MTSP20 or a catalytically active portion thereof.
